(19) United States
(12) Patent Application Publication
(10) Pub. No.: US 2025/0325707 A1
(43) Pub. Date: Oct. 23, 2025

(54) Solving Brain Circuit Function and Dysfunction With Computational Modeling and Optogenetic Functional Magnetic Resonance Imaging

(71) Applicant: The Board of Trustees of the Leland Stanford Junior University, Stanford, CA (US)

(72) Inventors: Jin Hyung Lee, Palo Alto, CA (US); Qin Liu, Redwood City, CA (US)

(21) Appl. No.: 19/112,496

(22) PCT Filed: Sep. 28, 2023
(86) PCT No.: PCT/US2023/075410

§ 371 Date: Mar. 17, 2025

Related U.S. Application Data
(60) Provisional application No. 63/377,960, filed on Sep. 30, 2022.

(51) Int. Cl. A61K 48/00; A61B 5/055; A61N 1/05; A61N 1/36; A61N 5/06; A61N 5/067; C12N 15/86; G01R 33/48; G01R 33/56; G16H 30/20

(52) U.S. Cl. CPC A61K 48/0058; A61B 5/055; A61N 1/0534; A61N 1/36067; A61N 5/0622; A61N 5/067; C12N 15/86; G01R 33/4806; G01R 33/5608; G16H 30/20; A61N 2005/063; A61N 2005/0647; C12N 2750/14143; C12N 2820/007

(57) ABSTRACT

Methods, systems, and devices, including computer programs encoded on a computer storage medium are provided for optimizing neurostimulation therapy for treatment of neurological and neurodegenerative diseases. Joint dynamic causal modeling and biophysics modeling are used for optimization of the stimulation targets and parameters. In particular, methods of performing neuromodulation to suppress b-band oscillations in the brain of a subject are provided.

### CROSS REFERENCE TO RELATED APPLICATION

This application claims benefit of U.S. Provisional Patent Application No. 63/377,960, filed Sep. 30, 2022, which application is incorporated herein by reference in its entirety.

### STATEMENT REGARDING FEDERALLY SPONSORED RESEARCH OR DEVELOPMENT

This invention was made with Government support under contracts MH114227, NS116783, NS091461, EB030884, and AG064051 awarded by the National Institutes of Health. The Government has certain rights in the invention.

### BACKGROUND OF THE INVENTION

Neuronal firing rates and patterns are both crucial in untangling the dynamics of brain functions and dysfunctions (Engel et al., Nat Rev Neurosci 2, 704-716 (2001); Cannon et al., Eur J Neurosci 39, 705-719 (2014); Schoffelen et al., Science 308, 111-113 (2005)). For example, in Parkinson's disease (PD), altered firing rates and patterns, in the form of abnormal β-band (12-30 Hz) oscillations, have been observed across the cortico-basal-ganglia-thalamus (CBT) network (Albin et al., Trends Neurosci 12, 366-375 (1989); DeLong, Trends Neurosci 13, 281-285 (1990); Kühn et al., Brain 127, 735-746 (2004)). Understanding how the CBT network mediate neural oscillations and how the neural oscillations are related to the firing rates of individual cell populations can facilitate decoding of the circuit mechanism underlying PD pathology and improve neuromodulation treatments.

### SUMMARY OF THE INVENTION

Methods, systems, and devices, including computer programs encoded on a computer storage medium are provided for optimizing neurostimulation therapy for treatment of neurological and neurodegenerative diseases. Joint dynamic causal modeling and biophysics modeling are used for optimization of the stimulation targets and parameters. In particular, methods of performing neuromodulation to suppress β-band oscillations in the brain of a subject are provided.

In one aspect, a method of suppressing β-band oscillations in the brain of a subject are provided, wherein optogenetic neuromodulation of medium spiny neurons is performed according to a method comprising: (a) optogenetically inhibiting D1-medium spiny neurons (D1-MSNs) in a globus pallidus internal (GPi) region of the brain of the subject, wherein D1-MSN mediated β-band oscillations are suppressed; (b) optogenetically inhibiting D2-medium spiny neurons (D2-MSNs) in a globus pallidus external (Gpe) region of the brain of the subject, wherein D2-MSN mediated β-band oscillations are suppressed; (c) optogenetically stimulating the D1-MSNs periodically in the Gpe region of the brain of the subject, wherein D1-MSN mediated β-band oscillations are suppressed; or (d) optogenetically stimulating the D1-MSNs and the D2-MSNs randomly in the Gpe region of the brain of the subject, wherein D1-MSN mediated β-band oscillations and D2-MSN mediated β-band oscillations are suppressed; or any combination of (a)-(d).

In certain embodiments, the method comprises: (a) optogenetically inhibiting the D1-MSNs in the GPi region of the brain of the subject, wherein the D1-MSN mediated β-band oscillations are suppressed; (b) optogenetically inhibiting the D2-MSNs in the Gpe region of the brain of the subject, wherein the D2-MSN mediated β-band oscillations are suppressed; (c) optogenetically stimulating the D1-MSNs periodically in the Gpe region of the brain of the subject, wherein the D1-MSN mediated β-band oscillations are suppressed; and (d) optogenetically stimulating the D1-MSNs and the D2-MSNs randomly in the Gpe region of the brain of the subject, wherein the D1-MSN mediated β-band oscillations and the D2-MSN mediated β-band oscillations are suppressed.

In certain embodiments, optogenetically inhibiting the D1-MSNs or the D2-MSNs in the Gpi region or the Gpe region comprises: introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and illuminating the light-responsive ion channel with light at a wavelength that activates the light-responsive ion channel, wherein conduction of ions by the light-responsive ion channel in response to absorption of light results in hyperpolarization and inhibition of the D1-MSNs or the D2-MSNs.

In certain embodiments, the light-responsive ion channel is a light-responsive anion-conducting opsin or a light-responsive proton conductance regulator.

In certain embodiments, the light-responsive anion-conducting opsin conducts chloride ions (Cl⁻).

In certain embodiments, the anion-conducting opsin is an anion-conducting channelrhodopsin or halorhodopsin.

In certain embodiments, the halorhodopsin is a Natronomonas pharaonis halorhodopsin (NpHR), enhanced NpHR (eNpHR) 1.0, eNpHR 2.0, or eNpHR 3.0.

In certain embodiments, the anion-conducting channelrhodopsin is iC1C2, SwiChR, SwiChR++, or iC++.

In certain embodiments, the light-responsive proton conductance regulator is a bacteriorhodopsin or an archaerhodopsin.

In certain embodiments, the light-responsive proton conductance regulator is Arch from Halorubrum sodomense, ArchT from Halorubrum sp., TP009 from Leptosphaeria maculans, or Mac from Leptosphaeria maculans.

In certain embodiments, optogenetically stimulating the D1-MSNs or the D2-MSNs in the Gpi region or the Gpe region comprises: introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and illuminating the light-responsive ion channel with light at a wavelength that activates the light-responsive ion channel, wherein conduction of ions by the light-responsive ion channel in response to absorption of light results in depolarization and activation of the D1-MSNs or the D2-MSNs.

In certain embodiments, the light-responsive ion channel is a light-responsive cation-conducting opsin.

In certain embodiments, the light-responsive cation-conducting opsin conducts calcium cations (Ca²⁺).

In certain embodiments, the light-responsive cation-conducting opsin is a light-responsive cation-conducting channelrhodopsin.

In certain embodiments, the light-responsive cation-conducting channelrhodopsin is a Chlamydomonas reinhardtii channelrhodopsin or a Volvox carteri channelrhodopsin.

In certain embodiments, the light-responsive cation-conducting channelrhodopsin is a Chlamydomonas reinhardtii channelrhodopsin-1 (ChR1), a Chlamydomonas reinhardtii channelrhodopsin-2 (ChR2), a Volvox carteri channelrhodopsin-1 (VChR1), or a chimeric ChR1-VChR1 channelrhodopsin.

In certain embodiments, the polynucleotide encoding the light-responsive ion channel is provided by a viral vector.

In certain embodiments, the viral vector is a lentiviral vector or an adeno-associated viral (AAV) vector.

In certain embodiments, the viral vector is stereotactically injected into the retrosplenial cortex.

In certain embodiments, the vector further comprises a neuron-specific promoter operably linked to the polynucleotide encoding the light-responsive ion channel.

In certain embodiments, expression of the light-responsive ion channel is inducible.

In certain embodiments, illuminating the light-responsive ion channel comprises delivering light from a light source to the light-responsive ion channel using a fiber-optic-based optical neural interface.

In certain embodiments, the light source is a solid-state diode laser.

In certain embodiments, the subject has Parkinson's disease.

In certain embodiments, optogenetically inhibiting the D1-MSNs comprises sustained shunting inhibition of the D1-MSNs in the GPi region of the brain of the subject.

In certain embodiments, optogenetically inhibiting the D2-MSNs comprises sustained shunting inhibition of the D2-MSNs in the GPe region of the brain of the subject.

In certain embodiments, optogenetically stimulating the D1-MSNs periodically in the Gpe region of the brain of the subject comprises performing periodic stimulation at a frequency of 130 Hz.

In certain embodiments, optogenetically stimulating the D1-MSNs and the D2-MSNs randomly in the Gpe region of the brain of the subject comprises using a plurality of stimulation sequences with randomly spaced pulses of 130 Hz, wherein each neuron is stimulated with one of the stimulation sequences such that synchronized neurons are decoupled.

In certain embodiments, optogenetically stimulating the D1-MSNs or the D2-MSNs comprises direct activation of the D1-MSNs or the D2-MSNs with a strength of 500 pA, 700 pA, or 900 pA.

In another aspect, a method of treating Parkinson's disease in a subject is provided, the method comprising: positioning a first electrode at a first location in a globus pallidus internal (GPi) region of the brain of the subject to deliver electrical stimulation to D1-medium spiny neurons in the Gpi region; positioning a second electrode at a second location in a globus pallidus external (Gpe) region of the brain of the subject to deliver electrical stimulation to D1-medium spiny neurons and D2-medium spiny neurons in the Gpe region; and applying electrical stimulation to the Gpi region of the brain of the subject using the first electrode and applying electrical stimulation to the Gpe region of the brain of the subject using the second electrode in a manner effective to suppress β-band oscillations to treat Parkinson's disease.

In certain embodiments, the electrical stimulation is applied with the first electrode or the second electrode unilaterally or bilaterally.

In certain embodiments, the first electrode or the second electrode is a depth electrode or a surface electrode.

In certain embodiments, the first electrode or the second electrode is a non-brain penetrating surface electrode array or a brain-penetrating electrode array.

In certain embodiments, the first electrode is placed on a surface of the Gpi region.

In certain embodiments, the second electrode is placed on a surface of the Gpe region.

In certain embodiments, the method further comprises assessing effectiveness of the treatment in the subject using a visual analog scale, a verbal rating scale, a Movement Disorder Society-Sponsored Revision of the Unified Parkinson's Disease Rating Scale (MDS-UPDRS), a Hoehn and Yahr (HnY) scale, or a Montreal Cognitive Assessment (MoCA) scale.

In another aspect, a computer implemented method for modeling propagation of β-band oscillations in the brain of a subject and response to neuromodulation is provided, the computer performing steps comprising: a) receiving functional magnetic resonance imaging data of neural activity before optogenetic stimulation and during optogenetic stimulation of D1-medium spiny neurons (D1-MSNs) and D2-medium spiny neurons (D2-MSNs) in a caudate putamen (CPu) region, an external globus pallidus (GPe) region, an internal globus pallidus (GPi) region, a subthalamic nucleus (STN) region, a substantia nigra pars reticulata (SNr) region, a thalamus (THL) region, and a motor cortex (MCX) region of the brain of the subject; b) performing spectral dynamic causal modeling of effective connectivity strengths among the CPu region, the GPe region, the GPi region, the STN region, the SNr region, the THL region, and the MCX region; c) receiving experimental electrophysiological data for the CPu region; d) estimating effective connectivity strengths of GABAergic connections using the experimental electrophysiological data for the CPu region; e) estimating effective connectivity strengths of glutamatergic connections using dynamic causal modeling of the functional magnetic resonance imaging data; f) performing biophysics modeling using a Hodgkin-Huxley model to generate simulated electrophysiology data using the effective connectivity strength estimates; g) optimizing GABAergic projections iteratively until the simulated electrophysiology data matches the experimental electrophysiological data for the CPu region; h) calculating a temporal profile of average power of beta-band frequencies for each neuron in the CPu region, the GPe region, the GPi region, the STN region, the SNr region, the THL region, and the MCX region; and i) comparing total amount of co-occurred beta-band oscillation power before optogenetic stimulation to co-occurred beta-band oscillation power during optogenetic stimulation to model the propagation of β-band oscillations in the brain of the subject.

In certain embodiments, the D1-MSN are Huxley-Hudgkin neurons.

In certain embodiments, the experimental electrophysiology data comprise single-neuron recordings.

In certain embodiments, the single-neuron recordings are from GABAergic neurons of the CPu region.

In certain embodiments, the glutamatergic connections are modeled as 1-to-1 connections with connection strengths proportional to effective connectivity estimated by the DCM.

In certain embodiments, the CPu-GPi/GPe and GPi/SNr-thalamus GABAergic projections are modeled as 1-to-n diffusive projections with connectivity strength wGABA, where n and wGABA are free parameters, wherein N and wGABA are searched across parameter space until the simulated spike rates match statistically with the single-neuron recordings.

In certain embodiments, the optogenetic stimulation comprises optogenetically inhibiting the D1-MSNs in the GPi region of the brain of the subject.

In certain embodiments, optogenetically inhibiting the D1-MSNs comprises sustained shunting inhibition of the D1-MSNs in the GPi region of the brain of the subject.

In certain embodiments, the optogenetic stimulation comprises optogenetically inhibiting the D2-MSNs in the GPe region of the brain of the subject.

In certain embodiments, optogenetically inhibiting the D2-MSNs comprises sustained shunting inhibition of the D2-MSNs in the GPe region of the brain of the subject.

In certain embodiments, the optogenetic stimulation comprises direct activation of the D1-MSNs or the D2-MSNs with a strength of 500 pA, 700 pA, or 900 pA.

In certain embodiments, the optogenetic stimulation comprises optogenetically stimulating the D1-MSNs periodically in the Gpe region of the brain of the subject.

In certain embodiments, the optogenetic stimulation comprises optogenetically stimulating the D1-MSNs and the D2-MSNs randomly in the Gpe region of the brain of the subject using a plurality of stimulation sequences with randomly spaced pulses, wherein each neuron is stimulated with one of the stimulation sequences such that synchronized neurons are decoupled.

In certain embodiments, the optogenetic stimulation is performed with periodic stimulation at a frequency of 130 Hz.

In another aspect, a system for modeling propagation of β-band oscillations in the brain of a subject using a computer implemented method, described herein, is provided, the system comprising: a) a storage component for storing data, wherein the storage component has instructions for modeling propagation of β-band oscillations based on analysis of the functional magnetic resonance imaging data and the experimental electrophysiology data stored therein; b) a computer processor for processing the functional magnetic resonance imaging data and the experimental electrophysiology data using one or more algorithms, wherein the computer processor is coupled to the storage component and configured to execute the instructions stored in the storage component in order to receive the inputted functional magnetic resonance imaging data and the experimental electrophysiology data and analyze the data according to a computer implemented method described herein; and c a display component for displaying the information regarding the propagation of β-band oscillations in the brain of the subject.

In another aspect, a non-transitory computer-readable medium comprising program instructions that, when executed by a processor in a computer, causes the processor to perform the method, described herein, is provided.

In another aspect, a kit comprising a non-transitory computer-readable medium and instructions for modeling the propagation of β-band oscillations from functional magnetic resonance imaging data and experimental electrophysiology data, as described herein, is provided.

### BRIEF DESCRIPTION OF THE DRAWINGS

FIGS. 1A-1E. In vivo electrophysiology data suggest that the direct pathway can mediate β-band oscillations. (FIG. 1A) Illustrations of the in vivo extracellular recordings locations in CPu, GPi, thalamus, and STN, respectively. (FIG. 1B) Histograms of representative neurons in CPu, GPi, thalamus, and STN, respectively. The neuronal activity of all four ROIs increase during D1-MSN stimulations. To the right of each histogram are the corresponding firing rates before, during, and after stimulations for the selected neurons (***p<0.001, one-tailed paired t test, mean±SEM). (FIG. 1C) SPIKE-synchronization profiles of ROIs during D1- and D2-MSN stimulations, respectively. D1-MSN stimulations induces higher-than-baseline synchronization in all 4 ROIs. To the right of each temporal profile are the corresponding averaged SPIKE-synchronization before, during, and after stimulations (***p<0.001, one-tailed paired t test, mean±SEM). (FIG. 1D) Power spectral profiles of CPu, GPi, thalamus, and STN during D1-MSN stimulations, respectively. The 20 Hz optogenetic stimulation in D1-MSN drives oscillations of the same frequency in GPi and thalamus, but not in STN. (FIG. 1E) When aligned with laser pulse cycles, the spike trains of CPu, GPi, and thalamus exhibit phase-locking activity during D1-MSN stimulations. Spikes of CPu align with laser onset in each phase cycle. Spikes of GPi and thalamus exhibit ˜30 and ˜60 ms phase delay relative to the laser onset, respectively.

FIGS. 2A-2F. In vivo electrophysiology data suggest that the indirect pathway can partially mediate β-band oscillations. (FIG. 2A) Illustrations of the in vivo extracellular recordings locations in CPu, GPi, thalamus, and STN, respectively. (FIG. 2B) Histograms of representative neurons of CPu, GPi, thalamus, and STN, respectively. The neuronal activity of GPi, thalamus and STN decrease during D2-MSN stimulations. To the right of each histogram are the corresponding firing rates before, during, and after stimulations for the selected neurons (***p<0.001, one-tailed paired t test, mean±SEM). (FIG. 2C) SPIKE-synchronization profiles of ROIs during D2-MSN stimulation. D2-MSN stimulation induces higher synchronization in CPu, but lower-than-baseline synchronization levels in GPi and STN. To the right of each temporal profile are the corresponding averaged SPIKE-synchronization before, during, and after stimulations (***p<0.001, one-tailed paired t test, mean±SEM). (FIG. 2D) Power spectral density of ROIs during D2-MSN. 20 Hz peaks are observed in CPu and STN, but the synchronization does not propagate to thalamus. (FIG. 2E) CPu and STN exhibit in-phase activity during D2-MSN stimulations. Spikes of CPu align with laser onset in each phase cycle. Spikes of GPi and thalamus have no preferred phase during D2-MSN stimulations. (FIG. 2F) STN neurons exhibit hypoactivity and 20 Hz synchronization simultaneously during D2-MSN stimulations. The histogram and power spectral profile of one representative STN neuron are shown in the right two panels.

FIGS. 3A-3C. Spiking-level biophysics model built and optimized based on ofMRI and single-neuron recording data. (FIG. 3A) Graph and matrix representations of the structural network of the biophysics model. 20 Hz optogenetic stimulation targeted D1- or D2-MSN in CPu. Glutermatergic connections are modeled as 1-to-1 projections with strengths optimized by the DCM model based on ofMRI time series. GABAergic connections are modeled as 1-to-n projections with parameters optimized utilizing single-neuron recordings. (FIG. 3B) The left panel shows the average PSD profiles of 118 neurons identified from our recordings over a single trial during baseline and stimulation periods. We used the PSD profiles from these recordings to simulate a realistic input for our model. The middle panel shows the resulting average PSD of 100 simulated CPu neurons. In each trial, we simulate 20 seconds baseline, 20 seconds D1-/D2-MSN stimulation period, and another 20 seconds without stimulation. (FIG. 3C) Flowchart showing experimental data and its relationship to the modeling process. We used optogenetic fMRI data and DCM to estimate glutamatergic connection strengths. Parameters of GABAergic connections, fanout ratio and strength, are optimized by matching synthetic spike trains with single-neuron recordings. Through this approach, we build a single-cell spiking model whose parameters are optimized with ofMRI and electrophysiology data and the model converges when model spike trains match experimental data spike rates.

FIGS. 4A-41. Model successfully replicates D1- and D2-MSN stimulation experimental results across the whole CBT network. (FIG. 4A) Schematics of optogenetic stimulations and simulated single-neuron recording locations in CPu, GPi, thalamus, and STN, respectively. (FIGS. 4B and 4E) Histograms of simulated neuronal activity in CPu, GPi, thalamus, and STN during D1- and D2-MSN stimulations, respectively. Neuronal activity increases are observed in all four ROIs during D1-MSN stimulations (FIG. 4B). Neuronal activity decreases are observed in GPi, thalamus, and STN during D2-MSN stimulations (FIG. 4E). To the right of each histogram are the corresponding firing rates before, during, and after stimulations (***p<0.001, one-tailed paired t test, mean±SEM). (FIGS. 4C and 4F) Power spectral profiles of CPu, GPi, thalamus, and STN during simulated D1- and D2-MSN stimulations, respectively. 20 Hz optogenetic D1-MSN stimulation drives oscillations of the same frequency in CPu and synchronizes downstream GPi and thalamus (FIG. 4C). 20 Hz synchronization is observed in CPu and STN, but not in thalamus (FIG. 4F) during D2-MSN stimulations. (FIGS. 4D and 4G) Comparisons between the spike rates of experiment and simulation of D1-MSN (FIG. 4D) and D2-MSN (FIG. 4G) stimulations show that the simulated spike rates match the experimental recordings. (FIG. 4H) Simulation results suggest β-band oscillations being mediated by phase-locked rebound firings along both the direct and indirect pathways. (FIG. 4I) The parameter space of connectivity strength and axonal density in target region predicts that GPe-STN connection may have higher axonal branching than other β-oscillation mediating connections.

FIGS. 5A-5E. Optogenetically enhanced spontaneous activities validate the propagation pathways of β-band oscillations. (FIG. 5A) Activation of SSFO D1-MSN induced broad-band activity increase in CPu, SNr, thalamus, and GPi. (FIG. 5B) Activation of SSFO D2-MSN induced broad-band activity decrease in CPu and thalamus. (FIG. 5C) We identified co-occurring epochs of high beta-power from simultaneous extracellular recordings of two region pairs. An example pair is shown for a selected time window from CPu-GPi simultaneous recordings during D1-MSN activation. Cooccurring beta-power peaks higher than the threshold are marked with a grey arrow. (FIG. 5D) D1-MSN activation significantly increased the number of co-occurring beta epochs/10 s between CPu-GPi, CPu-SNr, and CPu-thalamus pairs (***p<0.001, two-tailed paired t test). (FIG. 5E) D2-MSN activation significantly decreased the number of co-occurring beta epochs/10 s between CPu-SNr and CPu-thalamus pairs (***p<0.001, two-tailed paired t test).

FIGS. 6A-6F. Simulated neuromodulation experiments successfully predict optimal neuromodulation targets for the interruption of β-band oscillations. (FIG. 6A) Schematics of experimental design to interrupt the direct pathway>-band oscillations using opto-inhibition (left) and opto-stimulation (right). STN, motor cortex, GPi, and GPe were selected as candidate targets for neuromodulations. (FIG. 6B) Simulated opto-inhibitions show that GPi is the best site for opto-inhibition to interrupt the β-band oscillations along the direct pathway. (FIG. 6C) Simulated opto-stimulations show that both periodic and random stimulation of GPe can suppress the β-band oscillations along the direct pathway. (FIG. 6D) Schematics of experimental design to interrupt the indirect pathway mediated β-band oscillation using opto-inhibition (left) and opto-stimulation (right). STN, motor cortex, GPi, and GPe were selected a s candidate targets for neuromodulations. (FIG. 6E) Simulated opto-inhibitions show that GPe is the best site for opto-inhibition to interrupt the β-band oscillations along the indirect pathway. (FIG. 6F) Simulated opto-stimulations show that only random stimulation of GPe can suppress the β-band oscillations along the indirect pathway. The error bands represent 95% confidence intervals.

FIGS. 7A-7F. Optogenetic functional magnetic resonance imaging bridges scale. (FIG. 7A) Optogenetics enable cell-type-specific stimulations, such as the selective targeting of D1- or D2-MSNs in the striatum. (FIG. 7B) With selective stimulations of D1- or D2-MSNs in the striatum, mouses show contraversive or ipsiversive rotations, respectively (D1-MSN stim: n=12 animals, mean±SEM, ***p<0.001, two-tailed paired t test; D2-MSN stim: n=11 animals, mean±SEM, *p<0.05, **p<0.005, two-tailed paired t test). (FIG. 7C) Optogenetic fMRI technology combines optogenetic stimulations with fMRI readout. (FIG. 7D) ofMRI with selective stimulation of D1- and D2-MSN results in distinct brain-wide activities that are associated with distinct behavior (n=12 animals). Group-wise phase maps, which were thresholded to only active voxels within the brain, depict the heterogeneity in the temporal dynamics of the evoked responses. (FIG. 7E) Time series of any region can be extracted from the four-dimensional fMRI data. (FIG. 7F) Electrophysiology recordings mirror fMRI response in polarity of neural activity change. This figure is based on Lee et al. (2).

FIGS. 8A-8G. Computational modeling of ofMRI data reveals brain-wide functional interaction dynamics. (FIG. 8A) Cortico-basal-ganglia-thalamus network involves a large number of network nodes across the brain. (FIG. 8B) Direct and indirect pathways' anatomical connectivity involve large number of common anatomical regions with distinct cell types in caudate putamen (CPu). (FIG. 8C) Anatomical connections were used as a priori generative network model. In addition to direct and indirect pathways shown in (B), other established anatomical connections, e.g., hyper-direct pathway, were also included. (FIG. 8D) DCM generated fMRI time series closely match experimental ofMRI time series. (FIG. 8E) DCM utilized ofMRI data to estimate the causal influence (effective connectivity) among regions of interest during D1- and D2-MSN stimulations, respectively. (FIGS. 8F and 8G) The graph and matrix representations of effective connectivity network for (F) D1-MSN, and (G) D2-MSN stimulations, respectively. Significant and close-to-significant represent parameters with p<0.05 and 0.05≤p<0.10, respectively (one-sample t test, multiple comparison correction across connections with FDR p<0.10). CPu: caudate putamen; GPe: external globus pallidus; GPi: internal globus pallidus; STN: subthalamic nucleus; SNr: substantia nigra; THL: thalamus; CTX: cortex. This figure is based on Bernal-casas et al. (4).

FIGS. 9A-9D. Brain circuit function modeling at the single-cell-spiking level can be made possible through a multi-scale approach. (FIG. 9A) Locations of optogenetic stimulation and in vivo extracellular recordings for the cortico-basal-ganglia-thalamus network study are schematically illustrated. (FIG. 9B) Single-cell-spiking level modeling with ofMRI and single-unit recordings data is exemplified. A large-scale model built with ofMRI data is expanded to single-neuron level biophysical model. Each ROI consists of many simulated single neurons. The model is validated by directly comparing simulated spiking trains with experimental data. (FIG. 9C) Models can allow experimental single-unit recording data and simulated data to be directly compared. (FIG. 9D) Models should be designed so that the spike rates of all ROIs simulated by the single-cell spiking level biophysical model statistically match experimental data. ofMRI combined with biophysics modeling can enable successful reproduction of the single-cell-spiking level dynamics induced by cell type specific optogenetic stimulations such as D1- and D2-MSN stimulations.

FIGS. 10A-10F. To understand pathology function interaction, whole-brain pathology dynamics can be modeled alongside ofMRI. (FIG. 10A) α-synuclein PFFs injected into seed locations induce pathology at various time points post-injection, which can then be captured by iDISCO tissue clearing and light-sheet fluorescent microscopy (LSFM). Machine-learning based, automatic segmentation and registration techniques can streamline the quantification of each pathological marker within the Allen Reference Atlas (ARA). (FIG. 10B) Comparisons of averaged heatmaps across cohorts can depict whole-brain pathology changes over many months after the injection. (FIG. 10C) Modeling of longitudinal data based on whole-brain anatomical connectivity can capture regional differences in pathology. (FIG. 10D) Reweighting the connectivity matrix can allow for encoding of genetic contribution within a model. (FIG. 10E) Whole-brain colocalization analysis between optogenetic-stimulation induced alpha-synuclein pathology change and optogenetic fMRI activity can reveal pathology function relationship. In this example, positive activity is colocalized with decreases in pathology, while negative activity is highly colocalized with increases in pathology. (FIG. 10F) Montages of the modulated alpha-synuclein pathology and ofMRI brain activity maps show high degree of colocalization with opposite polarity.

FIG. 11. Schematic of the technological approach. Optogenetic fMRI and single neuron recordings are used with dynamic causal modeling and biophysics modeling to optimize neuromodulation parameters.

FIGS. 12A-12F. Simulated neuromodulation experiments successfully predict optimal neuromodulation targets for the interruption of β-band oscillations. (FIG. 12A) Schematics of experimental design to interrupt the direct pathway β-band oscillations using opto-inhibition (left) and opto-stimulation (right). STN, motor cortex, GPi, and GPe were selected as candidate targets for neuromodulations. (FIG. 12B) Simulated opto-inhibitions show that GPi is the best site for opto-inhibition to interrupt the β-band oscillations along the direct pathway. (FIG. 12C) Simulated opto-stimulations show that both periodic and random stimulation of GPe can suppress the β-band oscillations along the direct pathway. (FIG. 12D) Schematics of experimental design to interrupt the indirect pathway mediated β-band oscillation using opto-inhibition (left) and opto-stimulation (right). STN, motor cortex, GPi, and GPe were selected a s candidate targets for 5 neuromodulations. (FIG. 12E) Simulated opto-inhibitions show that GPe is the best site for opto-inhibition to interrupt the β-band oscillations along the indirect pathway. (FIG. 12F) Simulated opto-stimulations show that only random stimulation of GPe can suppress the β-band oscillations along the indirect pathway. The error bands represent 95% confidence intervals.

### DETAILED DESCRIPTION OF THE INVENTION

Methods, systems, and devices, including computer programs encoded on a computer storage medium are provided for optimizing neurostimulation therapy for treatment of neurological and neurodegenerative diseases. Joint dynamic causal modeling and biophysics modeling are used for optimization of the stimulation targets and parameters. In particular, methods of performing neuromodulation to suppress-band oscillations in the brain of a subject are provided.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a neuron” includes a plurality of such neurons and reference to “the measurement” includes reference to one or more measurements and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about,” particularly in reference to a given quantity, is meant to encompass deviations of plus or minus five percent.

The term “connectivity” refers to causal, functional, or directed coupling between brain regions and/or neuronal populations. Neural processing may involve an integrated network of several regions of the brain and functional connectivity of spatially remote brain regions. Analysis of functional connectivity may involve detecting inter-regional and intra-regional neural interactions involved in brain activity during particular cognitive or motor tasks or from spontaneous activity during rest. Neural inter-regional and intra-regional “connectivity strengths” may be estimated from dynamic causal modeling of brain function.

“Neural activity” as used herein, may refer to electrical activity of a neuron (e.g., changes in membrane potential of the neuron), as well as indirect measures of the electrical activity of one or more neurons. Thus, neural activity may refer to changes in field potential, changes in intracellular ion concentration (e.g., intracellular calcium concentration), and/or changes in magnetic resonance induced by electrical activity of neurons, as measured by, e.g., blood oxygenation level dependent (BOLD) signals in functional magnetic resonance imaging.

The terms “individual”, “subject”, “host”, and “patient”, are used interchangeably herein and refer to any subject with a brain, including invertebrates and vertebrates such as, but not limited to, arthropods (e.g., insects, crustaceans, arachnids), cephalopods (e.g., octopuses, squids), amphibians (e.g., frogs, salamanders, caecilians), fish, reptiles (e.g., turtles, crocodilians, snakes, amphisbaenians, lizards, tuatara), mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. In some cases, the methods of the invention find use in experimental animals, in veterinary application, and in the development of animal models for disease, including, but not limited to, rodents including mice, rats, and hamsters; primates, and transgenic animals.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with Parkinson's disease) as well as those in which prevention is desired (e.g., those with a genetic predisposition to developing Parkinson's disease).

By “therapeutically effective dose or amount” of electrical stimulation (e.g., delivered optogenetically or with an electrode) is intended an amount that, when the electrical stimulation is administered, as described herein, brings about a positive therapeutic response in the treatment of Parkinson's disease such as an amount that suppresses β-band oscillations. Additionally, a positive therapeutic response in the treatment of Parkinson's disease may include a reduction in symptoms of Parkinson's disease such as reduced tremor, rigidity, slowness of movement, and/or difficulty with walking, improved sleep, and/or increased survival of neurons. The exact amount required will vary from subject to subject, depending on the species, age, and general condition of the subject, the severity of the condition being treated, the particular mode of administration, and the like. An appropriate “effective” amount in any individual case may be determined by one of ordinary skill in the art using routine experimentation, based upon the information provided herein.

Modeling Brain Function Using Dynamic Causal Modeling in Combination with Biophysics Modeling

Methods are provided for using dynamic causal modeling in combination with biophysics modeling to generate improved models of brain function and response to neuromodulation therapy. In particular, the subject methods can be used to integrate brain function measurements by two or more techniques, such as functional neuroimaging and electrophysiology. For example, functional neuroimaging can be performed to image neural activity in one or more regions of interest in a brain of a subject. In addition, electrophysiology measurements may be made on one or more neurons in the same brain regions of interest. First, functional neuroimaging data of brain neural activity is fit to a dynamic causal model (DCM), which is used to calculate neural inter-regional and intra-regional connectivity strength estimates. Next, a biophysics model is constructed using the inter-regional and intra-regional connectivity strength estimates calculated from the DCM analysis. This biophysics model is used to simulate synthetic electrophysiology data, which can be compared to the experimental electrophysiology data that was acquired for the neurons in the brain regions of interest. The biophysical model can be iteratively adjusted until model convergence is reached with the experimental electrophysiology data. In this method, sequential model fitting is used to improve modeling accuracy to generate a more accurate and comprehensive model of brain neuronal circuitry.

Exemplary functional neuroimaging techniques that can be used in the practice of the subject methods include, without limitation, functional magnetic resonance imaging (fMRI), positron emission tomography (PET), functional near-infrared spectroscopy (fNIRS), single-photon emission computed tomography (SPECT), and functional ultrasound imaging (fUS). These functional neuroimaging techniques measure localized changes in cerebral blood flow and changes in the composition of blood related to neural activity. Functional neuroimaging is useful for noninvasively detecting patterns of brain activity associated with specific stimuli or tasks.

In some embodiments, fMRI is used to monitor temporal changes in blood flow associated with changes in levels of brain activity. Blood flow increases upon neuronal activation when a region of the brain is in use. Changes in brain activity can be imaged using fMRI by detection of blood oxygen-level dependent (BOLD) signals. For a description of fMRI and methods of using fMRI for imaging of brain activity, see, e.g., Ogawa, et al. (1990) Magnetic Resonance in Medicine 14 (1): 68-78, Kim et al. (2002) Curr Opin Neurobiol. 12 (5): 607-15, Kim et al. (2012) J Cereb Blood Flow Metab. 32 (7): 1188-206, Bandettini (2012) Neuroimage 62 (2): 575-88; Zarghami et al. (2020) Neuroimage 207:116453; Logothetis, N. K. (Jun. 12, 2008), Logothetis et al. (2008) Nature 453 (7197): 869-78, and Logothetis et al. (2001) Nature. 412 (6843): 150-157; herein incorporated by reference.

Electrophysiology techniques are used to measure electrical properties in the brain, typically voltage or current changes of neurons. Exemplary electrophysiology techniques that can be used in the practice of the subject methods include, without limitation, electroencephalography (EEG), magnetoencephalography (MEG), and patch-clamping. These electrophysiology techniques are useful for identifying the specific types of neurons involved in neural networks and measuring neuron-specific changes in activity associated with brain responses.

In some embodiments, EEG is used to record neuronal electrical activity in the brain. EEG can be performed noninvasively with electrodes placed on the scalp. EEG measurements have the advantage of having high temporal resolution and can detect changes in electrical activity in the brain on a millisecond time scale. For a description of EEG and methods of using EEG for recording electrical activity in the brain, see, e.g., Niedermeyer et al. (2004) Electroencephalography: Basic Principles, Clinical Applications, and Related Fields. Lippincott Williams & Wilkins; Jackson et al. (2014) Psychophysiology 51 (11): 1061-71, Khanna et al. (2015) Neurosci Biobehav Rev. 49:105-13, Feyissa et al. (2019) Handb Clin Neurol. 160:103-124, Beres et al. (2017) Appl Psychophysiol Biofeedback 42 (4): 247-255; herein incorporated by reference.

In some embodiments, MEG is used to record magnetic fields produced by electrical currents generated in the brain. MEG detects weak magnetic fields produced by synchronized neuronal currents (i.e., ionic currents flowing in the dendrites of neurons during synaptic transmission). These weak magnetic fields can be detected using a magnetometer such as a superconducting quantum unit interference device (SQUID) or a spin exchange relaxation-free (SERF) magnetometer. For a description of MEG and methods of using MEG for recording magnetic fields associated with neuronal activity in the brain, see, e.g., Hamalainen et al. (1993) Reviews of Modern Physics. 65 (2): 413-497, Baillet et al. (2017) Nat Neurosci. 20 (3): 327-339, Gross et al. (2019) Neuron 104 (2): 189-204, Stapleton-Kotloski et al. (2018) Brain Sci. 8 (8): 157; herein incorporated by reference.

Dynamic causal modeling (DCM) is a Bayesian generative modeling paradigm that can model the causal relations in the brain taking into account neuronal dynamics, modulatory inputs, and observational data (e.g., BOLD fMRI, EEG, or MEG). DCM can be used to estimate coupling among brain regions and quantify effective connectivity strengths among neuronal populations in one or more regions of the brain. A dynamic causal model of interacting neural populations can be used to estimate neural inter-regional and intra-regional connectivity strengths from functional neuroimaging data using Bayesian statistical methods. The model may include interactions between excitatory and inhibitory neural populations. Different types of DCM can be used in calculating the connectivity strength estimates. Classical DCM assumes no state noise whereas stochastic DCM (sDCM) utilizes state noise to account for uncertainty due to neighboring regions. Spectral DCM is another paradigm that assumes state noise, but in this approach, model fitting is performed over the cross spectra of the system, which makes sDCM useful for resting state modeling. Dynamic effective connectivity (or dynamic DCM) is another approach that is useful for investigating temporal changes in connectivity strengths. For a description of different types of DCM approaches, see, e.g., Friston et al. (2003) Neuroimage 19 (4): 1273-1302, Friston et al. (2014) NeuroImage 94:396-407, Park et al. (2018) Neuroimage 180:594-608, Li et al. (2011) NeuroImage 58 (2): 442-457, Stephan et al. (2010) NeuroImage 49 (4): 3099-3109, Stephan et al. (2008) NeuroImage. 42 (2): 649-662, Marreiros et al. NeuroImage 39 (1): 269-278, Razi et al. (2015) NeuroImage 106:1-14, and Lee et al. (2006) NeuroImage. 30 (4): 1243-1254; herein incorporated by reference.

Biophysics modeling is based on the biophysics attributes of neurons and brain regions. A biophysics neuronal model may include various biophysics attributes including, but not limited to, firing patterns of individual neurons, neuronal types, receptors, inputs and outputs, topological structures, and self-connectivity. The present method utilizes a biophysics model to simulate synthetic electrophysiology data using the inter-regional and intra-regional connectivity strength estimates from dynamic causal modeling. Next, the synthetic electrophysiology data is compared to the experimental electrophysiology data acquired from the subject, and the model is improved by adjusting it iteratively to reach convergence. For a description of biophysics modeling, see, e.g., Example 5, Hill and Tononi (2005) Journal of Neurophysiology 93 (3): 1671-1698, and Murray et al. (2018) Biol Psychiatry Cogn Neurosci Neuroimaging 3 (9): 777-787; herein incorporated by reference.

In some embodiments, functional neuroimaging and electrophysiology techniques are used in combination to detect brain responses when a subject is exposed to stimuli or performing tasks. Additionally, functional neuroimaging and electrophysiology measurements of brain activity can be taken while the subject is in a resting state (e.g., absence of stimulus or taskless) to allow brain activity to be compared to a subject's “baseline” brain state, i.e., to identify brain regions exhibiting changes in neural activity associated with specific stimuli or tasks.

In some embodiments, the methods described herein are used to evaluate changes in brain function in response to optogenetic perturbation of neural activity. In certain embodiments, optogenetics is used to induce cell-specific perturbations in the brain. For example, optogenetics can be used to excite or inhibit one or more selected neurons of interest using light. For a description of optogenetics techniques, see, e.g., Abe et al., 2012; Desai et al., 2011; Duffy et al., 2015; Gerits et al., 2012; Kahn et al., 2013; Lee et al., 2010; Liu et al., 2015; Ohayon et al., 2013; Weitz et al., 2015; Weitz and Lee, 2013; herein incorporated by reference.

The methods described herein can also be used to evaluate changes in brain function in response to brain stimulation with electrical currents or magnetic fields that are applied to a selected brain area. For example, electrical brain stimulation (EBS) can be used to stimulate a neuron or neural network in the brain through the direct or indirect excitation of its cell membrane by using an electric current. For a description of EBS techniques, see, e.g., Aum et al. (2018) Front Biosci (Landmark Ed) 23:162-182, Tellez-Zenteno et al. (2011) Neurosurg Clin N Am. 22 (4): 465-75, Padberg et al. (2009) Exp. Neurol. 219:2-13, Nahas et al. (2010) Biol. Psychiatry 67:101-109, Lefaucheur et al. (2010) Exp. Neurol. 223:609-614, Levy et al. (2008) J. Neurosurg. 108:707-714, Hanajima et al. (2002) Clin. Neurophysiol. 113:635-641, Picillo et al. (2015) Brain Stimul. 8:840-842., Canavero (2014) Textbook of Cortical Brain Stimulation. Berlin: De Gruyter Open; herein incorporated by reference. Alternatively, transcranial magnetic stimulation (TMS) can be used to electrically stimulate the brain by electromagnetic induction and can be used to noninvasively stimulate specific regions of the brain. For a description of TMS techniques, see, e.g., Klomjai et al. (2015) Ann Phys Rehabil Med. 58 (4): 208-213, Lefaucheur (2019) Handb Clin Neurol. 160:559-580, Burke et al. (2019) Handb Clin Neurol. 163:73-92; herein incorporated by reference.

The subject methods are applicable to modeling the effects of neuromodulation (e.g., delivered optogenetically or by electrical stimulation) to produce single-cell-spiking level models that match experimental data (see Examples). Utilizing such models, one can test virtual neurostimulation (deep brain stimulation, optogenetic stimulation, or other neuromodulation with different combinations of parameters and targets to allow efficient optimization of stimulation parameters and targets.

### System and Computer Implemented Methods for Modeling Propagation of β-Band Oscillations

In one aspect, a computer implemented method is provided for modeling propagation of β-band oscillations and response to neuromodulation using a combination of dynamic causal modeling and biophysics modeling. The computer performs steps comprising: a) receiving functional magnetic resonance imaging data of neural activity before optogenetic stimulation and during optogenetic stimulation of D1-medium spiny neurons (D1-MSNs) and D2-medium spiny neurons (D2-MSNs) in a caudate putamen (CPu) region, an external globus pallidus (GPe) region, an internal globus pallidus (GPi) region, a subthalamic nucleus (STN) region, a substantia nigra pars reticulata (SNr) region, a thalamus (THL) region, and a motor cortex (MCX) region of the brain of the subject; b) performing spectral dynamic causal modeling of effective connectivity strengths among the CPu region, the GPe region, the GPi region, the STN region, the SNr region, the THL region, and the MCX region; c) receiving experimental electrophysiological data for the CPu region; d) estimating effective connectivity strengths of GABAergic connections using the experimental electrophysiological data for the CPu region; e) estimating effective connectivity strengths of glutamatergic connections using dynamic causal modeling of the functional magnetic resonance imaging data; f) performing biophysics modeling using a Hodgkin-Huxley model to generate simulated electrophysiology data using the effective connectivity strength estimates; g) optimizing GABAergic projections iteratively until the simulated electrophysiology data matches the experimental electrophysiological data for the CPu region; h) calculating a temporal profile of average power of beta-band frequencies for each neuron in the CPu region, the GPe region, the GPi region, the STN region, the SNr region, the THL region, and the MCX region; and i) comparing total amount of co-occurred beta-band oscillation power before optogenetic stimulation to co-occurred beta-band oscillation power during optogenetic stimulation to model the propagation of β-band oscillations in the brain of the subject.

In certain embodiments, the D1-MSN are Huxley-Hudgkin neurons.

In certain embodiments, the experimental electrophysiology data comprise single-neuron recordings.

In certain embodiments, the single-neuron recordings are from GABAergic neurons of the CPu-region.

In certain embodiments, the glutamatergic connections are modeled as 1-to-1 connections with connection strengths proportional to effective connectivity estimated by the DCM.

In certain embodiments, the CPu-GPi/GPe and GPi/SNr-thalamus GABAergic projections are modeled as 1-to-n diffusive projections with connectivity strength wGABA, where n and wGABA are free parameters, wherein N and wGABA are searched across parameter space until the simulated spike rates match statistically with the single-neuron recordings.

In certain embodiments, the optogenetic stimulation comprises optogenetically inhibiting the D1-MSNs in the GPi region of the brain of the subject.

In certain embodiments, optogenetically inhibiting the D1-MSNs comprises sustained shunting inhibition of the D1-MSNs in the GPi region of the brain of the subject.

In certain embodiments, the optogenetic stimulation comprises optogenetically inhibiting the D2-MSNs in the GPe region of the brain of the subject.

In certain embodiments, optogenetically inhibiting the D2-MSNs comprises sustained shunting inhibition of the D2-MSNs in the GPe region of the brain of the subject.

In certain embodiments, the optogenetic stimulation comprises direct activation of the D1-MSNs or the D2-MSNs with a strength of 500 pA, 700 pA, or 900 pA.

In certain embodiments, the optogenetic stimulation comprises optogenetically stimulating the D1-MSNs periodically in the Gpe region of the brain of the subject.

In certain embodiments, the optogenetic stimulation comprises optogenetically stimulating the D1-MSNs and the D2-MSNs randomly in the Gpe region of the brain of the subject using a plurality of stimulation sequences with randomly spaced pulses, wherein each neuron is stimulated with one of the stimulation sequences such that synchronized neurons are decoupled.

In certain embodiments, the optogenetic stimulation is performed with periodic stimulation at a frequency of 130 Hz.

In some embodiments, a non-human animal is used to develop the simulation. The non-human animal may include, but is not limited to, non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, and geese. β-band oscillations can be generated in the brain of a non-human animal, for example, by optogenetic stimulation of D1-MSNs and/or D2-MSNs in the striatum at β-band frequencies in the range of 12.5 Hz to 30 Hz. In some embodiments, β-band oscillations are generated in the brain of a non-human animal by optogenetic stimulation of D1-MSNs and/or D2-MSNs in the striatum at a frequency of 20 Hz. Functional magnetic resonance imaging data and experimental electrophysiology data can be obtained from such animals for use in the methods described herein for modeling propagation of β-band oscillations and response to neuromodulation.

The methods can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, a system for performing the computer implemented method, as described, may include a processor, a storage component (i.e., memory), a display component, and other components typically present in general purpose computers. In some embodiments, the processor is provided by a computer or handheld device (e.g., a cell phone or tablet). The storage component stores information accessible by the processor, including instructions that may be executed by the processor and data that may be retrieved, manipulated or stored by the processor.

The storage component includes instructions. For example, the storage component includes instructions for modeling propagation of β-band oscillations and response to neuromodulation according to the methods described herein. The computer processor is coupled to the storage component and configured to execute the instructions stored in the storage component in order to receive functional magnetic resonance imaging data and experimental electrophysiology data and analyze the data according to one or more algorithms, as described herein.

The processor and/or memory may be operably connected to a display device, for example, via a wired, such as a Universal Serial Bus (USB) connection, or wireless connection, such as a Bluetooth connection. Any convenient display device, such as a liquid crystal display (LCD), light-emitting diode (LED) display, plasma (PDP) display, quantum dot (QLED) display or cathode ray tube display device may be used. The display component displays information regarding the locations of the pathological protein aggregates in the brain of the subject. In some embodiments, the display displays information regarding the propagation of β-band oscillations in the brain of the subject (or suppression thereof by neuromodulation), as determined by the computer implemented method.

The storage component may be of any type capable of storing information accessible by the processor, such as a hard-drive, memory card, ROM, RAM, DVD, CD-ROM, USB Flash drive, write-capable, and read-only memories. The processor may be a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

In some embodiments, the method can be performed using a cloud computing system. In these embodiments, images of the subject's brain can be exported to a cloud computer, which runs the program, and returns an output to the user.

Components of systems for carrying out the presently disclosed methods are further described in the examples below.

### Neuromodulation Therapy for Treatment of Parkinson's Disease

In some embodiments, neuromodulation is used to suppress β-band oscillations in the brain of a subject to treat Parkinson's disease. Neuromodulation can be achieved using electrical stimulation (e.g., from implanted, clinically approved electrodes), transcranial magnetic stimulation, transcranial electrical stimulation, or focused ultrasound, among other techniques. For electrical stimulation, deep brain stimulators can be used. In some embodiments, cortical layers or cell-types are targeted specifically with genetically encodable modulation techniques, such as optogenetics.

### Optogenetics

In some embodiments, optogenetics is used in a manner effective to suppress β-band oscillations in the brain of a subject to treat Parkinson's disease. Optogenetics is used to allow optical control of activation (i.e., depolarization) or inhibition (i.e., hyperpolarization) of neurons that have been genetically modified to express light-responsive ion channels. In some embodiments, the light-responsive ion channel is a naturally occurring or synthetic opsin that uses a retinal-based cofactor (e.g., all-trans retinal for the microbial opsins) to respond to light. For example, light-responsive cation-conducting opsins (e.g., channelrhodopsin that conducts Ca²⁺) can be used to activate or depolarize neurons. Light-responsive anion-conducting opsins (e.g., channelrhodopsin or halorhodopsin that conduct chloride ions) or light-responsive proton conductance regulators (e.g., bacteriorhodopsin or archaerhodopsin) can be used to inhibit or hyperpolarize neurons. The levels of retinoids present in a mammalian brain are usually sufficient for expressed opsins to function without supplementation of cofactors. For a description of optogenetics and its use in controlling neural activity, see, e.g., Aravanis et al. (2007) J Neural Eng 4: S143-S156, Arenkiel et al. (2007) Neuron 54:205-218, Boyden et al. (2005) Nat Neurosci 8:1263-1268, Chow et al. (2010) Nature 463:98-10, Gradinaru et al. (2007) J Neurosci 27:14231-14238, Gradinaru et al. (2008) Brain Cell Biol 36:129-139, Gradinaru et al. (2010) Cell 141:1-12, Li et al. (2005) Proc Natl Acad Sci 102:17816-17821, Lin et al. 2009. Characterization of engineered channelrhodopsin variants with improved properties and kinetics. Biophys J 96:1803-1814, Yizhar et al. (2011) Microbial opsins: A family of single-component tools for optical control of neural activity. Cold Spring Harbor Protoc, Zhang et al. (2007) Nat Methods 4:139-141, Zhang et al. (2006) Nat Methods 3:785-792, Zhang et al. (2007) Nature 446:633-639, Zhang et al. (2008) Nat Neurosci 11:631-633; and U.S. Pat. Nos. 10,914,803; 10,589,123; 10,583,309; 10,568,516; 10,568,307; 10,538,560; 10,478,499; 10,220,092; 10,196,431; 10,087,223; 10,052,383; 9,969,783; 9,878,176; 9,855,442; 9,757,587; 9,458,208; and 8,834,546; herein incorporated by reference in their entireties.

In some embodiments, a target neuron is genetically modified to express a light-responsive ion channel that, when stimulated by an appropriate light stimulus, hyperpolarizes or depolarizes the stimulated target neuron. The term “genetic modification” refers to a permanent or transient genetic change induced in a cell following introduction into the cell of a heterologous nucleic acid (i.e., nucleic acid exogenous to the cell). Genetic change (“modification”) can be accomplished by incorporation of the heterologous nucleic acid into the genome of the host cell, or by transient or stable maintenance of the heterologous nucleic acid as an extrachromosomal element. Where the cell is a eukaryotic cell, a permanent genetic change can be achieved by introduction of the nucleic acid into the genome of the cell. Suitable methods of genetic modification include the use of viral infection, transfection, conjugation, protoplast fusion, electroporation, particle gun technology, calcium phosphate precipitation, direct microinjection, and the like.

In some cases, a target cell that expresses a light-responsive polypeptide can be activated or inhibited upon exposure to light of varying wavelengths. In some cases, a target cell that expresses a light-responsive polypeptide is a neuronal cell that expresses a light-responsive polypeptide, and exposure to light of varying wavelengths results in depolarization or polarization of the neuron.

In some instances, the light-responsive polypeptide is a light-responsive ion channel polypeptide. The light-responsive ion channel polypeptides are adapted to allow one or more ions to pass through the plasma membrane of a target cell when the polypeptide is illuminated with light of an activating wavelength. Light-responsive proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-responsive polypeptide depolarizes the excitable cell when activated by light of an activating wavelength. In some embodiments, the light-responsive polypeptide hyperpolarizes the excitable cell when activated by light of an activating wavelength.

In some cases, a light-responsive polypeptide mediates a hyperpolarizing current in the target cell it is expressed in when the cell is illuminated with light. Non-limiting examples of light-responsive polypeptides capable of mediating a hyperpolarizing current can be found, e.g., in U.S. Pat. Nos. 9,359,449 and 9,175,095. Non-limiting examples of hyperpolarizing light-responsive polypeptides include NpHr, eNpHr2.0, eNpHr3.0, eNpHr3.1 or GtR3. In some cases, a light-responsive polypeptide mediates a depolarizing current in the target cell it is expressed in when the cell is illuminated with light. Non-limiting examples of depolarizing light-responsive polypeptides include “C1V1”, ChR1, VChR1, ChR2. Additional information regarding other light-responsive cation channels, anion pumps, and proton pumps can be found in U.S. Patent Application Publication No: 2009/0093403; and U.S. Pat. No. 9,359,449.

In some embodiments, the light-responsive polypeptide can be activated by blue light (e.g., in range of 490 nm-450 nm). In one embodiment, the light-responsive polypeptide can be activated by light having a wavelength of about 473 nm. In some embodiments, the light-responsive polypeptide can be activated by yellow light (e.g., in range of 590 nm-560 nm). In another embodiment, the light-responsive polypeptide can be activated by light having a wavelength of about 560 nm. In another embodiment, the light-responsive polypeptide can be activated by red light (e.g., in range of 700 nm-635 nm). In another embodiment, the light-responsive polypeptide can be activated by light having a wavelength of about 630 nm. In other embodiments, the light-responsive polypeptide can be activated by violet light (e.g., in range of 450 nm-400 nm). In one embodiment, light-responsive polypeptide can be activated by light having a wavelength of about 405 nm. In other embodiments, the light-responsive polypeptide can be activated by green light (e.g., in range of 560 nm-520 nm). In other embodiments, the light-responsive polypeptide can be activated by cyan light (e.g., in range of 520 nm-490 nm). In other embodiments, the light-responsive polypeptide can be activated by orange light (e.g., in range of 635 nm-590 nm). A person of skill in the art would recognize that each light-responsive polypeptide will have its own range of activating wavelengths.

In some cases, the regions of the brain with neurons containing a light-responsive polypeptide are illuminated using one or more optical fibers. The optical fiber may be configured in any suitable manner to direct a light emitted from a suitable source of light, e.g., a laser or light-emitting diode (LED) light source, to the region of the brain. The optical fiber may be any suitable optical fiber. In some cases, the optical fiber is a multimode optical fiber. The optical fiber may include a core defining a core diameter, where light from the light source passes through the core. The optical fiber may have any suitable core diameter. In some cases, the core diameter of the optical fiber is 10 mm or more, e.g., 20 mm or more, 30 mm or more, 40 mm or more, 50 mm or more, 60 mm or more, including 80 mm or more, and is 1,000 mm or less, e.g., 500 mm or less, 200 mm or less, 100 mm or less, including 70 mm or less. In some embodiments, the core diameter of the optical fiber is in the range of 10 to 1,000 mm, e.g., 20 to 500 mm, 30 to 200 mm, including 40 to 100 mm.

The optical fiber end that is implanted into the target region of the brain may have any suitable configuration suitable for illuminating a region of the brain with a light stimulus delivered through the optical fiber. In some cases, the optical fiber includes an attachment device at or near the distal end of the optical fiber, where the distal end of the optical fiber corresponds to the end inserted into the subject. In some cases, the attachment device is configured to connect to the optical fiber and facilitate attachment of the optical fiber to the subject, such as to the skull of the subject. Any suitable attachment device may be used. In some cases, the attachment device includes a ferrule, e.g., a metal, ceramic or plastic ferrule. The ferrule may have any suitable dimensions for holding and attaching the optical fiber.

In certain embodiments, methods of the present disclosure may be performed using any suitable electronic components to control and/or coordinate the various optical components used to illuminate the regions of the brain. The optical components (e.g., light source, optical fiber, lens, objective, mirror, and the like) may be controlled by a controller, e.g., to coordinate the light source illuminating the regions of the brain with light pulses. The controller may include a driver for the light source that controls one or more parameters associated with the light pulses, such as, but not limited to the frequency, pulse width, duty cycle, wavelength, intensity, etc. of the light pulses. The controllers may be in communication with components of the light source (e.g., collimators, shutters, filter wheels, moveable mirrors, lenses, etc.).

In some embodiments, the light-responsive polypeptides are activated by light pulses that can have a duration for any of about 1 millisecond (ms), about 2 ms, about 3, ms, about 4, ms, about 5 ms, about 6 ms, about 7 ms, about 8 ms, about 9 ms, about 10 ms, about 15 ms, about 20 ms, about 25 ms, about 30 ms, about 35 ms, about 40 ms, about 45 ms, about 50 ms, about 60 ms, about 70 ms, about 80 ms, about 90 ms, about 100 ms, about 200 ms, about 300 ms, about 400 ms, about 500 ms, about 600 ms, about 700 ms, about 800 ms, about 900 ms, about 1 sec, about 1.25 sec, about 1.5 sec, or about 2 sec, inclusive, including any times in between these numbers. In some embodiments, the light-responsive polypeptides are activated by light pulses that can have a light power density of any of about 0.05 mW/mm², about 0.1 mW/mm², about 0.25 mW/mm², about 0.5 mW/mm², about 0.75 mW/mm², about 1 mW/mm², about 2 mW/mm², about 3 mW/mm², about 4 mW/mm², about 5 mW/mm², about 6 mW/mm², about 7 mW/mm², about 8 mW/mm², about 9 mW/mm², about 10 mW/mm², about 20 mW/mm², about 50 mW/mm², about 100 mW/mm², about 250 mW/mm², about 500 mW/mm², about 750 mW/mm², about 1000 mW/mm², about 1100 mW/mm², about 1200 mW/mm², about 1300 mW/mm², about 1400 mW/mm², about 1500 mW/mm², about 1600 mW/mm², about 1700 mW/mm², about 1800 mW/mm², about 1900 mW/mm², about 2000 mW/mm², about 2100 mW/mm², about 2200 mW/mm², about 2300 mW/mm², about 2400 mW/mm², about 2500 mW/mm², about 2600 mW/mm², about 2700 mW/mm², about 2800 mW/mm², about 2900 mW/mm², about 3000 mW/mm², about 3100 mW/mm², about 3100 mW/mm², about 3300 mW/mm², about 3400 mW/mm², or about 3500 mW/mm², inclusive, including any values between these numbers.

The light stimulus used to activate the light-responsive polypeptide may include light pulses characterized by, e.g., frequency, pulse width, duty cycle, wavelength, intensity, etc. In some cases, the light stimulus includes two or more different sets of light pulses, where each set of light pulses is characterized by different temporal patterns of light pulses. The temporal pattern may be characterized by any suitable parameter, including, but not limited to, frequency, period (i.e., total duration of the light stimulus), pulse width, duty cycle, etc.

The light pulses may have any suitable frequency. In some cases, the set of light pulses contains a single pulse of light that is sustained throughout the duration of the light stimulus. In some cases, the light pulses of a set have a frequency of 0.1 Hz or more, e.g., 0.5 Hz or more, 1 Hz or more, 5 Hz or more, 10 Hz or more, 20 Hz or more, 30 Hz or more, 40 H or more, including 50 Hz or more, or 60 Hz or more, or 70 Hz or more, or 80 Hz or more, or 90 Hz or more, or 100 Hz or more, and have a frequency of 100,000 Hz or less, e.g., 10,000 Hz or less, 1,000 Hz or less, 500 Hz or less, 400 Hz or less, 300 Hz or less, 200 Hz or less, including 100 Hz or less. In some embodiments, the light pulses have a frequency in the range of 0.1 to 100,000 Hz, e.g., 1 to 10,000 Hz, 1 to 1,000 Hz, including 5 to 500 Hz, or 10 to 100 Hz.

In some cases, the two sets of light pulses are characterized by having different parameter values, such as different pulse widths, e.g., short or long. The light pulses may have any suitable pulse width. In some cases, the pulse width is 0.1 ms or longer, e.g., 0.5 ms or longer, 1 ms or longer, 3 ms or longer, 5 ms or longer, 7.5 ms or longer, 10 ms or longer, including 15 ms or longer, or 20 ms or longer, or 25 ms or longer, or 30 ms or longer, or 35 ms or longer, or 40 ms or longer, or 45 ms or longer, or 50 ms or longer, and is 500 ms or shorter, e.g., 100 ms or shorter, 90 ms or shorter, 80 ms or shorter, 70 ms or shorter, 60 ms or shorter, 50 ms or shorter, 45 ms or shorter, 40 ms or shorter, 35 ms or shorter, 30 ms or shorter, 25 ms or shorter, including 20 ms or shorter. In some embodiments, the pulse width is in the range of 0.1 to 500 ms, e.g., 0.5 to 100 ms, 1 to 80 ms, including 1 to 60 ms, or 1 to 50 ms, or 1 to 30 ms.

The average power of the light pulse, measured at the tip of an optical fiber delivering the light pulse to regions of the brain, may be any suitable power. In some cases, the power is 0.1 mW or more, e.g., 0.5 mW or more, 1 mW or more, 1.5 mW or more, including 2 mW or more, or 2.5 mW or more, or 3 mW or more, or 3.5 mW or more, or 4 mW or more, or 4.5 mW or more, or 5 mW or more, and may be 1,000 mW or less, e.g., 500 mW or less, 250 mW or less, 100 mW or less, 50 mW or less, 40 mW or less, 30 mW or less, 20 mW or less, 15 mW or less, including 10 mW or less, or 5 mW or less. In some embodiments, the power is in the range of 0.1 to 1,000 mW, e.g., 0.5 to 100 mW, 0.5 to 50 mW, 1 to 20 mW, including 1 to 10 mW, or 1 to 5 mW.

The wavelength and intensity of the light pulses may vary and may depend on the activation wavelength of the light-responsive polypeptide, optical transparency of the region of the brain, the desired volume of the brain to be illuminated, etc.

The volume of a brain region illuminated by the light pulses may be any suitable volume. In some cases, the illuminated volume is 0.001 mm³ or more, e.g., 0.005 mm³ or more, 0.001 mm³ or more, 0.005 mm³ or more, 0.01 mm³ or more, 0.05 mm³ or more, including 0.1 mm³ or more, and is 100 mm³ or less, e.g., 50 mm³ or less, 20 mm³ or less, 10 mm³ or less, 5 mm³ or less, 1 mm³ or less, including 0.1 mm³ or less. In certain cases, the illuminated volume is in the range of 0.001 to 100 mm³, e.g., 0.005 to 20 mm³, 0.01 to 10 mm³, 0.01 to 5 mm³, including 0.05 to 1 mm³.

In some embodiments, the light-responsive polypeptide expressed in a cell can be fused to one or more amino acid sequence motifs selected from the group consisting of a signal peptide, an endoplasmic reticulum (ER) export signal, a membrane trafficking signal, and/or an N-terminal golgi export signal. The one or more amino acid sequence motifs which enhance light-responsive protein transport to the plasma membranes of mammalian cells can be fused to the N-terminus, the C-terminus, or to both the N- and C-terminal ends of the light-responsive polypeptide. In some cases, the one or more amino acid sequence motifs which enhance light-responsive polypeptide transport to the plasma membranes of mammalian cells is fused internally within a light-responsive polypeptide. Optionally, the light-responsive polypeptide and the one or more amino acid sequence motifs may be separated by a linker. In some embodiments, the light-responsive polypeptide can be modified by the addition of a trafficking signal (ts) which enhances transport of the protein to the cell plasma membrane. In some embodiments, the trafficking signal can be derived from the amino acid sequence of the human inward rectifier potassium channel Kir2.1. In some embodiments, the signal peptide sequence in the protein can be deleted or substituted with a signal peptide sequence from a different protein.

Exemplary light-responsive polypeptides and amino acid sequence motifs that find use in the present system and method are disclosed in, e.g., U.S. Pat. Nos. 10,538,560; 10,568,307; 9,284,353; 9,359,449; and 9,365,628; herein incorporated by reference.

Light-responsive polypeptides of interest include, for example, a step function opsin (SFO)6 protein or a stabilized step function opsin (SSFO) protein that can have specific amino acid substitutions at key positions in the retinal binding pocket of the protein. See, for example, WO 2010/056970, the disclosure of which is hereby incorporated by reference in its entirety. The polypeptide may be a cation channel derived from Volvox carteri (VChR1), optionally comprising one or more amino acid substitutions, e.g., C123A; C123S; D151A, etc. A light-responsive cation channel protein can be a C1V1 chimeric protein derived from the VChR1 protein of Volvox carteri and the ChR1 protein from Chlamydomonas reinhardti, wherein the protein comprises the amino acid sequence of VChR1 having at least the first and second transmembrane helices replaced by the first and second transmembrane helices of ChR1, optionally having an amino acid substitution at amino acid residue E122 or E162. In other embodiments, the light-responsive cation channel protein is a C1C2 chimeric protein derived from the ChR1 and the ChR2 proteins from Chlamydomonas reinhardti, wherein the protein is responsive to light and is capable of mediating a depolarizing current in the cell when the cell is illuminated with light. In some embodiments, a depolarizing light-responsive polypeptide is a red shifted variant of a depolarizing light-responsive polypeptide derived from Chlamydomonas reinhardtii; referred to as a “ReaChR polypeptide” or “ReaChR protein” or “ReaChR.” In some embodiments, a depolarizing light-responsive polypeptide is a SdChR polypeptide derived from Scherffelia dubia, wherein the SdChR polypeptide is capable of transporting cations across a cell membrane when the cell is illuminated with light. In some embodiments, a depolarizing light-responsive polypeptide is CnChR1, derived from Chlamydomonas noctigama, wherein the CnChR1 polypeptide is capable of transporting cations across a cell membrane when the cell is illuminated with light. In some embodiments, the light-responsive cation channel protein is a CsChrimson chimeric protein derived from a CsChR protein of Chioromonas subdivisa and CnChR1 protein from Chlamydomonas noctigama, wherein the N-terminus of the protein comprises the amino acid sequence of residues 1-73 of CsChR followed by residues 79-350 of the amino acid sequence of CnChR1; is responsive to light; and is capable of mediating a depolarizing current in the cell when the cell is illuminated with light. In some embodiments, a depolarizing light-responsive polypeptide can be, e.g., ShChR1, derived from Stigeoclonium helveticum, wherein the ShChR1 polypeptide is capable of transporting cations across a cell membrane when the cell is illuminated with light.

In some embodiments, a depolarizing light-responsive polypeptide is derived from Chlamydomonas reinhardtii (CHR1, and particularly CHR2) wherein the polypeptide is capable of transporting cations across a cell membrane when the cell is illuminated with light; and is capable of mediating a depolarizing current in the cell when the cell is illuminated with light. In some embodiments CaMKIIa-driven, humanized channelrhodopsin CHR2 H134R mutant fused to EYFP is used for optogenetic activation. The light used to activate the light-responsive cation channel protein derived from Chlamydomonas reinhardtii can have a wavelength between about 460 and about 495 nm or can have a wavelength of about 480 nm. The light-responsive cation channel protein can additionally comprise substitutions, deletions, and/or insertions introduced into a native amino acid sequence to increase or decrease sensitivity to light, increase or decrease sensitivity to particular wavelengths of light, and/or increase or decrease the ability of the light-responsive cation channel protein to regulate the polarization state of the plasma membrane of the cell. Additionally, the light-responsive cation channel protein can comprise one or more conservative amino acid substitutions and/or one or more non-conservative amino acid substitutions. The light-responsive proton pump protein containing substitutions, deletions, and/or insertions introduced into the native amino acid sequence suitably retains the ability to transport cations across a cell membrane. The protein may comprise various amino acid substitutions, e.g., one or more of H134R; T159C; L132C; E123A; etc. The protein may further comprise a fluorescent protein, for example, but not limited to, a yellow fluorescent protein, a red fluorescent protein, a green fluorescent protein, or a cyan fluorescent protein.

Neurons can be selectively activated or inhibited optogenetically by engineering neurons to express one or more light-responsive polypeptides configured to hyperpolarize or depolarize the neurons. Suitable light-responsive polypeptides and methods used thereof are described further below.

A light-responsive polypeptide for use in the present disclosure may be any suitable light-responsive polypeptide for selectively activating neurons of a subtype by illuminating the neurons with an activating light stimulus. In some instances, the light-responsive polypeptide is a light-responsive ion channel polypeptide. The light-responsive ion channel polypeptides are adapted to allow one or more ions to pass through the plasma membrane of a target cell when the polypeptide is illuminated with light of an activating wavelength. Light-responsive proteins may be characterized as ion pump proteins, which facilitate the passage of a small number of ions through the plasma membrane per photon of light, or as ion channel proteins, which allow a stream of ions to freely flow through the plasma membrane when the channel is open. In some embodiments, the light-responsive polypeptide depolarizes the cell when activated by light of an activating wavelength. In some embodiments, the light-responsive polypeptide hyperpolarizes the cell when activated by light of an activating wavelength. Suitable hyperpolarizing and depolarizing polypeptides are known in the art and include, e.g., a channelrhodopsin (e.g., ChR2), variants of ChR2 (e.g., C128S, D156A, C128S+D156A, E123A, E123T), iC1C2, C1C2, GtACR2, NpHR, eNpHR3.0, C1V1, VChR1, VChR2, SwiChR, Arch, ArchT, KR2, ReaChR, ChiEF, Chronos, ChRGR, CsChrimson, and the like. In some cases, the light-responsive polypeptide includes bReaCh-ES, as described in, e.g., Rajasethupathy et al., Nature. 2015 Oct. 29; 526 (7575): 653, which is incorporated by reference. Hyperpolarizing and depolarizing opsins have been described in various publications; see, e.g., Berndt and Deisseroth (2015) Science 349:590; Berndt et al. (2014) Science 344:420; and Guru et al. (Jul. 25, 2015) Intl. J. Neuropsychopharmacol. pp. 1-8 (PMID 26209858).

The light-responsive polypeptide may be introduced into the neurons using any suitable method. In some cases, the neurons of a subtype of interest are genetically modified to express a light-responsive polypeptide. In some cases, the neurons may be genetically modified using a viral vector, e.g., an adeno-associated viral vector, containing a nucleic acid having a nucleotide sequence that encodes the light-responsive polypeptide. The viral vector may include any suitable control elements (e.g., promoters, enhancers, recombination sites, etc.) to control expression of the light-responsive polypeptide according to neuronal subtype, timing, presence of an inducer, etc.

“Operably linked” refers to a juxtaposition wherein the components so described are in a relationship permitting them to function in their intended manner. For instance, a promoter is operably linked to a nucleotide sequence (e.g., a protein coding sequence, e.g., a sequence encoding an mRNA; a non-protein coding sequence, e.g., a sequence encoding a light-reactive protein; and the like) if the promoter affects its transcription and/or expression.

Neuron-specific promoters and other control elements (e.g., enhancers) are known in the art. Suitable neuron-specific control sequences include, but are not limited to, a neuron-specific enolase (NSE) promoter (see, e.g., EMBL HSENO2, X51956; see also, e.g., U.S. Pat. Nos. 6,649,811, 5,387,742); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn et al. (2010) Nat. Med. 16:1161); a serotonin receptor promoter (see, e.g., GenBank S62283); a tyrosine hydroxylase promoter (TH) (see, e.g., Nucl. Acids. Res. 15:2363-2384 (1987) and Neuron 6:583-594 (1991)); a GnRH promoter (see, e.g., Radovick et al., Proc. Natl. Acad. Sci. USA 88:3402-3406 (1991)); an L7 promoter (see, e.g., Oberdick et al., Science 248:223-226 (1990)); a DNMT promoter (see, e.g., Bartge et al., Proc. Natl. Acad. Sci. USA 85:3648-3652 (1988)); an enkephalin promoter (see, e.g., Comb et al., EMBO J. 17:3793-3805 (1988)); a myelin basic protein (MBP) promoter; a CMV enhancer/platelet-derived growth factor-.beta. promoter (see, e.g., Liu et al. (2620) Gene Therapy 11:52-60); a motor neuron-specific gene Hb9 promoter (see, e.g., U.S. Pat. No. 7,632,679; and Lee et al. (2620) Development 131:3295-3306); and an alpha subunit of Ca²⁺-calmodulin-dependent protein kinase II (CaMKII) promoter (see, e.g., Mayford et al. (1996) Proc. Natl. Acad. Sci. USA 93:13250). Other suitable promoters include elongation factor (EF) 1 and dopamine transporter (DAT) promoters.

In some cases, neuronal subtype-specific expression of the light-responsive polypeptide may be achieved by using recombination systems, e.g., Cre-Lox recombination, Flp-FRT recombination, etc. Cell type-specific expression of genes using recombination has been described in, e.g., Fenno et al., Nat Methods, 2014 July; 11 (7): 763; and Gompf et al., Front Behav Neurosci. 2015 Jul. 2; 9:152, which are incorporated by reference herein.

In some embodiments, the vector is a recombinant adeno-associated virus (AAV) vector. AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The application of AAV as a vector for gene therapy has been rapidly developed in recent years. Wild-type AAV could infect, with a comparatively high titer, dividing or non-dividing cells, or tissues of mammal, including human, and also can integrate into in human cells at specific site (on the long arm of chromosome 19) (Kotin et al, Proc. Natl. Acad. Sci. U.S.A., 1990. 87:2211-2215; Samulski et al, EMBO J., 1991. 10:3941-3950 the disclosures of which are hereby incorporated by reference herein in their entireties). AAV vector without the rep and cap genes loses specificity of site-specific integration, but may still mediate long-term stable expression of exogenous genes. AAV vector exists in cells in two forms, wherein one is episomic outside of the chromosome; another is integrated into the chromosome, with the former as the major form. Moreover, AAV has not hitherto been found to be associated with any human disease, nor any change of biological characteristics arising from the integration has been observed. There are sixteen serotypes of AAV reported in literature, respectively named AAV1, AAV2, AAV3, AAV4, AAV5, AAV6, AAV7, AAV8, AAV9, AAV10, AAV11, AAV12, AAV13, AAV14, AAV15, and AAV16, wherein AAV5 is originally isolated from humans (Bantel-Schaal, and H. zur Hausen. Virology, 1984. 134:52-63), while AAV1-4 and AAV6 are all found in the study of adenovirus (Ursula Bantel-Schaal, Hajo Delius and Harald zur Hausen. J. Virol., 1999. 73:939-947).

AAV vectors may be prepared using any convenient methods. Adeno-associated viruses of any serotype are suitable (See, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” In Parvoviruses (J R Kerr, S F Cotmore. M E Bloom, R M Linden, C R Parrish, Eds.) p 5-14, Hudder Arnold, London, U K (2006); and D E Bowles, J E Rabinowitz, R J Samulski “The Genus Dependovirus” (J R Kerr, S F Cotmore. M E Bloom, R M Linden, C R Parrish, Eds.) p 15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6,995,006 and WO/1999/011764 titled “Methods for Generating High Titer Helper-free Preparation of Recombinant AAV Vectors”, the disclosures of which are herein incorporated by reference in their entirety. Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: 91/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some embodiments, the vector(s) for use in the methods of the invention are encapsidated into a virus particle (e.g., AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, and AAV16). Accordingly, the invention includes a recombinant virus particle (recombinant because it contains a recombinant polynucleotide) comprising any of the vectors described herein. Methods of producing such particles are known in the art and are described in U.S. Pat. No. 6,596,535.

It is understood that one or more vectors may be administered to neural cells. If more than one vector is used, it is understood that they may be administered at the same or at different times.

In certain embodiments, optogenetic neuromodulation of medium spiny neurons is performed according to a method comprising: (a) optogenetically inhibiting D1-medium spiny neurons (D1-MSNs) in a globus pallidus internal (GPi) region of the brain of the subject, wherein D1-MSN mediated β-band oscillations are suppressed; (b) optogenetically inhibiting D2-medium spiny neurons (D2-MSNs) in a globus pallidus external (Gpe) region of the brain of the subject, wherein D2-MSN mediated-band oscillations are suppressed; (c) optogenetically stimulating the D1-MSNs periodically in the Gpe region of the brain of the subject, wherein D1-MSN mediated β-band oscillations are suppressed; or (d) optogenetically stimulating the D1-MSNs and the D2-MSNs randomly in the Gpe region of the brain of the subject, wherein D1-MSN mediated β-band oscillations and D2-MSN mediated β-band oscillations are suppressed; or any combination of (a)-(d).

In certain embodiments, the method comprises: (a) optogenetically inhibiting the D1-MSNs in the GPi region of the brain of the subject, wherein the D1-MSN mediated β-band oscillations are suppressed; (b) optogenetically inhibiting the D2-MSNs in the Gpe region of the brain of the subject, wherein the D2-MSN mediated β-band oscillations are suppressed; (c) optogenetically stimulating the D1-MSNs periodically in the Gpe region of the brain of the subject, wherein the D1-MSN mediated β-band oscillations are suppressed; and (d) optogenetically stimulating the D1-MSNs and the D2-MSNs randomly in the Gpe region of the brain of the subject, wherein the D1-MSN mediated β-band oscillations and the D2-MSN mediated β-band oscillations are suppressed.

In certain embodiments, optogenetically inhibiting the D1-MSNs or the D2-MSNs in the Gpi region or the Gpe region comprises: introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and illuminating the light-responsive ion channel with light at a wavelength that activates the light-responsive ion channel, wherein conduction of ions by the light-responsive ion channel in response to absorption of light results in hyperpolarization and inhibition of the D1-MSNs or the D2-MSNs.

In certain embodiments, the light-responsive ion channel is a light-responsive anion-conducting opsin or a light-responsive proton conductance regulator. In certain embodiments, the light-responsive anion-conducting opsin conducts chloride ions (Cl⁻). In certain embodiments, the anion-conducting opsin is an anion-conducting channelrhodopsin or halorhodopsin. In certain embodiments, the halorhodopsin is a Natronomonas pharaonis halorhodopsin (NpHR), enhanced NpHR (eNpHR) 1.0, eNpHR 2.0, or eNpHR 3.0. In certain embodiments, the anion-conducting channelrhodopsin is iC1C2, SwiChR, SwiChR++, or iC++. In certain embodiments, the light-responsive proton conductance regulator is a bacteriorhodopsin or an archaerhodopsin. In certain embodiments, the light-responsive proton conductance regulator is Arch from Halorubrum sodomense, ArchT from Halorubrum sp., TP009 from Leptosphaeria maculans, or Mac from Leptosphaeria maculans.

In certain embodiments, optogenetically stimulating the D1-MSNs or the D2-MSNs in the Gpi region or the Gpe region comprises: introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and illuminating the light-responsive ion channel with light at a wavelength that activates the light-responsive ion channel, wherein conduction of ions by the light-responsive ion channel in response to absorption of light results in depolarization and activation of the D1-MSNs or the D2-MSNs. In certain embodiments, the light-responsive ion channel is a light-responsive cation-conducting opsin. In certain embodiments, the light-responsive cation-conducting opsin conducts calcium cations (Ca²⁺). In certain embodiments, the light-responsive cation-conducting opsin is a light-responsive cation-conducting channelrhodopsin. In certain embodiments, the light-responsive cation-conducting channelrhodopsin is a Chlamydomonas reinhardtii channelrhodopsin or a Volvox carteri channelrhodopsin. In certain embodiments, the light-responsive cation-conducting channelrhodopsin is a Chlamydomonas reinhardtii channelrhodopsin-1 (ChR1), a Chlamydomonas reinhardtii channelrhodopsin-2 (ChR2), a Volvox carteri channelrhodopsin-1 (VChR1), or a chimeric ChR1-VChR1 channelrhodopsin.

### Electrical Stimulation

In certain embodiments, electrical stimulation is applied to the brain of a subject using an electrode to treat Parkinson's disease. The method includes positioning an electrode in a region of the brain of the subject to deliver electrical stimulation to the brain to suppress β-band oscillations. In some embodiments, the method comprises positioning a first electrode at a first location in a globus pallidus internal (GPi) region of the brain of the subject to deliver electrical stimulation to D1-medium spiny neurons in the Gpi region; positioning a second electrode at a second location in a globus pallidus external (Gpe) region of the brain of the subject to deliver electrical stimulation to D1-medium spiny neurons and D2-medium spiny neurons in the Gpe region; and applying electrical stimulation to the Gpi region of the brain of the subject using the first electrode and applying electrical stimulation to the Gpe region of the brain of the subject using the second electrode in a manner effective to suppress β-band oscillations to treat Parkinson's disease.

As used herein, the phrases “an electrode” or “the electrode” refer to a single electrode or multiple electrodes such as an electrode array. As used herein, the term “contact” as used in the context of an electrode in contact with a region of the brain refers to a physical association between the electrode and the region. An electrode can conduct electricity to specific targets in the brain. Electrodes used in the methods disclosed herein may be monopolar (cathode or anode) or bipolar (e.g., having an anode and a cathode). The electrodes may be non-brain penetrating surface electrodes, extracranial electrodes, for example, subgaleal or skull mounted (in burrhole cap or in case of cranially mounted neurostimulator) or brain-penetrating depth electrodes.

Positioning an electrode may be carried out using standard surgical procedures for placement of intra-cranial electrodes. In certain cases, placing the electrode may involve positioning the electrode on the surface of specified region(s) of the brain (e.g., Gpi and/or Gpe regions). The electrode may contact at least a portion of the surface of the brain at a specified region. In some embodiments, the electrode may contact substantially the entire surface area at the specified region. In some embodiments, the electrode may additionally contact area(s) adjacent to the specified region.

In some embodiments, an electrode array arranged on a planar support substrate may be used. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as a surface electrode) that may be used in the methods disclosed herein may be an electrocorticography (ECOG) electrode, a subgaleal electrode, or an electroencephalography (EEG) electrode. In certain embodiments, a plurality of electrodes is positioned at one or more specified brain regions (e.g., Gpi and/or Gpe regions).

In certain cases, placing the electrode at a target area or site may involve positioning a brain penetrating electrode (also referred to as depth electrode) in specified region(s) of the brain. For example, an electrode may be placed in a region of the brain (e.g., Gpi and/or Gpe). In some embodiments, the electrode may additionally contact area(s) adjacent to a specified region of the brain. In some embodiments, one or more electrodes or electrode arrays are used to target D1-MSNs and/or D2-MSNs in one or more regions of the brain (e.g., Gpi and/or Gpe).

The depth to which an electrode is inserted into the brain may be determined by the desired level of contact between the electrode array and the brain. A brain-penetrating electrode array may be obtained from a commercial supplier. A commercially obtained electrode array may be modified to achieve a desired depth of insertion into the brain tissue.

Positioning an electrode for delivering electrical stimulation to the brain may be carried out using standard surgical procedures for placement of electrodes for deep brain stimulation. For example, the electrode may be placed in a target region of the brain (e.g., Gpi and/or Gpe region). Medical imaging using, for example, magnetic resonance imaging (MRI) or computerized tomography (CT) may be used to provide guidance for placement of DBS electrodes and verify correct placement of the electrodes in the brain. In addition, a neurostimulator that generates electrical pulses is placed under the skin of the chest, typically below the collarbone or in the abdomen. In some embodiments the neurostimulator is cranially mounted. The surgical procedure may involve placing electrodes within the brain through small holes in the skull. An electrode lead is tunneled under the skin down the neck and under the skin of the chest to connect to a chest implanted neurostimulator.

Current is supplied by the neurostimulator to the electrodes. Parameters such as pulse width, shape, frequency, amplitude, pattern, and temporal distribution can be adjusted to modulate neural activity and neuronal circuits to suppress β-band oscillations. In some embodiments, a closed loop system is used to adjust DBS settings based on optimization of neurostimulation parameters and targets using the methods described herein. In other embodiments, an open loop system is used in which DBS settings are adjusted by a user or medical practitioner.

The electrical stimulation may be applied to the GPi and/or Gpe regions using a single electrode, electrode pairs, or an electrode array. In some embodiments, the number of electrodes used to deliver electrical stimulation to the brain ranges from 8 to 32, including any number of electrodes in this range such as 8, 10, 12, 14, 16, 18, 20, 22, 24, 26, 28, 30, or 32 electrodes. In some embodiments, the electrical stimulation is applied to more than one site. The site to which the electrical stimulation is applied may be alternated or otherwise spatially or temporally patterned. Electrical stimulation may be applied to the sites simultaneously or sequentially. The sites chosen for stimulation may differ for different subjects and will depend on where pathological protein aggregates are known to be present (e.g., by medical imaging) or predicted to be present.

In some embodiments, an electrode array arranged on a planar support substrate may be used for electrically stimulating a region of the brain. The surface area of the electrode array may be determined by the desired area of contact between the electrode array and the brain. In some cases, cylindrical electrode arrays, paddle-style electrode arrays, or plate-style electrode arrays may be used in the methods disclosed herein for deep brain stimulation. Such electrode arrays for implanting in the brain, may be obtained from a commercial supplier. A commercially obtained electrode/electrode array may be modified to achieve a desired contact area.

The precise number of electrodes contained in an electrode array (e.g., for electrical stimulation) may vary. In certain aspects, an electrode array may include two or more electrodes, such as 3 or more, including 4 or more, e.g., about 3 to 6 electrodes, about 6 to 12 electrodes, about 12 to 18 electrodes, about 18 to 24 electrodes, about 24 to 30 electrodes, about 30 to 48 electrodes, about 48 to 72 electrodes, about 72 to 96 electrodes, or about 96 or more electrodes. The electrodes may be arranged into a regular repeating pattern (e.g., a grid, such as a grid with about 1 cm spacing between electrodes), or no pattern. An electrode that conforms to the target site for optimal delivery of electrical stimulation may be used. One such example, is a single multi contact electrode with eight contacts separated by 21/2 mm. Each contract would have a span of approximately 2 mm. Another example is an electrode with two 1 cm contacts with a 2 mm intervening gap. Yet further, another example of an electrode that can be used in the present methods is a 2 or 3 branched electrode to cover the target site. Each one of these three-pronged electrodes has four 1-2 mm contacts with a center to center separation of 2 of 2.5 mm and a span of 1.5 mm.

The size of each electrode may also vary depending upon such factors as the number of electrodes in the array, the location of the electrodes, the material, the age of the patient, and other factors. In certain aspects, an electrode array has a size (e.g., a diameter) of about 5 mm or less, such as about 4 mm or less, including 4 mm-0.25 mm, 3 mm-0.25 mm, 2 mm-0.25 mm, 1 mm-0.25 mm, or about 3 mm, about 2 mm, about 1 mm, about 0.5 mm, or about 0.25 mm.

In certain embodiments, the method further comprises mapping the brain of the subject to optimize positioning of an electrode for applying electrical stimulation. Positioning of an electrode is optimized to maximize clinical responses to electrical stimulation to suppress β-band oscillations. In some embodiments, DBS is optimized to achieve a neurophysiologically defined change, for example, decreasing β-band synchronization and/or improving brain function.

Assessment of the effectiveness of electrical stimulation at a particular site for treating a neurological or neurodegenerative disease may be performed using any standard method. In some embodiments, the effectiveness of electrical stimulation is assessed by functional imaging the brain of the subject to determine the effectiveness of the neurostimulation in suppressing β-band oscillations. In some embodiments, the effectiveness of electrical stimulation is assessed by measuring brain function of the subject after neurostimulation. For example, brain function may be measured by performing electroencephalography (EEG), stereoelectroencephalography (sEEG), electrocorticography (ECoG), magnetoencephalography (MEG), single photon emission computed tomography (SPECT), functional magnetic resonance imaging (fMRI), optogenetic functional magnetic resonance imaging, or positron emission tomography (PET).

In some cases, the severity of symptoms of Parkinson's disease may be further assessed using a visual analog scale or a verbal rating scale. In certain embodiments, the method further comprises assessing one or more motor and/or non-motor symptoms of the subject using a Movement Disorder Society-Sponsored Revision of the Unified Parkinson's Disease Rating Scale (MDS-UPDRS), a Hoehn and Yahr (HnY) scale, or a Montreal Cognitive Assessment (MoCA) scale.

As set forth here, the subject methods involve applying electrical stimulation to the GPi and/or GPe regions. The parameters for applying the electrical stimulation to the brain may be determined empirically during treatment or may be pre-defined, such as, from a trial study with a subject. For example, varying stimulation settings may be applied including baseline (stimulation off), optimal therapeutic stimulation, modified and ineffective stimulation, and maximum tolerated stimulation to determine optimal therapeutic stimulation parameters for treatment of Parkinson's disease according to the methods described herein.

The parameters of the electrical stimulation may include one or more of frequency, pulse width/duration, duty cycle, intensity/amplitude, pulse pattern, program duration, program frequency, and the like. In certain embodiments, the parameters are adjusted to target D1-MSNs and D2-MSNs in the GPi and/or GPe regions.

Frequency refers to the pulses produced per second during stimulation and is stated in units of Hertz (Hz, e.g., 60 Hz=60 pulses per second). The frequencies of electrical stimulation used in the present methods may vary widely depending on numerous factors and may be determined empirically during treatment of the subject or may be pre-defined. In certain embodiments, the method may involve applying electrical stimulation to the brain at a frequency of 2 Hz-250 Hz, such as, 25 Hz-200 Hz, 50 Hz-250 Hz, 50 Hz-185 Hz, 50 Hz-150 Hz, 75 Hz-200 Hz, 100 Hz-200 Hz, 100 Hz-180 Hz, 100 Hz-160 Hz, or 130 Hz-150 Hz. In some embodiments, the electrical stimulation to the brain is applied at a frequency of about 120 Hz to about 160 Hz, including any pulse frequency within this range such as 120 Hz, 122 Hz, 124 Hz, 126 Hz, 128 Hz, 130 Hz, 132 Hz, 134 Hz, 136 Hz, 138 Hz, 140 Hz, 142 Hz, 144 Hz, 146 Hz, 148 Hz, 150 Hz, 152 Hz, 154 Hz, 156 Hz, 158 Hz, or 160 Hz. In some embodiments, non-integer pulse frequencies are used (e.g., 130.2 Hz, 130.4 Hz, etc.).

The electrical stimulation may be applied in pulses such as a uniphasic or a biphasic pulse. The time span of a single pulse is referred to as the pulse width or pulse duration. The pulse width used in the present methods may vary widely depending on numerous factors (e.g., severity of the disease, status of the patient, and the like) and may be determined empirically or may be pre-defined. In certain embodiments, the method may involve applying an electrical stimulation at a pulse width of about 10 μsec-500 μsec, for example, 20 μsec-450 μsec, 40 μsec-450 μsec, 60 μsec-450 μsec, 60 μsec-220 μsec, 60 μsec-120 μsec, or 60 μsec-90 μsec. In some embodiments, the electrical stimulation to the brain is applied at a pulse width of about 60 μsec to about 210 μsec, including any pulse width within this range such as 60 μsec, 65 μsec, 70 μsec, 75 μsec, 80 μsec, 85 μsec, 90 μsec, 95 μsec, 100 μsec, 105 μsec, 110 μsec, 115 μsec, 120 μsec, 125 μsec, 130 μsec, 135 μsec, 140 μsec, 145 μsec, 150 μsec, 155 μsec, 160 μsec, 165 μsec, 170 μsec, 175 μsec, 180 μsec, 185 μsec, 190 μsec, 195 μsec, 200 μsec, 205 μsec, 210 μsec, 215 μsec, or 220 μsec.

The electrical stimulation may be applied for a stimulation period of 0.1 sec-1 month, with periods of rest (i.e., no electrical stimulation) possible in between. In certain cases, the period of electrical stimulation may be 0.1 sec-1 week, 1 sec-1 day, 10 sec-12 hours, 1 min-6 hours, 10 min-1 hour, and so forth. In certain cases, the period of electrical stimulation may be 1 sec-1 min, 1 sec-30 sec, 1 sec-15 sec, 1 sec-10 sec, 1 sec-6 sec, 1 sec-3 sec, 1 sec-2 sec, or 6 sec-10 sec. The period of rest in between each stimulation period may be 60 sec or less, 30 sec or less, 20 sec or less, or 10 sec. In some embodiments, electrical stimulation may be applied for a year or more, 2 years or more, 3 years or more, 5 years or more, or 10 years or more. In some embodiments, electrical stimulation may be continued indefinitely as part of a long-term DBS therapy regimen.

The electrical stimulation may be applied with an amplitude of current of 0.1 mA-30 mA, such as, 0.1 mA-25 mA, such as, 0.1 mA-20 mA, 0.1 mA-15 mA, 0.1 mA-10 mA, 0.1 mA-2 mA, 0.1 mA-1 mA, 1 mA-20 mA, 1 mA-10 mA, 2 mA-30 mA, 2 mA-15 mA, 2 mA-10 mA, or 1 mA-3 mA. In some embodiments, the amplitude of current is 0.1 mA-3.5 mA, or any amplitude of current in this range such as 0.1 mA, 0.2 mA, 0.3 mA, 0.4 mA, 0.5 mA, 0.6 mA, 0.7 mA, 0.8 mA, 0.9 mA, 1.0 mA, 1.1 mA, 1.2 mA, 1.3 mA, 1.4 mA, 1.5 mA, 1.6 mA, 1.7 mA, 1.8 mA. 1.9 mA, 2.0 mA, 2.1 mA, 2.2 mA, 2.3 mA, 2.4 mA, 2.5 mA, 2.6 mA, 2.7 mA, 2.8 mA, 2.9 mA, 3.0 mA, 3.1 mA, 3.2 mA, 3.3 mA, 3.4 mA, or 3.5 mA.

The electrical stimulation may be applied with an amplitude of voltage of 0.1 V-15 V, such as, 0.1 V-10 V, 0.1 V-5 V, 1 V-10 V, 1 V-5, V, or 1 V-3.5 V. In some embodiments, the amplitude of voltage is 1 V-3.5 V, or any amplitude of voltage in this range such as 1 V, 1.1 V, 1.2 V, 1.3 V, 1.4 V, 1.5 V, 1.6 V, 1.7 V, 1.8 V, 1.9 V, 2.0 V, 2.1 V, 2.2 V, 2.3 V, 2.4 V, 2.5 V, 2.6 V, 2.7 V, 2.8 V, 2.9 V, 3.0 V, 3.1 V, 3.2 V, 3.3 V, 3.4 V, or 3.5 V.

The electrical stimulation having the parameters as set forth above may be applied over a program duration of around 1 day or less, such as, 18 hours, 6 hours, 3 hours, 2 hours, 1 hour, 45 minutes, 30 minutes, 20 minutes, 10 minutes, or 5 minutes, or less, e.g., 1 minute-5 minutes, 2 minutes-10 minutes, 2 minutes-20 minutes, 2 minutes-30 minutes, 5 minutes-10 minutes, 5 minutes-30 minutes, or 5 minutes-15 minutes, 10 minutes-400 minutes, 25 minutes-300 minutes, 50 minutes-200 minutes, or 75 minutes-150 minutes, which period would include the application of pulses and the intervening rest period. The program may be repeated at a desired program frequency to reduce or prevent aggregation and spreading of pathological protein aggregates and relieve symptoms of a neurological or neurodegenerative disease in the subject. As such, a treatment regimen may include a program for electrical stimulation at a desired program frequency and program duration. In some embodiments, the treatment regimen is controlled by a control unit in communication with a pulse generator connected to the one or more DBS electrodes in a closed-loop treatment regimen.

Upon completion of a treatment regimen, the patient may be assessed for effectiveness of the treatment and the treatment regimen may be repeated, if needed. In certain cases, the treatment regimen may be altered before repeating. For example, one or more of the frequency, pulse width, current amplitude, period of electrical stimulation, program duration, program frequency, and/or placement of DBS or detection electrodes may be altered before starting a second treatment regimen.

Application of the method may include a prior step of selecting a patient for treatment based on need as determined by clinical assessment, which may include assessment of severity of a neurological or neurodegenerative disease (e.g., a neurological or neurodegenerative disease lasting at least 3 months), physical condition, medication regime, cognitive assessment, anatomical assessment, behavioral assessment and/or neurophysiological assessment. In certain cases, a subject may be further assessed to determine if neurostimulation will completely or partially (e.g., at least 50%) relieve the neurological or neurodegenerative disease. Such a patient may undergo neurostimulation on a temporary trial basis to determine if neurostimulation suppresses β-band oscillations or decreases the severity of symptoms experienced by the patient.

### Kits

Also provided are kits comprising software for carrying out the computer implemented methods, described herein, for modeling propagation of β-band oscillations in the brain of a subject and the response to neuromodulation. In some embodiments, the kit comprises a non-transitory computer-readable medium and instructions for optimizing neurostimulation parameters and targets based on modeling propagation of β-band oscillations in the brain of a subject and the response to neuromodulation using the computer implemented methods, as described herein. In some embodiments, the kit comprises a system comprising a processor programmed according to a computer implemented method described herein; and a display component for displaying information regarding the propagation of β-band oscillations in the brain of the subject and the response to neuromodulation.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-53 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method of suppressing β-band oscillations in the brain of a subject by performing optogenetic neuromodulation of medium spiny neurons according to a method comprising:
(a) optogenetically inhibiting D1-medium spiny neurons (D1-MSNs) in a globus pallidus internal (GPi) region of the brain of the subject, wherein D1-MSN mediated β-band oscillations are suppressed;
(b) optogenetically inhibiting D2-medium spiny neurons (D2-MSNs) in a globus pallidus external (Gpe) region of the brain of the subject, wherein D2-MSN mediated β-band oscillations are suppressed;
(c) optogenetically stimulating the D1-MSNs periodically in the Gpe region of the brain of the subject, wherein D1-MSN mediated β-band oscillations are suppressed; or
(d) optogenetically stimulating the D1-MSNs and the D2-MSNs randomly in the Gpe region of the brain of the subject, wherein D1-MSN mediated β-band oscillations and D2-MSN mediated β-band oscillations are suppressed; or any combination of (a)-(d).
2. The method of aspect 1, wherein the method comprises:
(a) optogenetically inhibiting the D1-MSNs in the GPi region of the brain of the subject, wherein the D1-MSN mediated β-band oscillations are suppressed;
(b) optogenetically inhibiting the D2-MSNs in the Gpe region of the brain of the subject, wherein the D2-MSN mediated β-band oscillations are suppressed;
(c) optogenetically stimulating the D1-MSNs periodically in the Gpe region of the brain of the subject, wherein the D1-MSN mediated β-band oscillations are suppressed; and
(d) optogenetically stimulating the D1-MSNs and the D2-MSNs randomly in the Gpe region of the brain of the subject, wherein the D1-MSN mediated β-band oscillations and the D2-MSN mediated β-band oscillations are suppressed.
3. The method of aspect 1 or 2, wherein said optogenetically inhibiting the D1-MSNs or the D2-MSNs in the Gpi region or the Gpe region comprises:
introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and
illuminating the light-responsive ion channel with light at a wavelength that activates the light-responsive ion channel, wherein conduction of ions by the light-responsive ion channel in response to absorption of light results in hyperpolarization and inhibition of the D1-MSNs or the D2-MSNs.
4. The method of aspect 3, wherein the light-responsive ion channel is a light-responsive anion-conducting opsin or a light-responsive proton conductance regulator.
5. The method of aspect 4, wherein the light-responsive anion-conducting opsin conducts chloride ions (Cl⁻).
6. The method of aspect 4 or 5, wherein the anion-conducting opsin is an anion-conducting channelrhodopsin or halorhodopsin.
7. The method of aspect 6, wherein the halorhodopsin is a Natronomonas pharaonis halorhodopsin (NpHR), enhanced NpHR (eNpHR) 1.0, eNpHR 2.0, or eNpHR 3.0.
8. The method of aspect 6, wherein the anion-conducting channelrhodopsin is iC1C2, SwiChR, SwiChR++, or iC++.
9. The method of aspect 4, wherein the light-responsive proton conductance regulator is a bacteriorhodopsin or an archaerhodopsin.
10. The method of aspect 9, wherein the light-responsive proton conductance regulator is Arch from Halorubrum sodomense, ArchT from Halorubrum sp., TP009 from Leptosphaeria maculans, or Mac from Leptosphaeria maculans.
11. The method of aspect 1 or 2, wherein said optogenetically stimulating the D1-MSNs or the D2-MSNs in the Gpi region or the Gpe region comprises:
introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and
illuminating the light-responsive ion channel with light at a wavelength that activates the light-responsive ion channel, wherein conduction of ions by the light-responsive ion channel in response to absorption of light results in depolarization and activation of the D1-MSNs or the D2-MSNs.
12. The method of aspect 11, wherein the light-responsive ion channel is a light-responsive cation-conducting opsin.
13. The method of aspect 12, wherein the light-responsive cation-conducting opsin conducts calcium cations (Ca²⁺).
14. The method of aspect 12 or 13, wherein the light-responsive cation-conducting opsin is a light-responsive cation-conducting channelrhodopsin.
15. The method of aspect 14, wherein the light-responsive cation-conducting channelrhodopsin is a Chlamydomonas reinhardtii channelrhodopsin or a Volvox carteri channelrhodopsin.
16. The method of aspect 15, wherein the light-responsive cation-conducting channelrhodopsin is a Chlamydomonas reinhardtii channelrhodopsin-1 (ChR1), a Chlamydomonas reinhardtii channelrhodopsin-2 (ChR2), a Volvox carteri channelrhodopsin-1 (VChR1), or a chimeric ChR1-VChR1 channelrhodopsin.
17. The method of any one of aspects 1-16, wherein the polynucleotide encoding the light-responsive ion channel is provided by a viral vector.
18. The method of aspect 17, wherein the viral vector is a lentiviral vector or an adeno-associated viral (AAV) vector.
19. The method of aspect 17 or 18, wherein the viral vector is stereotactically injected into the retrosplenial cortex.
20. The method of any one of aspects 17-19, wherein the vector further comprises a neuron-specific promoter operably linked to the polynucleotide encoding the light-responsive ion channel.
21. The method of any one of aspects 17-20, wherein expression of the light-responsive ion channel is inducible.
22. The method of any one of aspects 1-21, wherein said illuminating the light-responsive ion channel comprises delivering light from a light source to the light-responsive ion channel using a fiber-optic-based optical neural interface.
23. The method of aspect 22, wherein the light source is a solid-state diode laser.
24. The method of any one of aspects 1-23, wherein the subject has Parkinson's disease.
25 The method of any one of aspects 1-24, wherein said optogenetically inhibiting the D1-MSNs comprises sustained shunting inhibition of the D1-MSNs in the GPi region of the brain of the subject.
26. The method of any one of aspects 1-25, wherein said optogenetically inhibiting the D2-MSNs comprises sustained shunting inhibition of the D2-MSNs in the GPe region of the brain of the subject.
27. The method of any one of aspects 1-26, wherein said optogenetically stimulating the D1-MSNs periodically in the Gpe region of the brain of the subject comprises performing periodic stimulation at a frequency of 130 Hz.
28. The method of any one of aspects 1-27, wherein said optogenetically stimulating the D1-MSNs and the D2-MSNs randomly in the Gpe region of the brain of the subject comprises using a plurality of stimulation sequences with randomly spaced pulses of 130 Hz, wherein each neuron is stimulated with one of the stimulation sequences such that synchronized neurons are decoupled.
29. The method of any one of aspects 1-28, wherein said optogenetically stimulating the D1-MSNs or the D2-MSNs comprises direct activation of the D1-MSNs or the D2-MSNs with a strength of 500 pA, 700 pA, or 900 pA.
30. A method of treating Parkinson's disease in a subject, the method comprising:
positioning a first electrode at a first location in a globus pallidus internal (GPi) region of the brain of the subject to deliver electrical stimulation to D1-medium spiny neurons in the Gpi region;
positioning a second electrode at a second location in a globus pallidus external (Gpe) region of the brain of the subject to deliver electrical stimulation to D1-medium spiny neurons and D2-medium spiny neurons in the Gpe region; and
applying electrical stimulation to the Gpi region of the brain of the subject using the first electrode and applying electrical stimulation to the Gpe region of the brain of the subject using the second electrode in a manner effective to suppress β-band oscillations to treat Parkinson's disease.
31. The method of aspect 30, wherein the electrical stimulation is applied with the first electrode or the second electrode unilaterally or bilaterally.
32. The method of aspect 30 or 31, wherein the first electrode or the second electrode is a depth electrode or a surface electrode.
33. The method of any one of aspects 30-32, wherein the first electrode or the second electrode is a non-brain penetrating surface electrode array or a brain-penetrating electrode array.
34. The method of any one of aspects 30-33, wherein the first electrode is placed on a surface of the Gpi region.
35. The method of any one of aspects 30-34, wherein the second electrode is placed on a surface of the Gpe region.
36. The method of any one of aspects 30-35, wherein the method further comprises assessing effectiveness of the treatment in the subject using a visual analog scale, a verbal rating scale, a Movement Disorder Society-Sponsored Revision of the Unified Parkinson's Disease Rating Scale (MDS-UPDRS), a Hoehn and Yahr (HnY) scale, or a Montreal Cognitive Assessment (MoCA) scale.
37. A computer implemented method for modeling propagation of β-band oscillations in a brain of a subject and response to neuromodulation, the computer performing steps comprising:
a) receiving functional magnetic resonance imaging data of neural activity before optogenetic stimulation and during optogenetic stimulation of D1-medium spiny neurons (D1-MSNs) and D2-medium spiny neurons (D2-MSNs) in a caudate putamen (CPu) region, an external globus pallidus (GPe) region, an internal globus pallidus (GPi) region, a subthalamic nucleus (STN) region, a substantia nigra pars reticulata (SNr) region, a thalamus (THL) region, and a motor cortex (MCX) region of the brain of the subject;
b) performing spectral dynamic causal modeling of effective connectivity strengths among the CPu region, the GPe region, the GPi region, the STN region, the SNr region, the THL region, and the MCX region;
c) receiving experimental electrophysiological data for the CPu region;
d) estimating effective connectivity strengths of GABAergic connections using the experimental electrophysiological data for the CPu region;
e) estimating effective connectivity strengths of glutamatergic connections using dynamic causal modeling of the functional magnetic resonance imaging data;
f) performing biophysics modeling using a Hodgkin-Huxley model to generate simulated electrophysiology data using the effective connectivity strength estimates;
g) optimizing GABAergic projections iteratively until the simulated electrophysiology data matches the experimental electrophysiological data for the CPu region;
h) calculating a temporal profile of average power of beta-band frequencies for each neuron in the CPu region, the GPe region, the GPi region, the STN region, the SNr region, the THL region, and the MCX region; and
i) comparing total amount of co-occurred beta-band oscillation power before optogenetic stimulation to co-occurred beta-band oscillation power during optogenetic stimulation to model the propagation of β-band oscillations in the brain of the subject.
38. The computer implemented method of aspect 37, wherein the D1-MSN are Huxley-Hudgkin neurons.
39. The computer implemented method of aspect 37 or 38, wherein the experimental electrophysiology data comprise single-neuron recordings.
40. The computer implemented method of aspect 39, wherein the single-neuron recordings are from GABAergic neurons of the CPu region.
41. The computer implemented method of any one of aspects 37-40, wherein glutamatergic connections are modeled as 1-to-1 connections with connection strengths proportional to effective connectivity estimated by the DCM.
42. The computer implemented method of any one of aspects 39-41, wherein CPu-GPi/GPe and GPi/SNr-thalamus GABAergic projections are modeled as 1-to-n diffusive projections with connectivity strength wGABA, where n and wGABA are free parameters, wherein N and wGABA are searched across parameter space until the simulated spike rates match statistically with the single-neuron recordings.
43. The computer implemented method of any one of aspects 37-42, wherein the optogenetic stimulation comprises optogenetically inhibiting the D1-MSNs in the GPi region of the brain of the subject.
44. The computer implemented method of aspect 43, wherein said optogenetically inhibiting the D1-MSNs comprises sustained shunting inhibition of the D1-MSNs in the GPi region of the brain of the subject.
45. The computer implemented method of any one of aspects 37-44, wherein the optogenetic stimulation comprises optogenetically inhibiting the D2-MSNs in the GPe region of the brain of the subject.
46. The computer implemented method of aspect 45, wherein said optogenetically inhibiting the D2-MSNs comprises sustained shunting inhibition of the D2-MSNs in the GPe region of the brain of the subject.
47. The method of any one of aspects 37-46, wherein the optogenetic stimulation comprises direct activation of the D1-MSNs or the D2-MSNs with a strength of 500 pA, 700 pA, or 900 pA.
48. The computer implemented method of any one of aspects 37-47, wherein the optogenetic stimulation comprises optogenetically stimulating the D1-MSNs periodically in the Gpe region of the brain of the subject.
49. The computer implemented method of any one of aspects 37-48, wherein the optogenetic stimulation comprises optogenetically stimulating the D1-MSNs and the D2-MSNs randomly in the Gpe region of the brain of the subject using a plurality of stimulation sequences with randomly spaced pulses, wherein each neuron is stimulated with one of the stimulation sequences such that synchronized neurons are decoupled.
50. The computer implemented method of any one of aspects 37-49, wherein the optogenetic stimulation is performed with periodic stimulation at a frequency of 130 Hz.
51. A system for modeling propagation of β-band oscillations in a brain of a subject using the computer implemented method of any one of aspects 37-50, the system comprising:
a) a storage component for storing data, wherein the storage component has instructions for modeling propagation of β-band oscillations based on analysis of the functional magnetic resonance imaging data and the experimental electrophysiology data stored therein;
b) a computer processor for processing the functional magnetic resonance imaging data and the experimental electrophysiology data using one or more algorithms, wherein the computer processor is coupled to the storage component and configured to execute the instructions stored in the storage component in order to receive the inputted functional magnetic resonance imaging data and the experimental electrophysiology data and analyze the data according to the computer implemented method of any one of aspects 37-50; and
c) a display component for displaying the information regarding the propagation of β-band oscillations in the brain of the subject.
52. A non-transitory computer-readable medium comprising program instructions that, when executed by a processor in a computer, causes the processor to perform the method of any one of aspects 37-50.
53. A kit comprising the non-transitory computer-readable medium of aspect 52 and instructions for modeling the propagation of β-band oscillations from the functional magnetic resonance imaging data and the experimental electrophysiology data.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventors to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Optogenetically-Driven β-Synchrony Reveals Non-Canonical Dynamics Along the Basal Ganglia Direct and Indirect Pathways

Neuronal firing rates and patterns are both crucial in untangling the dynamics of brain functions and dysfunctions¹⁻³. For example, in Parkinson's disease (PD), altered firing rates and patterns, in the form of abnormal β-band (12-30 Hz) oscillations have been observed across the cortico-basal-ganglia-thalamus (CBT) network⁴⁻⁶. Understanding how the CBT network mediate neural oscillations and how the neural oscillations are related to the firing rates of individual cell populations can facilitate decoding of the circuit mechanism underlying PD pathology and improve neuromodulation treatments.

The pathological changes in firing rates with PD are conventionally explained by the canonical model of the basal ganglia circuitry^{4,5}. The canonical model attributes motor facilitation and suppression to the assumed direct and indirect pathways, mediated by D1- and D2-receptor expressing medium spiny neurons (MSNs) in striatum⁷. In PD, the D2-MSNs are assumed to be hyperactive, causing the persistent activation of the indirect pathway⁴. Despite the wide applications of this model in explaining experimental results and disorders involving basal ganglia, the model cannot explain many recent experimental findings. For example, the globus pallidus internal (GPi) and thalamus are found to be activated concurrently during voluntary actions and stimulations, whereas the canonical model proposes that the activity of the two regions have opposite polarities⁸⁻¹¹.

Excessive oscillations in β-band is another hallmark of PD^{6,12,13}, whose mechanisms have not been explained by the spike-rate based canonical basal ganglia model. In healthy conditions, β-band oscillations in the CBT circuit is commonly observed and assumed to play a role in maintaining status quo¹⁴⁻¹⁷. Excessive β-band oscillations in the CBT circuit in PD may reflect overstabilization, causing abnormal persistence of current motor state (Bradykinesia)¹⁶. Deep brain stimulation (DBS) can alleviate the pathological synchronization in STN, GPi^{18,19}, and cortical regions²⁰. Since indirect pathway is assumed to be involved in anti-kinetic control and indirect pathway nodes have been shown to be hyperactive in PD^{4,5}, β-band oscillations have been assumed to propagate along the indirect pathway^{21,22}. However, the assumption has not been examined at the scale of whole CBT network and the specific mechanisms of how basal ganglia circuitries contribute to the propagation of β-band synchronization remains elusive. In this study, we utilized optogenetically induced synchrony, optogenetically amplified spontaneous activity, and computational modeling to dissect the contributions of basal ganglia pathways to the propagation of β-band oscillations. We show that the mechanisms underlying β-band oscillation can also explain recent firing-rate based observations that cannot be explained by the canonical model.

### Optogenetic Stimulation and In Vivo Electrophysiology Suggest the Direct Pathway can Mediate β-Band Oscillation.

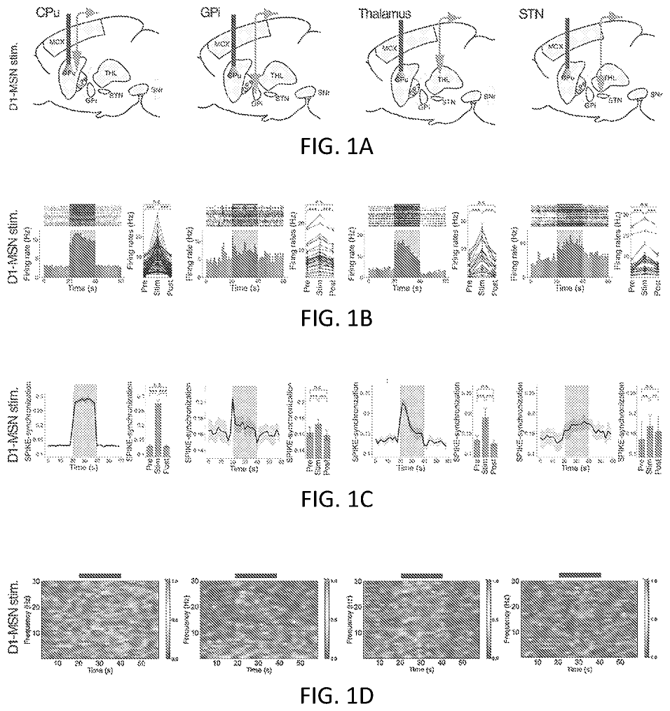

To investigate how CBT network mediates β-band oscillations and understand the previous findings that cannot be explained by the canonical model, we utilized optogenetics and in vivo extracellular recordings. To investigate the direct pathway contribution to β-band oscillations, D1-MSNs in striatum were optogenetically stimulated at β-band (20 Hz) (see “Methods”). Extracellular recordings were collected from 4 ROIs, including caudate putamen (CPu), GPi, thalamus, and subthalamic nucleus (STN), during D1-MSN β-band stimulation (FIGS. 1A and 1B). Electrophysiology recordings in thalamus, targeted at the ventrolateral nucleus, show increased firings during D1-MSN stimulation, which agree with the canonical direct pathway model. However, GPi and STN both exhibit increased activity that are at odds with the canonical model.

We then employed SPIKE-synchronization and power spectral density (PSD) profiles to measure the synchronization variations among the neurons identified in extracellular recordings within each ROI. SPIKE-synchronization is a time-resolved synchronization measurement based on the relative number of coincidental appearances of spikes²³. D1-MSN optogenetic stimulations successfully induce β-band synchrony in CPu (FIGS. 1C and 1D), GPi, thalamus with PSD increase around 20 Hz (FIG. 1D and FIG. S5D). STN neurons show increased synchrony without any beta-band peak (FIG. 1D). When neuronal spikes of each ROI were aligned with laser pulse cycles (8 ms on in every 50 ms). We find that the neurons of CPu, GPi, and thalamus fire with interlocked phases at 20 Hz (FIG. 1E). Phase-locking oscillations involving GABAergic connections have been shown to result in post-inhibition rebound firings^{8,10}. Therefore, we hypothesize that the excitation in GPi and thalamus are likely driven by post-inhibition rebound firings. These results together show that the β-oscillation can be propagated via the direct pathway in a phase-locking manner.

### Optogenetic Simulation and In Vivo Electrophysiology Suggest the Indirect Pathway Partially Mediates β-Band Oscillation.

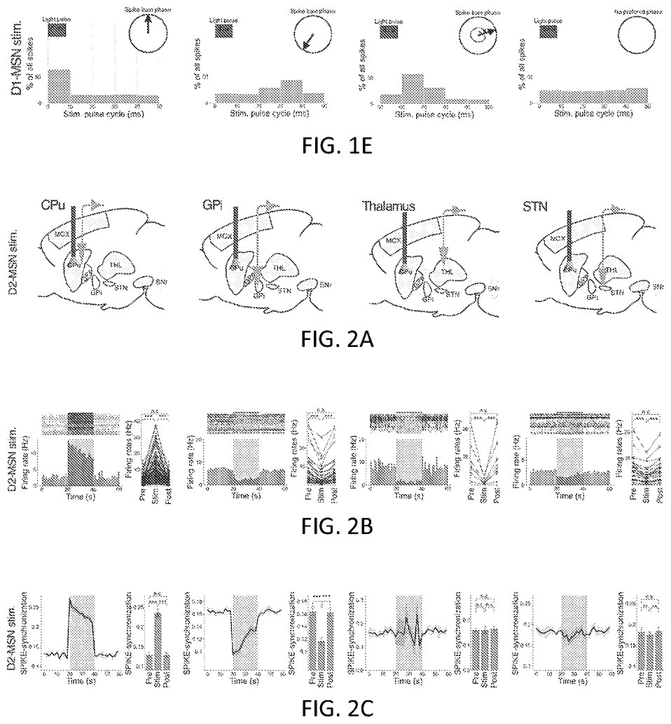

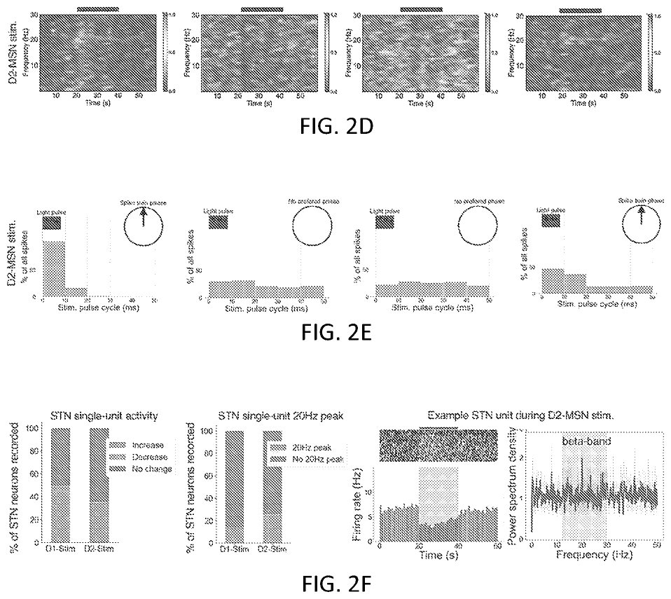

To investigate the direct pathway contribution to β-band oscillations, D2-MSNs in striatum were optogenetically stimulated at β-band (20 Hz) (see “Methods”) and in vivo extracellular recordings were collected from CPu, GPi, thalamus, and STN (FIGS. 2A and 2B). The increase of CPu firing rate during D2-MSN stimulation confirms the successful activation of D2-MSNs. The firing rate of thalamus decreased during D2-MSN stimulation, which agrees with the prediction of canonical indirect pathway model. However, the decreased firings in GPi and STN cannot be explained with the canonical model.

Decreased synchronization levels are found in GPi and STN during D2-MSN stimulation (FIG. 2C). The PSD of CPu and STN peak at 20 Hz during stimulation, which confirms the successful D2-MSN stimulation, whereas the 20 Hz peak doesn't exist in thalamus (FIG. 2D). The synchronized subgroup of STN neurons exhibit in-phase firings with CPu (FIG. 2E). The PSDs of 22/90 (24.44%) STN neurons exhibit highest power at 20 Hz (FIG. 2F). However, the synchronization in STN does not propagate to GPi and thalamus (FIGS. 2C and 2D).

### Modeling D1- and D2-MSN Stimulation Networks Reveals the Mechanism of β-Oscillation Propagation.

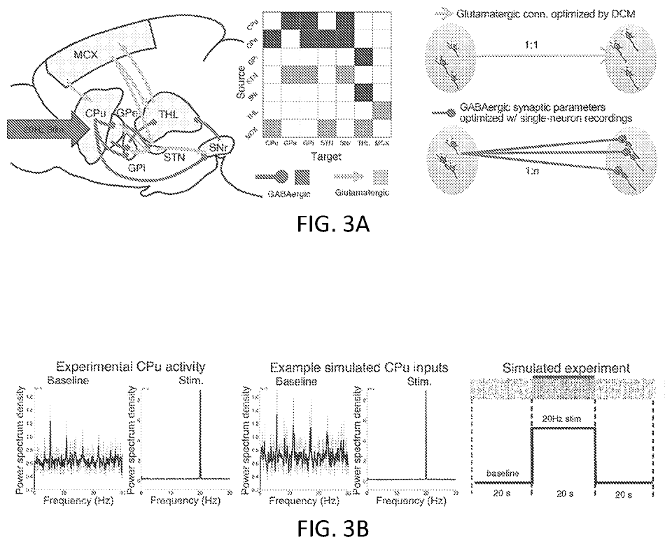

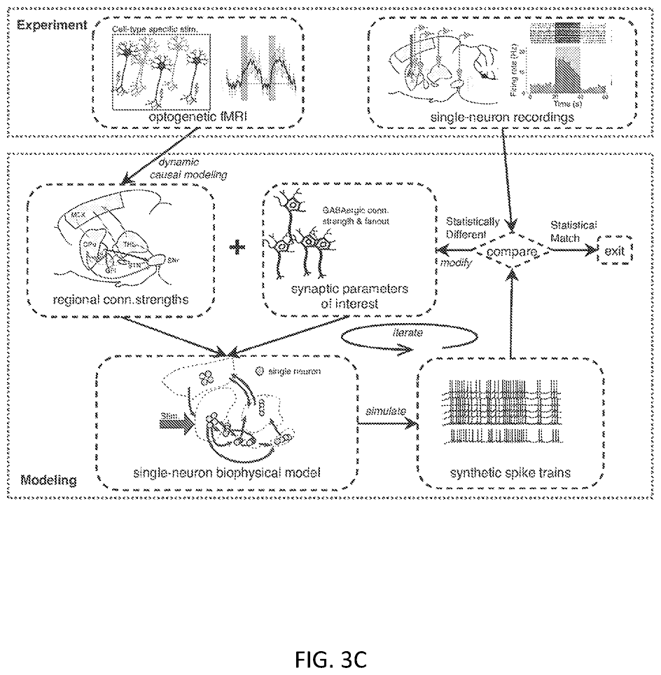

To understand the novel dynamics and how they are related to the β-band oscillation propagation, we then built a large-scale neural network model that can directly simulate D1- and D2-MSN β-band stimulations at the single-cell spiking level. We constructed 7 modules to represent the 7 ROIs in the CBT network (FIG. 3A). Considering the distinct biophysical properties between the GABAergic projections and the glutamatergic projections, the two types of connections were modeled separately (FIG. 3A, see Methods for more details). Model inputs were sampled spike trains from CPu extracellular recordings (FIG. 3B). We then developed a model following the experimental design and optimization procedure shown in FIG. 3C. For glutamatergic projection strength parameter, we utilized regional connectivity estimates from dynamic causal modeling^{9,24} of optogenetic fMRI BOLD time series (FIG. S2, S3, Table S1-S6, see “Methods” for more details). Parameters for the GABAergic projections were optimized iteratively until the simulated electrophysiology data matched experimental results (FIG. 3c, also see “Methods”).

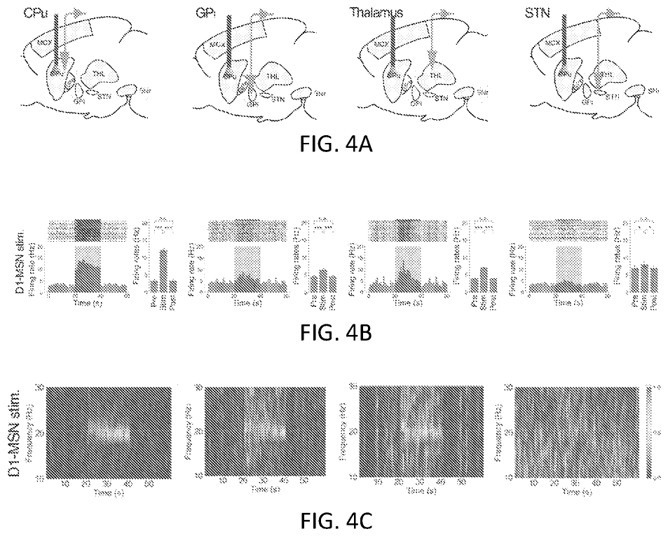

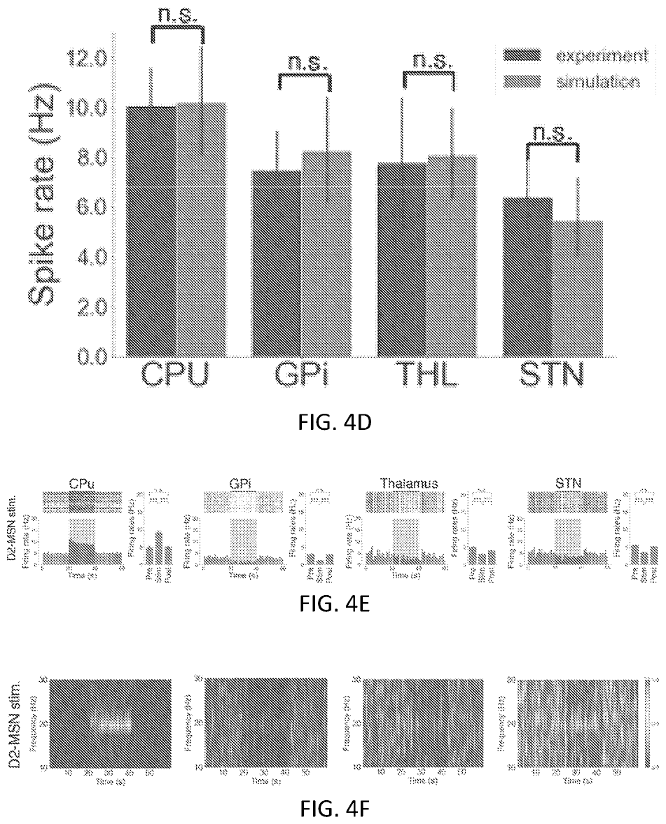

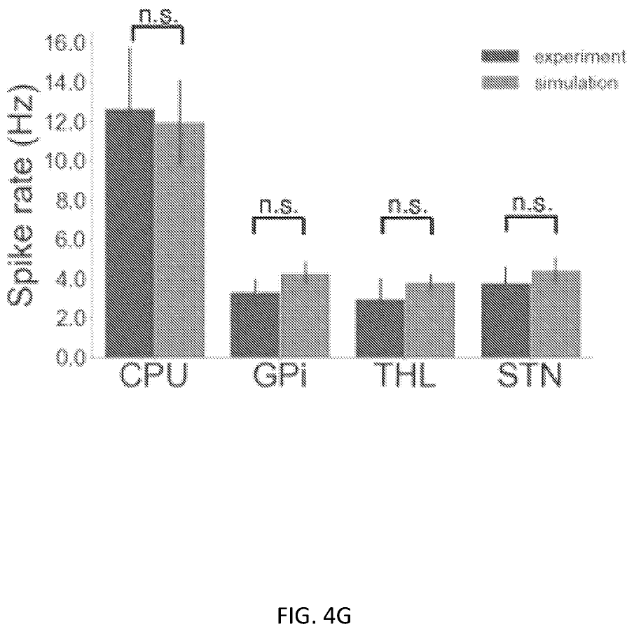

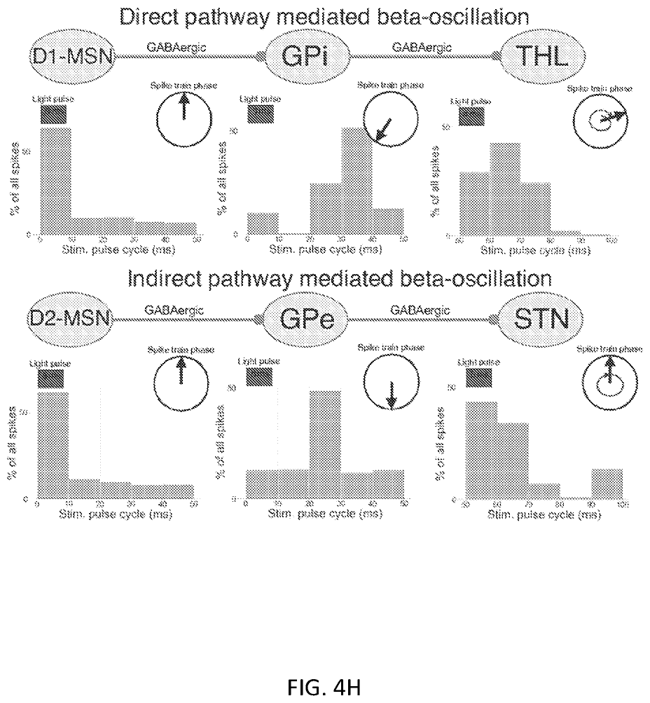

The simulated spike trains successfully mimic the firing and spectral patterns we measured in CPu, GPi, thalamus, and STN (FIGS. 4B-4G and FIG. S5). Simulated D1- and D2-MSN stimulation enabled us to fully investigate individual neuronal spiking activity and the group dynamics across the ROIs. Based on the simulations, we could clearly observe phase locking (FIG. 4H) during stimulation of D1-MSN when GPi and thalamus are excited (FIG. 4B). This suggests that the mechanisms underlying the unexpected excitations observed in GPi and thalamus is post-inhibition rebound firing. The firing rates of GPi, thalamus, and STN decreased during D2-MSN stimulation (FIG. 4E), which match our observations from electrophysiology recordings (FIG. 4G). Simulated D2-MSN stimulation synchronized STN via phase-locking rebound firings along the indirect pathways (FIG. 4H), but not GPi and thalamus (FIG. 4F). In both measured data and simulations, during D1-MSN stimulation, GPi and thalamus exhibited β-band peak, and increased firing rates. During D2-MSN stimulation, GPe exhibited β-band peak and increased firing rates, but STN exhibited β-band peak (FIGS. 2E and 4F) while being hypoactive (FIGS. 2B and 4E). To understand this phenomenon, we searched the parameter space of projection strength and axonal density in target in our simulations. This revealed that GPe-STN projection may have higher axonal branching than other β-oscillation mediating connections, which is consistent with anatomical studies 25 (FIG. 4I).

### Optogenetically Enhanced Spontaneous Activities Validate β-Oscillation Propagation Pathways.

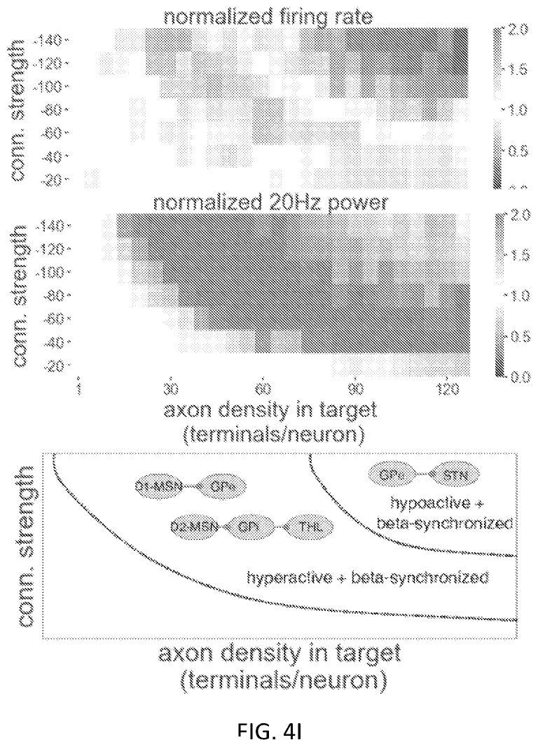

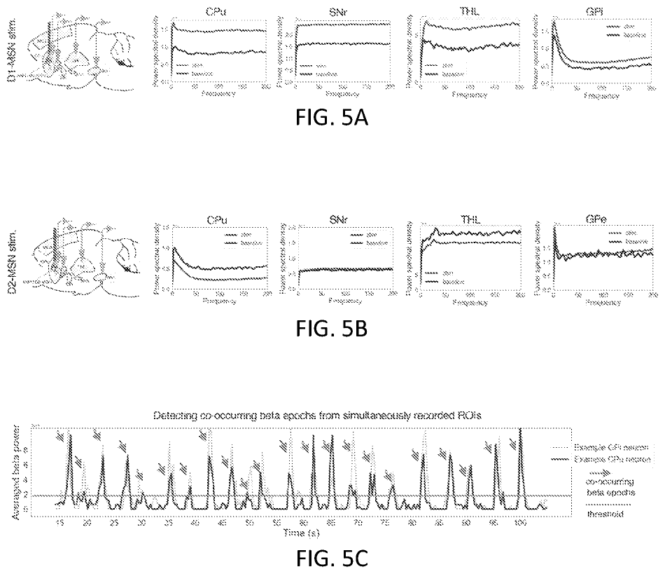

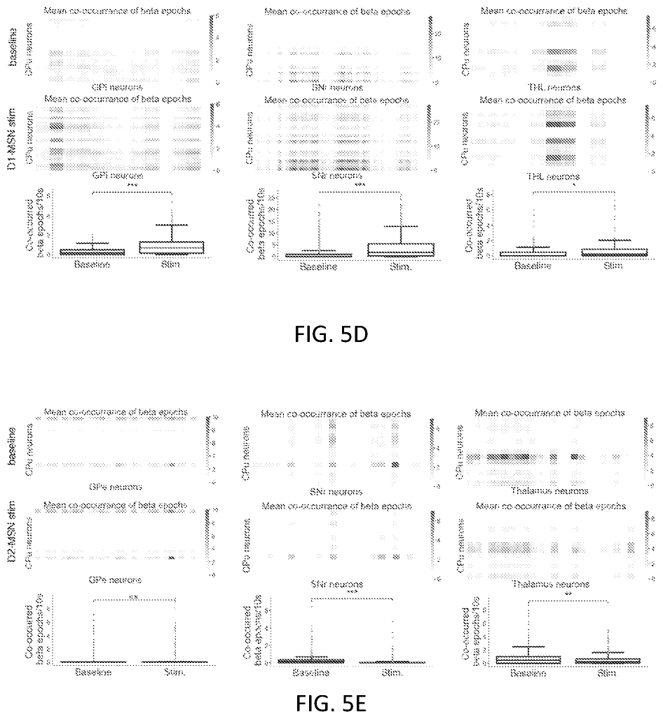

To further investigate whether the propagation pathways of β-band oscillations revealed with optogenetically induced synchrony is generalizable to β-band oscillations during spontaneous activities, we utilized stabilized step-function opsin (SSFO) to selectively enhance the spontaneous activity of D1- and D2-MSNs, respectively. Activating D1-MSNs led to broad-band increase in CPu activities without changing the general power spectral distribution, which confirms that SSFO amplifies the endogenous activity without inducing synchronization (FIG. 5A). D1-MSN activation also increased thalamic activity in a broad-band manner, which proves the enhanced spontaneous activity of the direct pathway. Activating D2-MSN led to broad-band decrease in CPu activity, which may be due to lateral inhibition, and the thalamic broad-band activity decrease confirms the enhanced spontaneous activity of the indirect pathway (FIG. 5B).

Simultaneous extracellular recordings were conducted for CPu-GPi, CPu-SNr, and CPu-thalamus pairs during D1-MSN activations. Likewise, CPu-GPe, CPu-SNr, and CPu-thalamus simultaneous extracellular recordings were conducted during D2-MSN activations. With these simultaneously recorded single-neuron spike trains, we detected the co-occurrence of β-oscillation epochs (FIG. 5C) for all pairs of neurons. Enhanced D1-MSN spontaneous activity significantly increased the amount of co-occurring β-oscillation for CPu-GPi, CPu-SNr, and CPu-thalamus pairs (FIG. 5D). The increased β-oscillation co-occurrence in CPu-GPi and CPu-SNr pairs during D1-MSN activation cannot be explained by the canonical model, which expects D1-MSN activation to deactivate GPi and SNr. On the other hand, these results are consistent with our synchronous ChR2 D1-MSN stimulation experiment results and biophysical modeling, which showed that D1-MSN can excite GPi and SNr. This provides strong evidence that direct pathway can mediate β-oscillations. Spontaneous SSFO D2-MSN activations caused decrease in co-occurring β-oscillation epochs for CPu-SNr and CPu-thalamus pairs (FIG. 5E), which confirms that indirect pathway mediated β-oscillations cannot propagate to SNr and thalamus.

### Simulated Neuromodulation Experiments Successfully Predict Optimal Neuromodulation Targets for the Interruption of β-Band Oscillations.

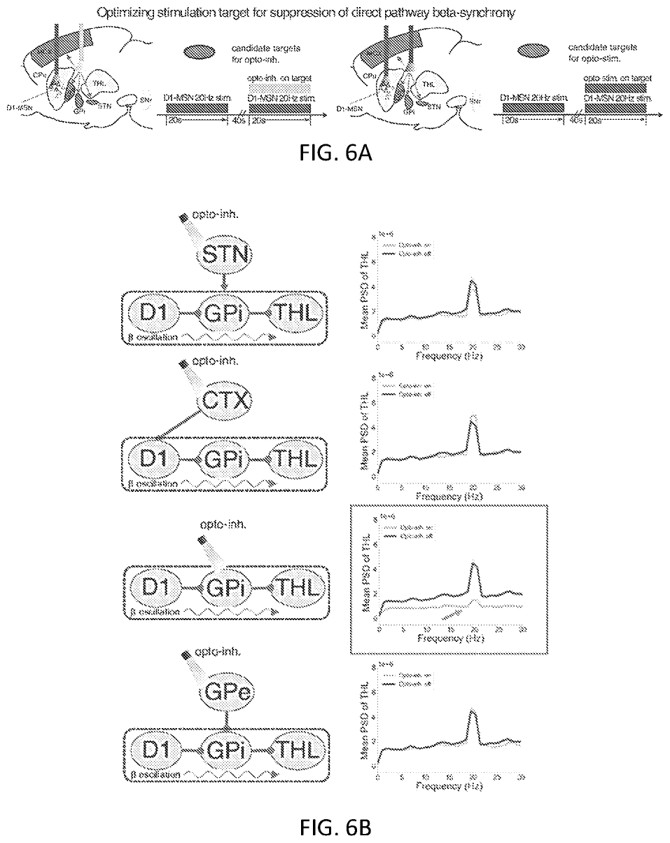

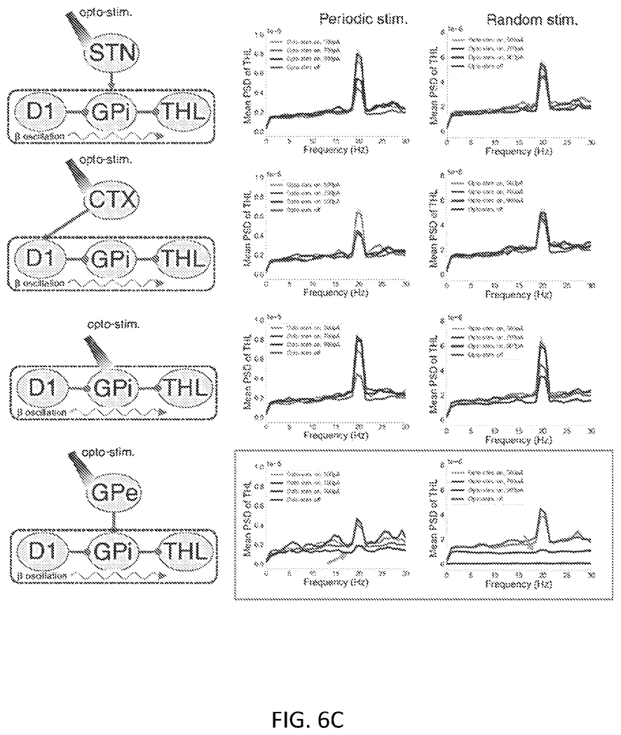

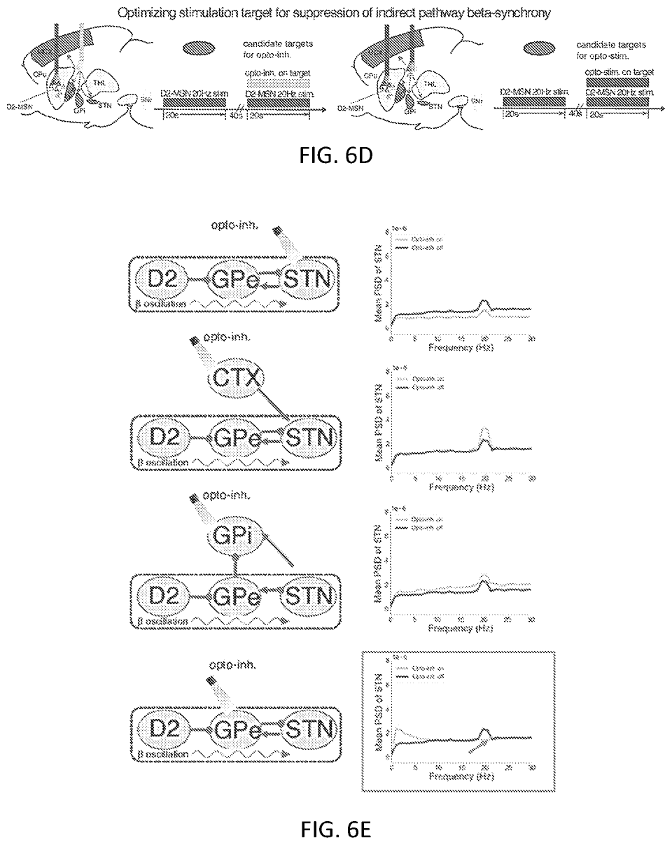

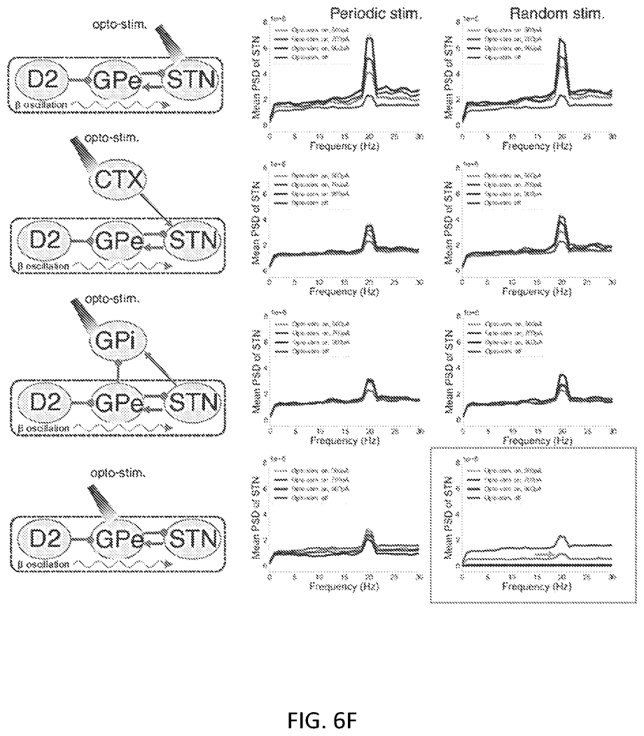

We next sought to test whether our model can predict optimal neuromodulation targets and parameters for interrupting the β-band synchronizations. We induced β-band oscillations along the direct and indirect pathways via D1- and D2-MSN stimulations, respectively. GPi, GPe, STN, and motor cortex were chosen as candidate neuromodulation targets. We simulated optogenetic inhibitions, optogenetic periodic stimulations, and optogenetic random stimulations on each target (FIGS. 6A and 6D). The effects were evaluated based on the thalamus PSD profiles for D1-MSN mediated β-band oscillations, and the STN PSD profiles for D2-MSN mediated β-band oscillations. With opto-inhibition, GPi is the only target that can eliminate the β-band oscillations along the direct pathway (FIG. 6B), and GPe is the only target that can eliminate the β-band oscillations along the indirect pathway (FIG. 6E). Interestingly, the results match results from a opto-inhibition study with a 6-OHDA rat PD model 26, which reported that the inhibition of globus pallidus, not STN and cortex, was able to remove the abnormal synchronizations in the CBT network. With periodic opto-stimulations, GPe is the only effective target that can interrupt β-band oscillations along the direct pathways with the highest stimulation strength (FIG. 6C). With random opto-stimulations, GPe stimulations reduced the β-band oscillations along both the direct and indirect pathways, but the interruptions are broadband (FIGS. 6C and 6F). Complete simulation results are shown in FIG. S6-S8. These results suggest the central role of GPi and GPe in orchestrating excessive β-band synchronization in the CBT network.

### Discussion

Through optogenetically driven synchronization and optogenetically modulated spontaneous activity experiments, we show that β-band oscillations can be propagated via the direct pathway and partially via the indirect pathway. Traditionally, β-band oscillations were assumed to propagate along the D2-MSN mediated indirect pathway. This is because: 1) in both healthy and PD conditions, β-band oscillations are reportedly anti-kinetic and the indirect pathway is assumed to be anti-kinetic^{4,5,14}; 2) in PD, the indirect pathway (D2-MSN→GPe→STN) is found to be hyperactive and β-band oscillations have been observed in GPe and STN^{6,27}. However, this assumption has not been verified directly on the scale of whole CBT circuitry and recent experimental findings related to the propagation of β-band oscillations are at odds with the canonical direct/indirect model⁹. We utilized optogenetic manipulations and single-unit recordings covering multiple key nodes along the direct and indirect pathways. With these data, we built a large-scale, spiking-level model. We showed that this model's capability to uncover the mechanisms underlying β-band oscillations, and to optimize neuromodulation targets and parameters to suppress β-band oscillations.

The GPi/SNr-thalamus “paradoxical” co-activation reported by many studies is one major counter evidence against the canonical firing-rate model of basal ganglia⁸⁻¹¹. For example, a recent study using 6-OHDA rats observed phase-locking β-band oscillations in SNr and the motor thalamus, which disagrees with the thalamic hypoactivity prediction by canonical models of PD¹¹. These results can be perfectly explained by our experiments and modeling, which showed that β-band oscillations mediated by D1-MSN leads to co-activation of GPi/SNr and thalamus in a phase-locking manner. Our computational modeling explains that the GPi/SNr-thalamus co-activation is due to post-inhibition rebound firings. These findings are supported by Kim et al.¹⁰, in which they show GPi-thalamus coactivation is dependent on post-inhibition rebound firings.

The anti-kinetic nature of β-band oscillations does not eliminate the possibility of the direct pathway mediating β-band oscillations. Activation of D1-MSN, or the direct pathway, is not always pro-kinetic. Yttri et al.²⁸ reported that both the direct and indirect pathways are capable of modulating movements bi-directionally, in which they showed the optogenetic stimulations (15 Hz) of D1-MSN reduced the velocity of fast movements. Studies also have argued that D1- and D2-MSN work more cooperatively rather than playing separated roles²⁹. A recent study showed that under certain conditions, β-band oscillations increase with striatal dopamine level increase, which favors D1-MSN activation and D2-MSN suppression.

The origin of excessive β-band oscillations in PD and whether it involves the same circuitry as in healthy conditions are still debated^{21,22,31}. The synchrony along the indirect pathway that we observed could be amplified via synaptic changes between GPe and STN following PD onset²⁷. Though PD pathological changes may preferably activate the indirect pathway, we showed that increase in excitability of D2-MSN alone cannot induce excessive β-band oscillations (FIG. 5B). D1-MSN hypoactivity in PD does not necessarily limit the possibility of β-band oscillations propagating via the direct pathway. We have shown that STN mediates β-band oscillations while being hypoactive (FIG. 2F), and D1-MSN may mediate β-band oscillations in PD through similar mechanisms. Using 6-OHDA mice, Zemel et al.³² found increased β-band oscillations in striatum only during contralateral rotations although the mice spent majority of the time in ipsilateral rotations. As shown in several previous experimental studies^{7,9}, contralateral rotations correspond to D1-MSN dominant activity while ipsilateral rotations correspond to D2-MSN dominant activity. These results indicate that PD mice are dominated by D2-MSN hyperactivity while β-band oscillations are mediated by D1-MSN activity. The β-band oscillations observed in SNr and motor thalamus in PD¹¹ also indicate the direct pathway may still be relevant in PD as we show the indirect pathway β-band oscillations could not propagate to SNr/thalamus while direct pathway could.

We demonstrated a new possibility that the β-band oscillations in healthy and potentially in PD conditions are mediated by both the direct and indirect pathways: the β-band oscillations observed in GPi, SNr and motor thalamus are propagated via D1-MSN; the β-band oscillations observed in GPe and STN are mediated by D2-MSN. We believe that combining optogenetics and computational modeling is a powerful tool in providing cell-type specific, mechanistic explanations to brain functions/dysfunctions and enabling therapeutic optimizations.

### Methods

### Animals

Two bacterial artificial chromosome (BAC)-mediated transgenic mouse lines from the gene expression nervous system atlas (GENSAT)1 were used. Male mice weighing 15-20 g (˜4 weeks old) were used as subjects in the experiments. Mice expressed Cre recombinase under control of either the dopamine D1 receptor (D1-Cre) or dopamine D2 receptor (D2-Cre) regulatory elements. The double-floxed inverted (DIO) recombinant AAV2 virus was used to express ChR2-YFP in Cre-expressing neurons. The recombinant AAV vector was serotyped with AAV1 coat proteins and packaged by the University of North Carolina viral vector core (titer of 4×1012 particles/ml). The viral injection were performed at dorsomedial striatum (+0.4 mm AP, −1.4 mm ML, injection at −2.6 mm DV). To validate functional ChR2 expression in D1- and D2-expressing medium spiny neurons (MSN), and successful control of direct and indirect pathway, behavioral rotation tests were performed during D1- or D2-MSN stimulation. All experimental procedures and animal husbandry were performed in strict accordance with the NIH, UCLA Institutional Animal Care and Use Committee (IACUC), and Stanford University IACUC guidelines.

ofMRI Experiments and Data Analysis

The ofMRI data are adopted from the previous publication of Lee et al 9. Technical details of the ofMRI experiments and ofMRI data analysis are provided in the previous publication, and provided here in brief. fMRI scanning was performed in a 7 Tesla Bruker Bispec small animal MRI system at UCLA. Mice were gradually introduced to fMRI-related phenomena and rewarded with peanut butter over the course of 14 days. A single ofMRI scan consisted of six cycles of 20 s pre-stimulation baseline, 20 s stimulation (20 Hz, 30% duty cycle) and 20 s post-stimulation period. The optical fiber output power was calibrated to 2.5 mW. During fMRI scanning, animals were very lightly anesthetized using isoflurane (0.5-0.7%) mixed with O₂ and N₂O. Brain damage and probe location were examined using T2-weighted high-resolution anatomical images acquired prior to fMRI. Gradient recalled echo (GRE) BOLD methods were used to acquire fMRI images during photostimulation. The fMRI image acquisition was designed to have 25×25 mm² in-plane field of view (FOV) and 0.36×0.36×0.5 mm³ spatial resolution with a sliding window reconstruction to update the image every repetition time (TR). The two-dimensional, multi-slice gradient-echo sequence used a four-interleave spiral readout, 750 ms TR, and 12 ms echo time, resulting in 23 coronal slices. Real-time motion correction was performed using a custom-designed GPU-based system. fMRI data processing was performed using custom-written programs and mrVista (Stanford Vision and Imaging Science and Technology Laboratory, Stanford, CA) in Matlab (MathWorks, Inc., Natick, MA). Motion-corrected images from the scans of the same animal were first averaged together and then aligned to a single, common coordinate frame, using a six-degree-of-freedom rigid body transformation. Time series were calculated for each voxel as the percent modulation of the BOLD signal relative to a 30 s baseline period collected prior to stimulation. The average images from each animal were used to generate mean and standard error estimates of active volume for different ROIs. To visualize the spatial and temporal dynamics of evoked activity, the images from all D1- and D2-Cre animals were averaged together into two groups.

### In Vivo Electrophysiology Recordings of ChR2 Mice

In vivo single unit recordings were conducted at selected sites (CPu, GPi, thalamus and STN) that were modulated by optogenetic stimulations on D1- and D2-MSNs, respectively. Recordings were collected for 20 s without stimulation, followed by repeated stimulation cycles (20 s on, 40 s off) with 20 Hz, 30% duty cycle light delivery. Animals were anesthetized with 0.8-1.0% isoflurane through a nose cone while secured on a stereotactic frame. An acute 16-channel microelectrode array (Neuronexus Technologies) was targeted to the recording site using stereotaxic instruments. Plexon's omniplex system and Plexcontrol software were used to capture and sort the spike waveform data in real-time. Threshold search in Plexon's offline sorter application was used for automated detection of spikes in multi-unit recording, which was then validated by visual inspection and modified if necessary. For traces with multiple spike populations, thresholds were set to capture all the spikes. It is therefore likely that multiple neurons were recorded simultaneously during bursting. Thalamic recordings were targeted to the ventrolateral (VL) motor nucleus. The average recording location across animals are: +0.52 mm AP, 1.64 mm ML, +3.33 mm DV (striatum, n=10 of 11 animals); 1.32 mm AP, 1.08 mm ML, +3.58 mm DV (thalamus, n=9 of 13 animals); 2.06 mm AP, 1.39 mm ML, +4.54 mm DV (STN, n=4 of 4 animals); and 1.34 mm AP, 1.73 mm ML, +4.37 mm DV (GPi, n=5 of 5 animals). Immunohistochemistry was performed to assess the specificity of ChR2-EYFP expression to dopaminergic D1- and D2-MSNs. Although we did not explicitly verify the neuronal types of STN, GPi and thalamus, the recorded neurons showed consistent firing patterns with fMRI. Therefore, we believe most of the neurons we recorded belong to the same class.

### In Vivo Electrophysiology Recordings of SSFO Mice

In vivo electrophysiology was performed to directly measure the neuronal activity of various brain regions during caudate-putamen (CPu) stimulation. As with imaging, anesthesia was maintained with a mixture of O₂ (35%), N₂O (65%), and ˜0.5% isoflurane, Dex 0.025 mg/kg bolus, 0.05 mg/kg-hr infusion. Throughout the procedure, body temperature was maintained at 37° C. using a thermoresistive heating pad (FHC, Inc., ME, USA). After securing the animal within a stereotactic frame, a 16-channel microelectrode array (NeuroNexus Technologies, MI, USA; A1×16 standard model linear electrode array) was inserted at the desired recording site. For recordings at the site of stimulation CPu (+0.86 mm AP, −1.50 mm ML, −3.40 mm DV), an optical fiber glued to the electrode tip was used to deliver light. Remote recordings were performed at the following coordinates, averaged across animals: thalamus; THL (−1.34 mm AP, −1.00 mm ML, −3.40 mm DV), substantia nigra pars reticulata; SNr (−3.25 mm AP, +1.50 mm ML, −4.30 mm DV), globus pallidus interna; GPi (−1.22 mm AP, +2.20 mm ML, −4.10 mm DV), globus pallidus externa; GPe (−1.34 mm AP, −1.90 mm ML, −3.50 mm DV), and. Light was delivered to the fiber-optic implant at CPu via a 473 nm (blue) and 594 nm (yellow) laser source (Laser Glow Technologie) calibrated to 5 mW power delivery for 2 s pulse of 473 nm, 10 s pulse of 594 nm.

For the optrode positioned in CPu, a 200 μm diameter optical fiber was used to deliver continuous light from a 473 nm and 594 nm laser source calibrated to 5 mW at the implanted fiber's tip. Recordings were performed for 538 s without stimulation, followed by a 2 s pulse of 473 nm light for activating SSFO (Stabilized Step Function Opsins, AAV-EF1a-DIO-hChR2 (C128S/D156A)-EYFP), then turned on 594 nm yellow laser for 10 s to de-activate SSFO.

### Dynamical Causal Modeling

Dynamic causal modeling (DCM) is a Bayesian optimization approach that estimates the effective connectivity among brain regions. Spectral DCM is a type of DCM that models the endogenous fluctuations in addition to the causal relations among regions³³. The DCM implemented in current study followed the same procedure in previous publication²⁴ and are described in brief in this section. We improved the segmentation (see FIG. S7 for more details), and the updated DCM results are presented in FIGS. 1 and 2. The classical DCM in the time domain uses a state space system to model the brain dynamics, which is described by:

$$\begin{matrix}\dot{x} ( t ) = f ( x ( t ) , u ( t ) ; {θ}_{f} ) + w ( t ; {θ}_{w} ) \\ y ( t ) = g ( x ( t ) , u ( t ) ; {θ}_{g} ) + ϵ ( t ; {θ}_{ϵ} )\end{matrix}$$

The time series x(t) is the neuronal activity, u(t) is the deterministic input stimuli, which optogenetic stimulation and y(t) is the observed signal, which in our case the ofMRI BOLD signals. w(t) is state noise and ε(t) is observation noise. Classical DCM is the first DCM method developed for task-based fMRI. One major limitation of classical DCM is that intrinsic neuronal fluctuations and noises are not modeled. Although our model covered 7 ipsilateral ROIs directly, we considered the other regions, ipsilateral and contralateral of modeled ROIs, as the sources of noises and fluctuations to our model. Furthermore, basal ganglia and thalamus are known to possess various types of tonically active neurons, and these intrinsic activities are crucial for both classical direct/indirect pathway model and in our own hypothetical mechanisms. Therefore, classical DCM is not suitable for the purpose of the current study.

Spectral DCM is developed on top of classical DCM that predicts cross-spectra from modeling the coupled endogenous fluctuations³³. A main advantage of spectral DCM over classical DCM is that it models intrinsic neuronal fluctuations and noises, that represents the intrinsic fluctuations of the 7 nodes in our model and the noise inputs from other nodes not covered by our model. SPM12 toolbox has two options for the forms of noises in the function spm_csd_fmri_mtf.m. The default neuronal fluctuations and noises in SPM12 toolbox are in 1/f forms. In the main text, we presented spectral DCM results with fluctuations and noises in autoregressive forms, which is in accordance with Bernal-Casas et al., 2017. Based on the electrophysiology recordings we collected for the baseline firings of different ROIs, the intrinsic fluctuations and noises were not in the forms of pink noise (1/f noise) but rather close to white noise (see the power spectrum of experimental CPu baseline firings shown in FIGS. 1 and 2). The results of classical DCM, spectral DCM with 1/f form fluctuations, and spectral DCM with auto-regressive form fluctuations (chosen) are shown in FIG. S3 for comparison.

With optogenetic fMRI (ofMRI) BOLD time series from D1- and D2-MSN β-band (20 Hz) stimulation experiments (FIG. S1a, S1d and Sid), we implemented spectral dynamic causal modeling (DCM) to analyze effective connectivity strengths among regions of interest (ROIs), utilizing the methods from previous publications^{24,33}. 7 ROIs were chosen in the CBT network and the structural connectivity was used as a prior of the spectral DCM analysis (FIG. S1b). The ROIs include the caudate putamen (CPu), external globus pallidus (GPe), internal globus pallidus (GPi), subthalamic nucleus (STN), substantia nigra pars reticulata (SNr), thalamus (THL), and motor cortex (MCX). The 7 ROIs were selected based on recent experimental and modeling literature^{9,34,35}. The thalamus in our model is fitted with data collected from ventral lateral thalamus for its established role motor in control. ofMRI BOLD time series were extracted for the 7 ROIs during D1- and D2-MSN stimulations, shown in FIG. S1D and S1E, respectively. Effective connectivity strengths were estimated for each individual subject (D1: n=12; D2: n=10) with low-pass filtered fMRI time series and then averaged across subjects.

### Large-Scale Biophysical Modeling

Large-scale neural network modeling based on the biophysics attributes of neurons and brain regions accounts for neuronal functions from the single neuron spiking level to neural masses^{36,37}. We constructed 7 modules to represent the 7 ROIs in CBT network. CPu module consisted of 100 Huxley-Hudgkin neurons representing D1-MSNs and 100 representing D2-MSNs. Each of the rest 6 modules consisted of 100 Hodgkin-Huxley neurons. Considering the distinct biophysical properties between the GABAergic projections and the glutamatergic projections, the two types of connections were modeled separately (FIG. 3A).

As shown in FIG. 3, the connections along direct/indirect pathways (GABAergic connections) are optimized using single-neuron recordings alone. As a “closed loop” model, we only used DCM to close the loop by connecting basal ganglia-thalamus pathways to the cortex and forwarding cortical feedbacks to basal ganglia-thalamus (glutamatergic connections). For these connections, we do not investigate further on the synaptic properties but only their effective influences on direct/indirect pathways. The parameters of GABAergic connections are the same for D1- and D2-MSN networks. The glutamatergic connections were connected separately for D1- and D2-MSN stimulations according to DCM results. We only considered the connections estimated by DCM as significant or close-to-significant (p˜[0.05, 0.1]). The glutamatergic connections were modeled as 1-to-1 connections with connection strengths proportional to the effective connectivity estimated by DCM. The proportion ratio was determined as the minimum value that a Hodgkin-Huxley neuron could respond to all DCM significant projections. The GABAergic projections along the direct and indirect pathways are highly diffusive²⁵. We modeled CPu-GPi/GPe and GPi/SNr-thalamus GABAergic projections as 1-to-n diffusive projections with connectivity strength wGABA, where n and wGABA are free parameters. N and wGABA are searched across parameter space until the simulated spike rates matched statistically with the results of extracellular recordings. The model inputs at CPu were determined based on experimental single-neuron recordings (FIG. 3B). For more mathematical details, configuration of DCM, and results on how the DCM results impact biophysical modeling, see FIG. S3. For CPu-GPi, CPu-GPe, and GPi-THL, nis 25 and wGABA is chosen to be −120. For GPe-STN/GPi, n is 75 and wGABA is chosen to be −140. The parameters were chosen according to FIG. 4I as the firing rates with this combination were able to reproduce empirical data from single-neuron recordings and meanwhile biologically realistic. We sampled spike trains from the extracellular recordings of CPu, which were used as inputs of the model (FIG. 3B).

We choose Hodgkin-Huxley neuronal model as the basic unit of our biophysical model³⁸. Since we model experimental data from various scales using fMRI and single-neuron recordings, the biophysical model needs to generate data comparable with our single-neuron recordings. Therefore, we limit our choices of neuronal models to electrical input-output membrane voltage models that can produce simulated spike trains. In this category, we choose the original Hodgkin-Huxley model (implemented with NEST simulator using hh_psc_alph nodes) to best replicate biological activity while limiting the size of parameter space. The integrate-and-fire models are more simplified but neglect many aspects of neuronal features including ion channel petameters.

Hodgkin-Huxley model³⁹ is a conductance-based model that describes the relationship between the ionic currents and the membrane voltage of the neuron. The behavior of the model is controlled by the differential equation:

$${C}_{m} \frac{d V ( t )}{dt} = - {\sum }_{i} {I}_{i} ( t , V ) ,$$

in which Cₘ is the capacitance representing the lipid layer, V is the membrane voltage of the cell, and Iᵢ is the current of ion channel i. The Iᵢ can be described as follows.

$${I}_{i} ( t , V ) = {g}_{i} ( t , V ) ( V - {V}_{m} ) ,$$

where g is the conductance of each channel. The g value for each channel is determined by the maximal conductance g multiplying fractions of activation and inactivation:

$$\begin{matrix}{g}_{K} = {\overline{g}}_{K} {n}^{4} \\ {g}_{N a} = {\overline{g}}_{N a} {m}^{3} h\end{matrix}$$

n, m and h are dimensionless fractions between 0 and 1 representing potassium channel activation, sodium activation and sodium inactivation, respectively.

### Impacts of DCM Results on Biophysical Model's Simulation Outcomes

The same fMRI data and DCM modeling process were utilized in a previous publication on DCM modeling²⁴. To further improve the DCM outcome, we modified the segmentation scheme (see Methodology for details) which mainly affected STN. The fMRI BOLD time series extracted under the new segmentation scheme aligned better with the electrophysiological data, which was not taken into account in Bernal-Casas et al. We replicated connectivity estimates that match the results of Bernal-Casas et al. on polarities and statistical significance level, except for one connection (GPe to SNr). GPe-SNr connection in Bernal-Casas et al. is positive and significant (p<0.05) while in our replication attempt it is positive but non-significant.

In Bernal-Casas et al., all fMRI BOLD time series were detrended with 3^{rd} order nonlinear regression. In our current study, we used the original time series for DCM as we are interested in the excitatory/inhibitory trend caused D1-/D2-MSN stimulation. Detrending the time series affects the DCM amplitudes, but not on the polarities, or statistical significance of connectivity estimates. All fMRI BOLD time series presented in our current study and in Bernal-Casas et al. were demeaned. Demeaning time series does not affect DCM results as the SPM12 toolbox will demean all timeseries automatically before model inversions (version 13 Jan. 2020). The DCM results from our current study and from Bernal-Casas et al. will lead to different simulated electrophysiological data from the biophysical model. The difference occurred mainly in STN spike rates, which is consistent with the segmentation changes on STN. The simulated spike rates of STN matches the experimental data better with the new segmentation (FIG. S3c), which proves the validity of our DCM results.

### Simulated Experiments

We first simulated 20 seconds baseline and then applied 20 Hz stimulation to D1-/D2-MSNs for 20 seconds, which was followed by another 20 seconds simulation without stimulation. We further tested the pervasiveness of D1-MSN stimulations of different frequency bands (FIG. S5e). Along with beta band, low-gamma band (30-60 Hz) stimulations on striatal D1-MSN exhibit highest pervasiveness in basal ganglia, which matches experimental observations 31.

We simulated how neuromodulation on various ROIs could interrupt the beta-oscillations propagated along direct and indirect pathways. The experiment design is shown in FIGS. 6A and 6D. Optogenetic stimulation was implemented as direct activation with different strength (500 pA, 700 pA, and 900 pA), while opto-inhibition was implemented as sustained shunting inhibition on each neuron of the target region⁴⁰. Two types of optogenetic stimulation were implemented: periodic and random stimulation. For periodic stimulation, regular stimulation pulses of 130 Hz were applied to the targeted ROI. Inspired by recent progress on random reset deep-brain stimulation⁴¹, we also simulated random opto-stimulation on various target. 40 stimulation sequences of randomly spaced pulses of 130 Hz were employed. Each neuron of the targeted ROI was stimulated with one of the 40 stimulation sequences. Such design is meant to de-couple the synchronized neurons of the targeted ROI.

### Computing SPIKE-Synchronization

SPIKE-synchronization profiles were calculated with Python package PySpike²³. The values were averaged with 200 ms bins to show the temporal changes of synchronization. The 200 ms bin width was chosen to smooth the curves and meanwhile could still reflect the temporal dynamics. The SPIKE-synchronization is a time-resolved measurement thus the impact of firing rates on the counted synchronization events is already corrected. See Kreuz et al., 2015 for mathematical details.

### Computing Effective Connectivity

The effective connectivity estimates were calculated follow the procedures described in Bernal-Casas et al., 2017. In brief, effective connectivity strengths were estimated for each individual subject (D1: n=12; D2: n=10) with low-pass filtered fMRI time series and then averaged across subjects. The cut-off frequency of low-pass filter was selected in a way that it maximized the number of statistically significant connections and close-to-significant connections (see FIG. S2). Connections, tested against zero using 1-sample t-test, with p values less than 0.05 were considered as significant and p values between 0.05 and 0.1 as close-to-significant.

### Detecting Co-Occurrence of Beta-Epochs

We calculated the temporal profile of the averaged power of beta-band frequencies for each neuron with a time resolution of 350 ms. We then performed pair-wise comparison for the beta-power temporal profiles for all neurons of simultaneously recorded ROIs. Co-occurred peaks of beta-power were counted for each pair. The total amount of co-occurred beta-band oscillation epochs for baseline and stimulation periods were statistically compared using two-sided t-test.

### Statistics

One sample t-tests were used to determine the significances of effective connectivity estimates. For the chosen cut-off frequencies, mean values, p-values and 95% confidence intervals are shown in Tables S1-S6. The number of subjects used for statistical testing across the study were n=12 for D1-MSN stimulation and n=10 for D2-MSN stimulation. One subject from the D2-MSN stimulation group was considered as an outlier and excluded from the statistical testing.

One-tailed paired t-tests were performed in quantifying the differences caused by optogenetic stimulations in the firings rates, SPIKE-synchronization profiles, and corresponding metrics in simulated results shown in FIGS. 1, 2, and 4. Two-tailed paired t-tests were performed in comparing the experimental and simulated firings rates in FIGS. 5 and 6. The number of neurons identified in single-neuron recordings were: in CPu, n=118 for D1-MSN stimulation, n=143 for D2-MSN stimulation; in GPi, n=78 for D1-MSN stimulation, n=266 for D2-MSN stimulation; in thalamus, n=31 for D1-MSN stimulation, n=55 for D2-MSN stimulation; in STN, D1-MSN stimulation n=95; D2-MSN stimulation n=90. We used the Lilliefors test with MATLAB to test the normality of our data. Specifically, we tested for Gaussianity in the connectivity estimates calculated in DCM analysis and the firing rates of neurons identified in single-neuron recordings. In both cases, the null hypothesis that connectivity estimates were normally distributed cannot rejected (p>0.05). Therefore, our choice of classical t-test is justified for testing statistical significances in DCM and firing rates analysis.

### Software

All primary software tools used in this study are available. Dynamic causal modeling was conducted with SPM12 toolbox (fil.ion.ucl.ac.uk/spm). The large-scale biophysical modeling was performed on the NEST simulator (nest-simulator.org).

### REFERENCES

1. Engel, A. K., Fries, P. & Singer, W. Dynamic predictions: Oscillations and synchrony in top-down processing. Nat Rev Neurosci 2, 704-716 (2001).
2. Cannon, J. et al. Neurosystems: brain rhythms and cognitive processing. Eur J Neurosci 39, 705-719 (2014).
3. Schoffelen, J. M., Oostenveld, R. & Fries, P. Neuronal coherence as a mechanism of effective corticospinal interaction. Science 308, 111-113 (2005).
4. Albin, R. L., Young, A. B. & Penney, J. B. The functional anatomy of basal ganglia disorders. Trends Neurosci 12, 366-375 (1989).
5. DeLong, M. R. Primate models of movement disorders of basal ganglia origin. Trends Neurosci 13, 281-285 (1990).
6. Kühn, A. A. et al. Event-related beta desynchronization in human subthalamic nucleus correlates with motor performance. Brain 127, 735-746 (2004).
7. Kravitz, A. V., Tye, L. D. & Kreitzer, A. C. Distinct roles for direct and indirect pathway striatal neurons in reinforcement. Nat Neurosci 15, 816-818 (2012).
8. Goldberg, J. H., Farries, M. A. & Fee, M. S. Basal ganglia output to the thalamus: still a paradox. Trends Neurosci 36, 695-705 (2013).
9. Lee, H. J. et al. Activation of Direct and Indirect Pathway Medium Spiny Neurons Drives Distinct Brain-wide Responses. Neuron 91, 412-424 (2016).
10. Kim, J. et al. Inhibitory Basal Ganglia Inputs Induce Excitatory Motor Signals in the Thalamus. Neuron 95, 1181-1196.e8 (2017).
11. Nakamura, K. C., Sharott, A., Tanaka, T. & Magill, P. J. Input Zone-Selective Dysrhythmia in Motor Thalamus after Dopamine Depletion. J Neurosci 41, 10382-10404 (2021).
12. Fogelson, N. et al. Different functional loops between cerebral cortex and the subthalmic area in Parkinson's disease. Cereb Cortex 16, 64-75 (2006).
13. Heimer, G., Rivlin, M., Israel, Z. & Bergman, H. Synchronizing activity of basal ganglia and pathophysiology of Parkinson's disease. in Parkinson's Disease and Related Disorders (eds. Riederer, P., Reichmann, H., Youdim, M. B. H. & Gerlach, M.) 17-20 (Springer, 2006). doi: 10.1007/978-3-211-45295-0_4.
14. Davis, N. J., Tomlinson, S. P. & Morgan, H. M. The Role of Beta-Frequency Neural Oscillations in Motor Control. J. Neurosci. 32, 403-404 (2012).
15. Feingold, J., Gibson, D. J., DePasquale, B. & Graybiel, A. M. Bursts of beta oscillation differentiate postperformance activity in the striatum and motor cortex of monkeys performing movement tasks. Proc Natl Acad Sci USA 112, 13687-13692 (2015).
16. Leventhal, D. K. et al. Basal Ganglia Beta Oscillations Accompany Cue Utilization. Neuron 73, 523-536 (2012).
17. Schmidt, R., Leventhal, D. K., Mallet, N., Chen, F. & Berke, J. D. Canceling actions involves a race between basal ganglia pathways. Nat Neurosci 16, 1118-1124 (2013).
18. Brown, P. et al. Effects of stimulation of the subthalamic area on oscillatory pallidal activity in Parkinson's disease. Experimental Neurology 188, 480-490 (2004).
19. Eusebio, A. et al. Deep brain stimulation can suppress pathological synchronisation in parkinsonian patients. J Neurol Neurosurg Psychiatry 82, 569-573 (2011).
20. Silberstein, P. et al. Cortico-cortical coupling in Parkinson's disease and its modulation by therapy. Brain 128, 1277-1291 (2005).
21. Mallet, N. et al. Parkinsonian Beta Oscillations in the External Globus Pallidus and Their Relationship with Subthalamic Nucleus Activity. J. Neurosci. 28, 14245-14258 (2008).
22. McCarthy, M. M. et al. Striatal origin of the pathologic beta oscillations in Parkinson's disease. PNAS 108, 11620-11625 (2011).
23. Kreuz, T., Bozanic, N. & Mulansky, M. SPIKE-Synchronization: a parameter-free and time-resolved coincidence detector with an intuitive multivariate extension. BMC Neurosci 16, P170 (2015).
24. Bernal-Casas, D., Lee, H. J., Weitz, A. J. & Lee, J. H. Studying Brain Circuit Function with Dynamic Causal Modeling for Optogenetic fMRI. Neuron 93, 522-532.e5 (2017).
25. Parent, A. et al. Organization of the basal ganglia: the importance of axonal collateralization. Trends in Neurosciences 23, S20-S27 (2000).
26. Crompe, B. de la et al. The globus pallidus orchestrates abnormal network dynamics in a model of Parkinsonism. Nat Commun 11, 1570 (2020).
27. Mallet, N. et al. Disrupted Dopamine Transmission and the Emergence of Exaggerated Beta Oscillations in Subthalamic Nucleus and Cerebral Cortex. J. Neurosci. 28, 4795-4806 (2008).
28. Yttri, E. A. & Dudman, J. T. Opponent and bidirectional control of movement velocity in the basal ganglia. Nature 533, 402-406 (2016).
29. Parker, J. G. et al. Diametric neural ensemble dynamics in parkinsonian and dyskinetic states. Nature 557, 177-182 (2018).
30. Schwerdt, H. N. et al. Dopamine and beta-band oscillations differentially link to striatal value and motor control. Science Advances 6, eabb9226 (2020).
31. Kondabolu, K. et al. Striatal cholinergic interneurons generate beta and gamma oscillations in the corticostriatal circuit and produce motor deficits. PNAS 113, E3159-E3168 (2016).
32. Zemel, D. et al. Dopamine depletion selectively disrupts interactions between striatal neuron subtypes and LFP oscillations. Cell Reports 38, 110265 (2022).
33. Friston, K. J., Kahan, J., Biswal, B. & Razi, A. A DCM for resting state fMRI. Neuroimage 94, 396-407 (2014).
34. Calabresi, P., Picconi, B., Tozzi, A., Ghiglieri, V. & Di Filippo, M. Direct and indirect pathways of basal ganglia: a critical reappraisal. Nat Neurosci 17, 1022-1030 (2014).
35. Schroll, H. & Hamker, F. H. Computational models of basal-ganglia pathway functions: focus on functional neuroanatomy. Front. Syst. Neurosci. 7, (2013).
36. Deco, G., Jirsa, V. K., Robinson, P. A., Breakspear, M. & Friston, K. The Dynamic Brain: From Spiking Neurons to Neural Masses and Cortical Fields. PLoS Computational Biology 4, e1000092 (2008).
37. Liu, Q., Ulloa, A. & Horwitz, B. Using a Large-scale Neural Model of Cortical Object Processing to Investigate the Neural Substrate for Managing Multiple Items in Short-term Memory. Journal of Cognitive Neuroscience 29, 1860-1876 (2017).
38. Jordan, J. et al. NEST 2.18.0. (2019) doi: 10.5281/zenodo.2605422.
39. Hodgkin, A. L. & Huxley, A. F. A quantitative description of membrane current and its application to conduction and excitation in nerve. The Journal of Physiology 117, 500-544 (1952).
40. Wiegert, J. S., Mahn, M., Prigge, M., Printz, Y. & Yizhar, O. Silencing Neurons: Tools, Applications, and Experimental Constraints. Neuron 95, 504-529 (2017).
41. Khaledi-Nasab, A., Kromer, J. A. & Tass, P. A. Long-Lasting Desynchronization of Plastic Neural Networks by Random Reset Stimulation. Frontiers in Physiology 11, (2021).

### Example 2

Solving Brain Circuit Function and Dysfunction with Computational Modeling and Optogenetic fMRI

Can we systematically design treatments for brain disorders such as Parkinson's Disease? To enable this, we need a full algorithmic description, beyond simple correlations, of how specific cells or brain regions cooperatively contribute to the behavior or symptom in the context of whole-brain network. What does it take to generate such algorithms? Cell types cannot be ignored because even neurons that are in the same location of the brain can drive completely opposite functions and resulting behaviors (1, 2). Neurons often interact with large networks across the whole brain. A limited field of view within the brain is thus insufficient to understand these algorithms. Therefore, in order to obtain the data necessary to reconstruct these algorithms of behavioral control, we need an imaging system that can measure cell-type-specific, whole-brain function. Optogenetic functional magnetic resonance imaging (ofMRI) (3) has begun to achieve this goal. With ofMRI, we can select cell-type-specific modulation targets while monitoring the outcome of such modulation across the whole brain, in vivo with high spatiotemporal resolution. This has opened a new window into the study of brain function. We can see how modulating specific elements of the brain leads to specific behaviors of interest, while also directly observing the inner workings of the brain that led to such behaviors. Through computational modeling of ofMRI-signal measured across the whole-brain (4), cell-type-specific, large-scale brain function can be quantitatively described at the regional level. Once regional interaction maps are reconstructed, we envision that biophysical modeling can be combined to enable cell-type-specific, large-scale modeling of brain function at the single-cell-spiking level. In addition, while restoring brain function is the ultimate goal of neurological disease treatment, understanding how prominent pathology relates to brain function is also of critical importance. In this review, we will discuss approaches taken to date, and approaches that can be employed in the future towards these goals.

Bridging Scales with Optogenetic fMRI.

ofMRI is a technology that combines optogenetic stimulation with fMRI readout. Optogenetics (5, 6) enables cell-type-specific, millisecond-scale, activity modulation using light while high-field fMRI measures the resulting hemodynamic responses in live subjects across the whole brain. In the initial proof-of-concept study (3), motor cortex excitatory neurons triggered fMRI responses that could be measured throughout the brain with sub-second temporal resolution. To accelerate scientific discovery with ofMRI, several technological innovations were made. Real-time imaging with robustness to the live subject's motion that achieves data acquisition, reconstruction, motion correction (7), and analysis of 3D images with high accuracy in approximately 12 ms was developed. To resolve cortical layer and sub-nuclei specific responses, novel compressed sensing (8-10) and machine learning based fMRI technology was developed, which achieved significant reduction in voxel volume. MR-compatible optrodes and electrodes were also developed for simultaneous electrophysiological recordings to validate the neural basis of the ofMRI hemodynamic signal (11, 12). They can achieve simultaneous acquisition of electrophysiology recordings during fMRI sessions, and provide information with higher temporal resolution in regions of interest identified by ofMRI.

Utilizing these advanced ofMRI technologies, capabilities and precision of ofMRI has been extensively tested. It has been shown that location, polarity, and temporal shape of neural activity can be accurately inferred from the ofMRI signal (3, 13, 14), and that neural activity can be measured by ofMRI across multiple synapses (11, 15). Stimulation cell-type, location, frequency (14, 15), and intensity (11) was shown to dramatically change the location and shape of activities throughout the brain. It was also made clear that whole-brain neural dynamics as measured by ofMRI can accurately predict distinct behaviors (2, 14, 15).

Many studies have used ofMRI to improve our understanding of fundamental circuitries associated with behavior, memory, and cognition. For instance, ofMRI studies identified that frequency-dependent thalamic activities drive distinct whole-brain function in circuits associated with arousal, attention, and somatosensory function (16-18). ofMRI studies revealed distinct dorsal and ventral hippocampal control of brain-wide function (15, 19), circuits associated with cell-type-specific targeting of somatosensory cortex (20), and cerebellar cortex functional control over forebrain and midbrain (21). Another study revealed brain-wide dynamics that govern how the medial prefrontal cortex regulates reward-related behaviors through distant regions such as the striatum (22). A recent study has tried fMRI with cell-type-specific activation of astrocytes (23).

These studies have shown that the observed hemodynamic activities are closely tied to neuronal activities using either simultaneous or follow-up electrophysiology. Furthermore, although most ofMRI studies have been conducted in rodents, it has also been applied to non-human primates, where both saccade latencies and whole-brain activity were found to be dependent on specific neuronal targets in the motor cortex (24).

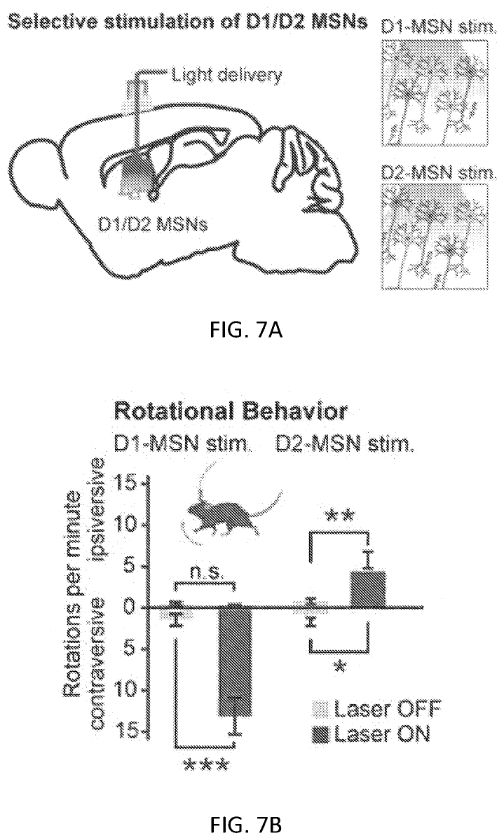

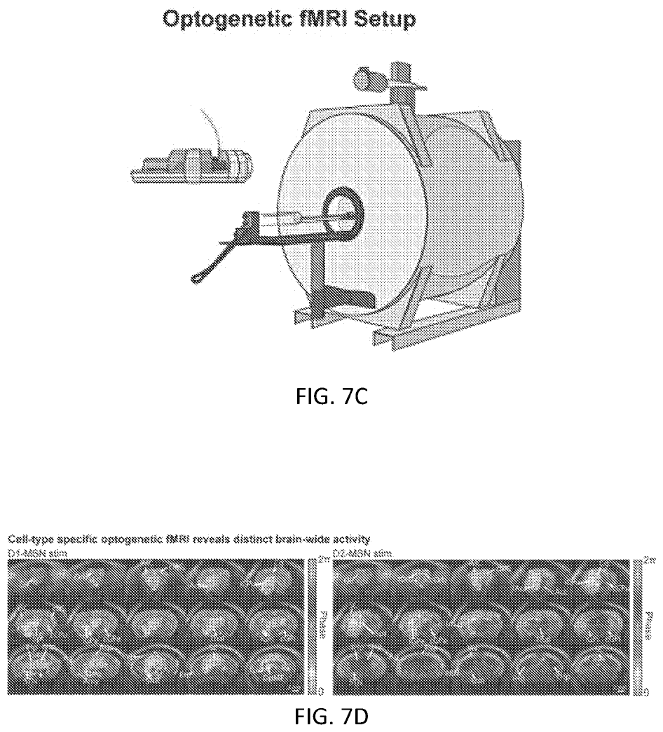

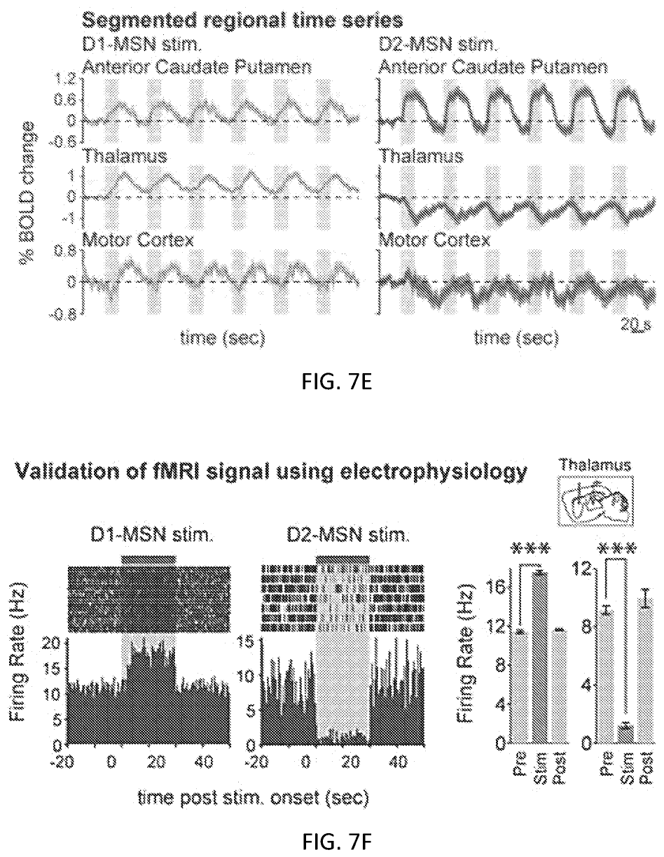

The ability to probe and readout whole-brain activity with ofMRI has also advanced our understanding of dysfunctional circuitry associated with neurological disorders. For studying epilepsy, ofMRI provided a unique advantage of being able to optogenetically-induce seizures with precise origins on demand, while measuring the resulting whole-brain activities with simultaneous electrophysiology recordings. This enabled studies that can generate models to predict and classify seizures using its early activity markers (15, 25). Furthermore, longitudinal effects of seizures on global brain function could be measured to understand how the disease progresses and how seizures are generated and maintained (26). These advances aid our understanding of circuit mechanisms underlying seizures, help design intervention parameters, such as stimulation location and frequency to effectively inhibit seizures. Optogenetic fMRI can also elucidate mechanisms behind existing therapies, such as poststroke recovery. Activation as measured by ofMRI was highly predictive of the degree of recovery, and identified sensory circuits involved in this process (27). We can now start to reveal detailed circuit mechanisms that were challenging to understand before. As an example, we will review studies uncovering how D1- and D2-receptor expressing medium spiny neurons (MSNs) dynamically regulate global brain function and dysfunction (FIG. 7). The cortico-basal-ganglia-thalamus circuit is implicated in many important brain functions including motor control and reward mechanisms. Neurological disorders such as Parkinson's Disease, Huntington's Disease, addiction, and autism involve this network. The caudate putamen seats the D1- and D2-MSNs (FIG. 7A), which is the key node that separates the direct and indirect pathways (FIG. 7B). To assess the brain-wide dynamics driven by inhibitory D1- and D2-MSNs, Lee et al. performed whole-brain fMRI during repeated 20 s periods of optogenetic D1- or D2-MSN stimulations (2) (FIG. 7C). Active voxels were identified as those significantly synchronized to the repeated stimulations (FIG. 7D). The local signal at the site of stimulation was positive for both inhibitory D1- and D2-MSN stimulations (FIG. 7E), shedding light on a widely debated issue whether activity of inhibitory neurons evokes a positive or negative fMRI signal. At most regions of the ipsilateral cortico-basal-ganglia-thalamus network, the evoked response in a given region exhibited qualitatively different temporal profiles between D1- and D2-MSN stimulations (FIGS. 7D and 7E). Given the diversity of BOLD responses evoked by D1- and D2-MSN stimulations, we sought to verify whether the responses reflected underlying neuronal activity using single-unit recordings. For example, the fMRI time series in thalamus exhibited robust and reliable increases and decreases upon D1- and D2-MSN stimulations, respectively (FIGS. 7D and 7E). Indeed, single-units exhibited an increase in firing rate during D1-MSN stimulations and a decrease in firing rate during D2-MSN stimulations (FIG. 7F). These results demonstrate that ofMRI can detect cell-type-specific multi-synaptic activity changes across the whole brain with high spatiotemporal precision, and that electrophysiological recordings alongside ofMRI can support these findings.

As with any technology, ofMRI has caveats for future improvements as well as potential pitfalls that need to be avoided. Channelrhodopsin (ChR2) is known to evoke synchronized neuronal activity upon light stimulation. Therefore, before launching an ofMRI investigation, it is important to first investigate the behavioral impact of the optogenetic stimulations, as a mean to ensure that the behavior generated is of interest in either normal physiological or pathological context. For example, in our D1- and D2-MSN stimulation ofMRI experiments, increased contralateral and ipsilateral rotations were observed, respectively (2) (FIG. 7B). This shows that the two separate stimulations result in opposite behaviors known to be associated with movement disorders. The fact that ChR2 evokes synchronization upon light stimulation also makes it suitable for studying pathological oscillations in a number of disease models/contexts. Excessive beta-band oscillations in Parkinson's Disease have been extensively explored with ChR2 induced oscillations (28, 29). Some newer opsins, such as stabilized step function opsin (SSFO) (30), modulate target neurons by increasing the excitability to amplify existing spontaneous activity, which expands the application range of ofMRI to more physiological conditions. There are many aspects of ofMRI that can be further improved. For example, ofMRI will benefit greatly from higher spatio-temporal resolution, real-time feedback-based stimulation control, and imaging during behaviors (7, 31).

It is also important to note other technologies that have been developed for the investigation of brain circuitries, including high-speed volumetric calcium imaging (32, 33), probes for high-density electrophysiology recordings (34, 35), and widefield calcium and voltage imaging (36). Compared to ofMRI, these new advances offer higher spatiotemporal resolution, although limited by recording depths and field of view. For example, widefield “whole-cortex” calcium imaging enables up to 30 Hz simultaneous recording of cortical regions, but its depth coverage is limited to superficial layers of cortex. Combining fMRI and widefield calcium imaging has been shown to mitigate the limitations of both methods (36). Therefore, future ofMRI studies could be integrated and with other technologies for complementary strengths.

### Cell-Type-Specific Modeling of Large-Scale Brain Function.

Large-scale neural network models (37-41) utilizing experimental data from PET (42), fMRI (43), EEG/MEG (44) have made significant contributions to understanding brain functions. However, although these modeling efforts are based on data from carefully designed experiments that attempt to isolate specific brain function, multiple networks and pathways mediated by different cell types are simultaneously involved in orchestrating a brain function. Therefore, without the capability to untangle contributions from different cell types across the whole brain, models have been limited in their capabilities. The development of ofMRI technology opens a new opportunity in terms of whole-brain computational modeling because it uniquely measures cell-type-specific whole-brain dynamics.

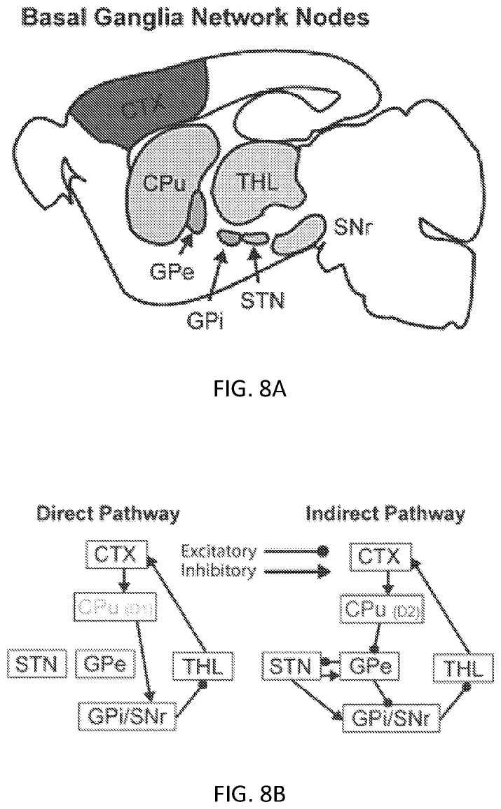

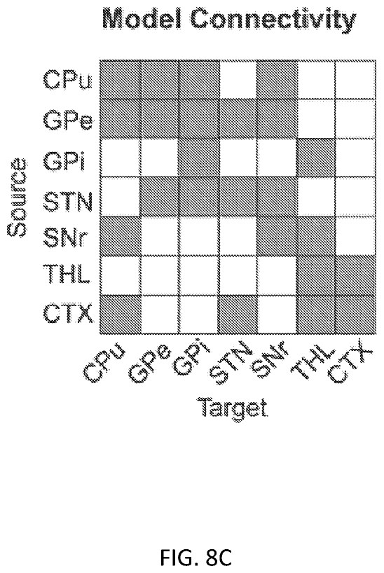

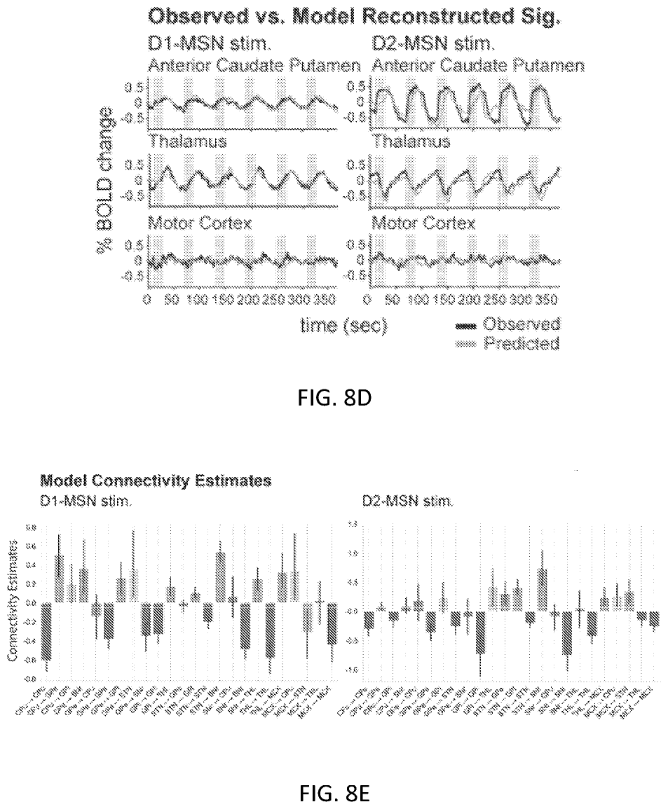

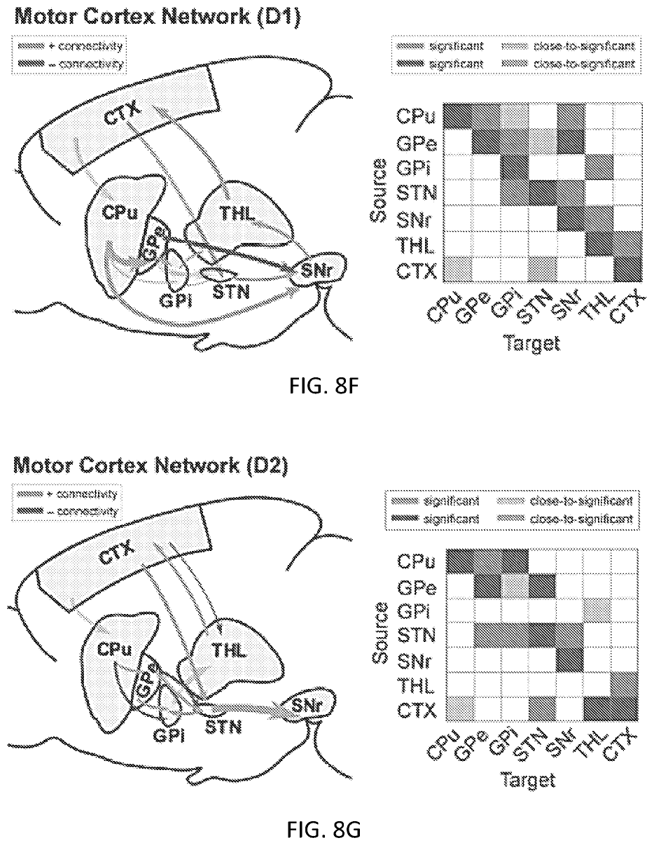

For example, the cortico-basal-ganglia-thalamus network features a large number of network nodes (FIG. 8A) and distinct cell types that have distinct long-range projections. According to the canonical functional model of basal ganglia (45, 46), movement initiation and suppression are mediated by the direct and indirect pathways, respectively (FIG. 8B). The direct pathway promotes movements through D1-MSN excitation in the striatum followed by excitation in the thalamus and consequently the motor cortex. On the other hand, the indirect pathway is assumed to transfer the activation of D2-MSN in the striatum via complex interactions within the basal ganglia, resulting in inhibition of the thalamus and consequently the motor cortex. However, despite the wide usage and success of the canonical model in explaining experimental results, recent experimental evidence argues against the model and finds the interactions between the two pathways still puzzling. For example, the direct and indirect pathways operate simultaneously during movement onsets (47).

As demonstrated earlier with ofMRI, we can decompose the operation of the cortico-basal-ganglia-thalamus network by optogenetically activating D1- and D2-MSNs selectively and directly observing the corresponding brain-wide dynamics (2) (FIG. 7). However, the degree of connectivity in the cortico-basal-ganglia-thalamus network is too high (FIGS. 8B and 8C) to identify regional causal influences based on the time series alone (FIG. 8D). Through computational modeling such as dynamic causal modeling (DCM), with the precision afforded by the ofMRI data, cell-type-specific causal linkages among regions of interest (ROIs) in the cortico-basal-ganglia-thalamus network could be identified.

DCM is a modeling scheme that estimates the causal coupling (effective connectivity) in a multi-region network based on neuroimaging data (fMRI, MEG/EEG) (48-50). The estimations are fitted to empirical results using Bayesian techniques. One major strength of DCM is that the estimated regional connectivities are directional, which is especially valuable for networks with a lot of reciprocal connections and feedbacks like the cortico-basal-ganglia-thalamus network. ofMRI can also be combined with other modeling schemes. For example, Salvan et al. (51) optogenetically modulated the entorhinal cortex and combined hidden Markov modeling with ofMRI data to study how entorhinal cortex drives frequency-dependent brain-wide dynamic states. In another study, multivariate dynamical systems (MDS) causal modeling was used with ofMRI to estimate causal brain interactions (52). Like DCM, MDS models both intrinsic and experimentally induced causal couplings in a large-scale brain network.

In our previous study (4), spectral DCM (38), a variation of DCM that enables large-scale networks to be modeled with computational efficiency, was used to investigate the interactions within the cortico-basal-ganglia-thalamus network with ofMRI data from D1- and D2-MSN optogenetic stimulations. One recent study reported consistent results using DCM with D1- and D2-MSN stimulation ofMRI data (53). As illustrated in FIG. 8, combining DCM or equivalent modeling schemes with ofMRI data can accurately reveal brain-wide regional interactions. The time series is also accurately reproduced by DCM, closely matching experimental ofMRI time series (FIG. 8D). FIGS. 8E-8G show the DCM estimations of between-region effective connectivity (4) utilizing ofMRI data. DCM results verified the direct pathway activation during D1-MSN stimulation and indirect pathway activation during D2-MSN stimulation. The defining connections of direct pathway model are statistically significant (CPu to SNr, GPi to thalamus, SNr to thalamus) or close-to-significant (CPu to GPi) with D1-MSN stimulations. In the D2-MSN stimulation network, significant connections included those of indirect pathway (CPu to GPe, GPe to STN, STN to GPi/SNr, and GPi to thalamus). The existence of cortical feedbacks can also be observed (FIGS. 8F and 8G). On the other hand, the effective connectivity estimates by DCM also suggest several positive projections that are anatomically inhibitory and cannot be explained by the canonical direct/indirect pathway model, such as the projections from GPi to thalamus during D1-MSN stimulations, which matches several experimental reports of GPi-thalamus paradoxical coactivation (2, 54-56). Understanding the mechanism underlying such paradoxical connections requires further microscopic investigations into the specific synaptic interactions with techniques like single-cell-spiking level modeling and single-unit recordings, as we will discuss next.

### Cell-Type-Specific, Single-Cell-Spiking Level Modeling of Brain-Wide Function.

One key strength of computational modeling is that it can bridge between spatial scales, from whole-brain dynamics to single-cell activity, and explain data from different experimental modalities, from fMRI to extracellular recordings (57, 58). DCM and other macroscale and mesoscale brain models commonly use neural mass models or mean-field models as the basic unit which describes the collective neural activity in a brain region or a cortical column (42-44). On the other hand, single-cell-spiking models computationally depict the microscopic biophysical features of how single-cell level spiking controls and modulates brain functions/dysfunctions (59-61).

Despite having the ability to capture and model the macroscale and mesoscale interactions between separate regional populations of neurons in the brain with ofMRI and DCM, there is certainly an added advantage in modeling the interactions between individual neurons of varying cell-type. Neurological disorders may differentially impact specific cell populations within one brain region. Optogenetically stimulating one type of cell population, while inhibiting another type in external globus pallidus (GPe) prolonged the therapeutic effects on a mouse model of Parkinson's Disease (52). It is widely assumed and supported by optogenetics studies that Parkinson's Disease causes hyperactivity of D2-MSNs and hypoactivity of D1-MSNs, thus impairing the balance between the direct and indirect pathways (46, 47, 62). DYT1 dystonia, a genetic early onset dystonia, is related to cholinergic interneuron dysfunction and altered D2 receptor-function in striatum (63, 64). Firing pattern alterations of one cell type may also contribute to large-scale changes. Optogenetic stimulation in striatal cholinergic interneurons, a subpopulation constituting less than 2% of the striatum, could generate broad-band oscillations in the motor network (29). With cell-type-specific, single-cell-spiking level modeling, it is easier to address the heterogeneity and rich microscopic interactions within one region with biophysical details. ofMRI-based DCM or other regional brain dynamics model can serve as a bridge between whole-brain dynamics and single-cell-spiking level activity, enabling construction of large-scale, cell-type-specific biophysical models that can test neuronal-level hypotheses.

Cell-type-specific, single-cell-spiking level models that can accurately predict circuit function and dysfunction can be very powerful tools for designing or optimizing therapy. Thus far, many large-scale models without cell type specificity have been constructed to test various hypotheses underlying deep-brain stimulation (DBS), an existing therapy for Parkinson's Disease (65-67).

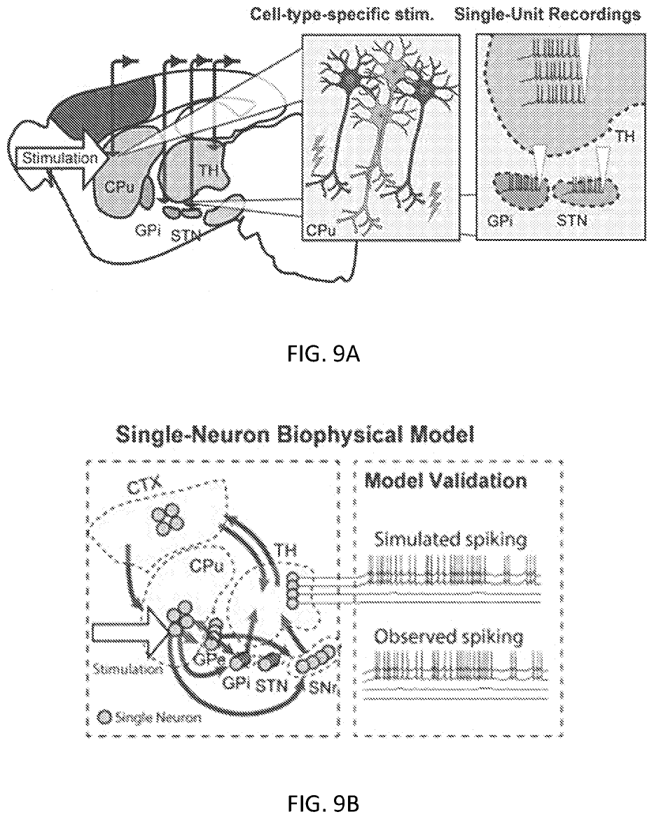

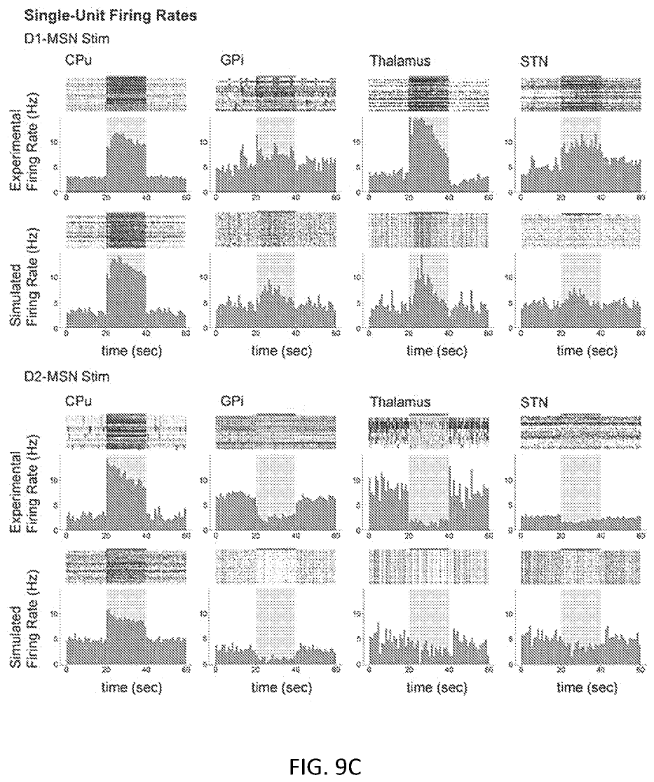

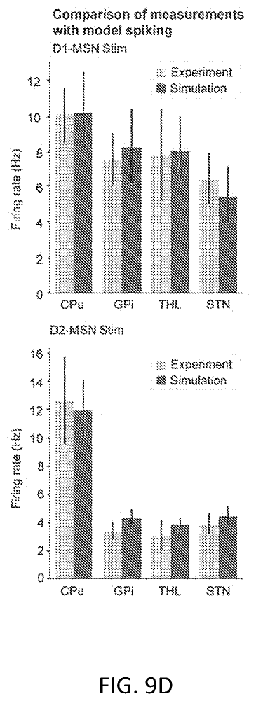

However, because each cell type in the basal ganglia has distinct synaptic and physiological properties, accurate models would need cell-type-specific parameters that can fit cell-type-specifically acquired experimental data. We envision that ofMRI, DCM, and electrophysiological recordings can be combined to build large-scale biophysical models that can model brain-wide activity at the single-cell-spiking level. FIG. 9A demonstrates this vision. With ofMRI and electrophysiology experiments providing multi-scale data, and DCM providing brain-wide interaction information, single-cell level biophysical models, with accurate cell-type-specific, large-scale context, can be built and validated (FIG. 9B). As illustrated in FIGS. 9C and 9D, a single-cell-spiking level model can be designed to reproduce experimental neuronal activity and group dynamics with high precision. With such models, we can imagine simulating brain-wide spiking activity from “virtual neuromodulations” without needing to do in vivo experiments. We would then be able to use these results to better optimize therapeutic targets and parameters where simulated spiking matches our desired response. For example, a recent study (68) employed optogenetics and single-cell level computational modeling within three different cell types of the motor cortex to show that DBS on cortical somatostatin interneurons can rescue Parkinsonian symptoms. We envision that in the future, ofMRI-enabled brain-wide models with single-cell-spiking level precision will ultimately enable systematic design of neuromodulation therapy with predictable outcome.

### Modeling Whole-Brain Pathology Dynamics and its Relationship to Brain Function.

The brain circuitry is relevant to neurological disorders beyond its utility in modeling local and global brain function. It is also important for understanding the underlying pathology of many disorders. In the case of Parkinson's Disease and other synucleinopathies, the seeding and gradual accumulation of pathological alpha-synuclein (α-syn) causes dopaminergic neurodegeneration, which ultimately leads to striatal imbalance and the cardinal Parkinsonian signs (69). Although the specific biochemical trafficking mechanisms of these proteins remain unknown, several recent studies have shown that whole-brain spreading patterns are highly dependent on the inoculation site (70), and that anatomical connectivity is highly predictive of susceptible regions after induced seeding of α-syn pathology (71).

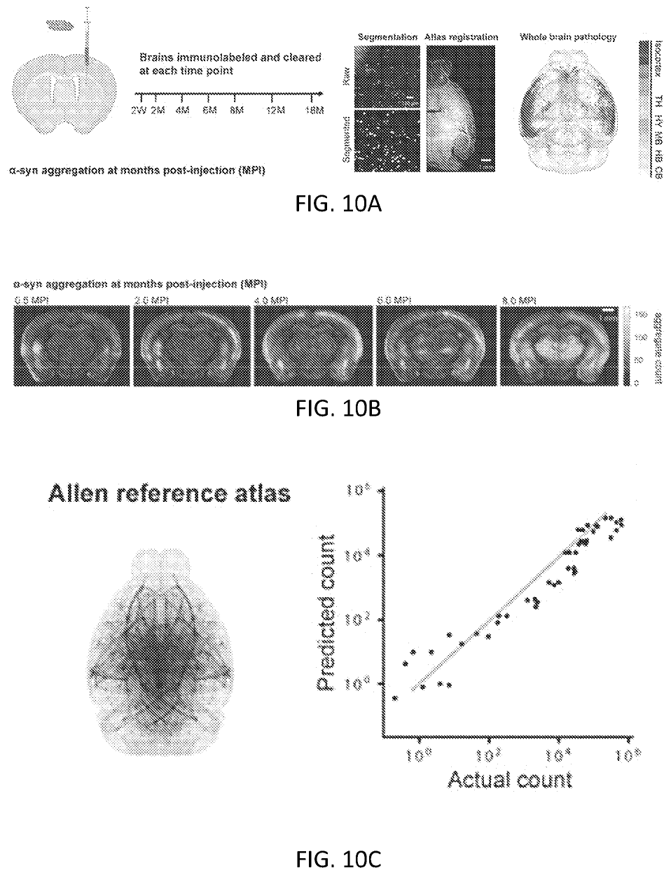

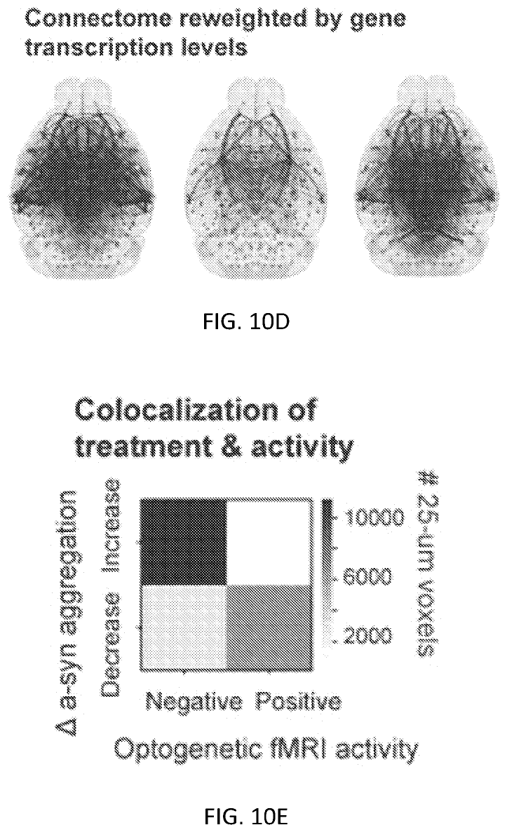

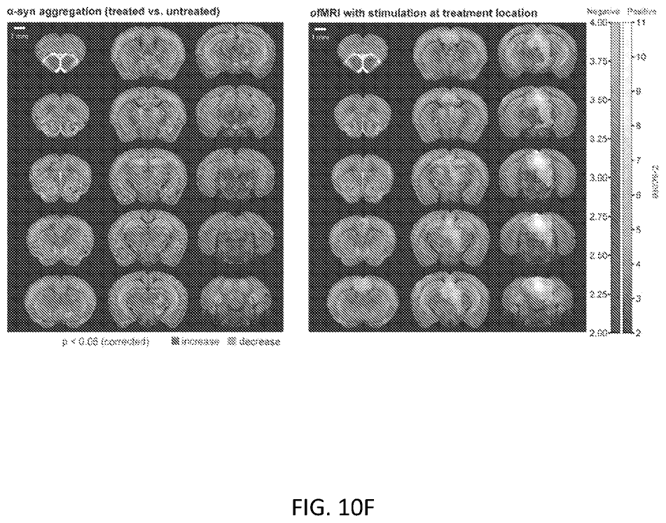

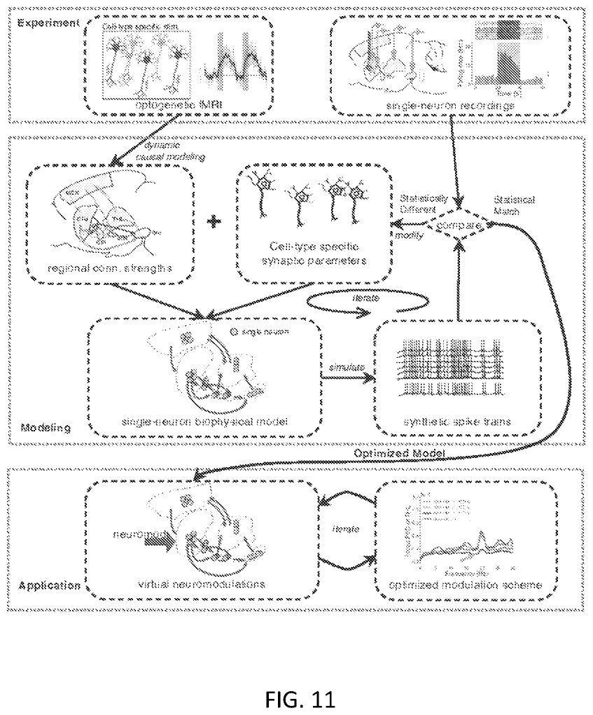

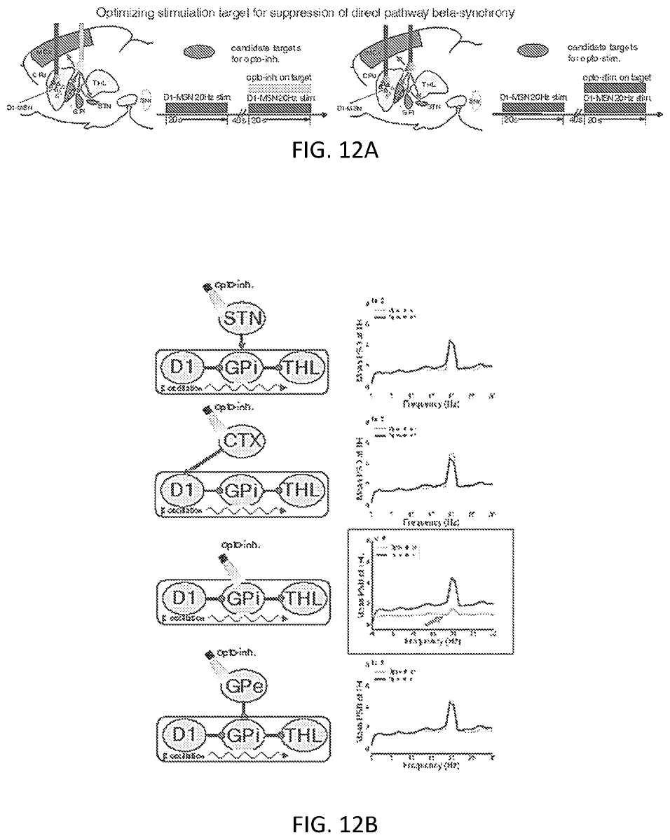

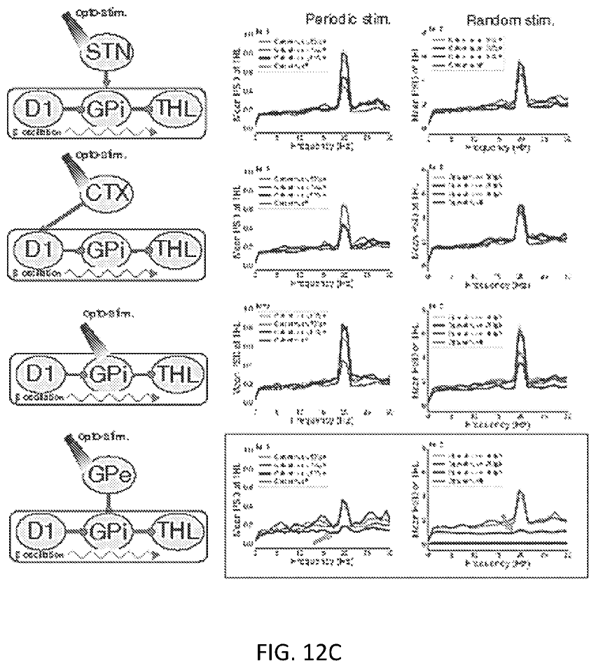

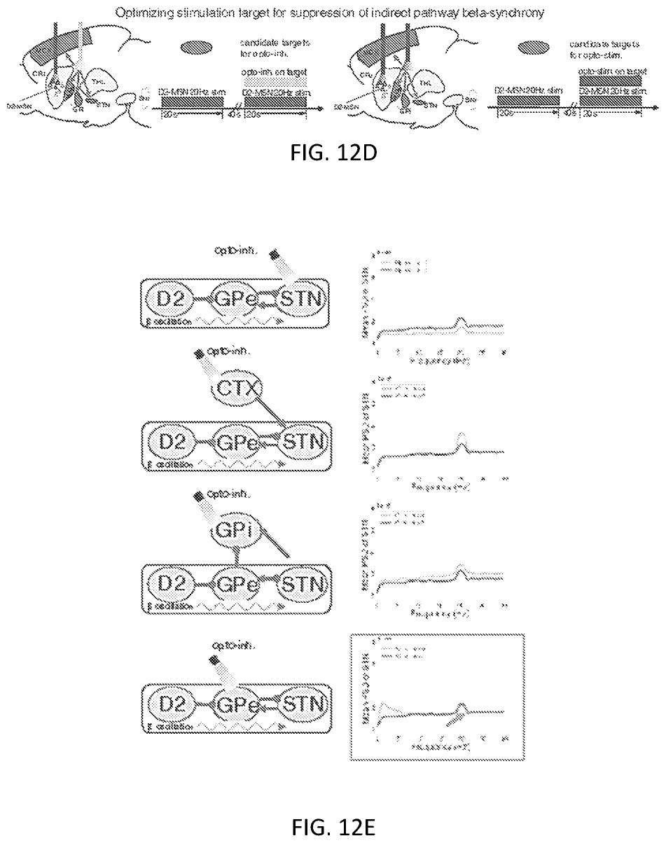

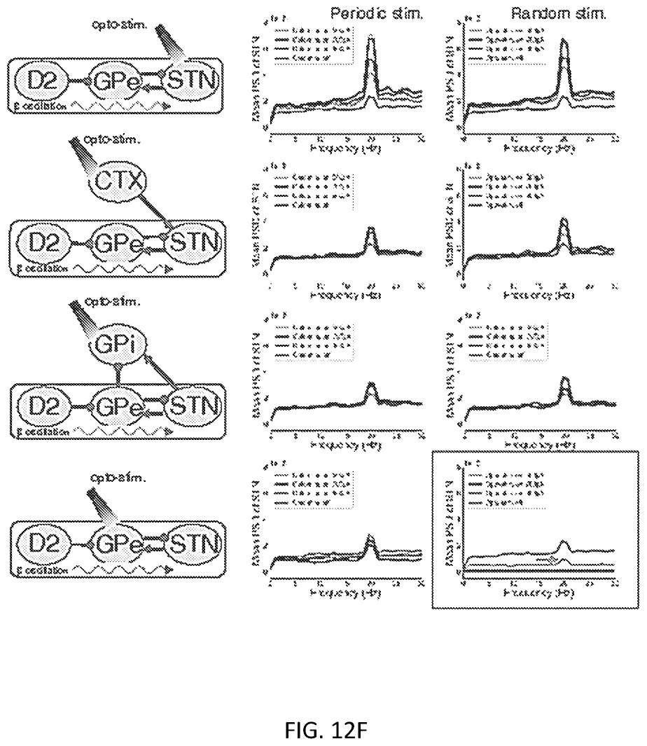

To understand the linkage between anatomical connections, pathology, and function, it is important to reliably measure the whole-brain pathological state. The last decade has seen several prominent advancements in whole-brain tissue clearing technology. These range from hydrogel-based techniques such as CLARITY (72) that enable multiplexed brain imaging but potentially require long incubation times, to solvent-based techniques like 3DISCO (73) that are generally faster but may bleach fluorescent signal. Combining these tissue clearing methods with whole-brain immunolabeling and light-sheet microscopy allows for high-resolution examination of whole-brain pathology using biochemical reporters (74, 75). As these three-dimensional whole-brain histological datasets now provide micron resolution and are exceeding terabytes in size, it is necessary to have automated registration and segmentation techniques (76) to capture the rich information provided by the data. FIG. 10A depicts how a Parkinsonian disease model where the injection of α-synuclein PFFs are used to trigger whole-brain pathology can be systematically analyzed using computational pipeline. Brains can be immunolabeled and cleared at each time point using the iDISCO method (74), with the imaged aggregates automatically segmented and registered to a standardized atlas. As depicted in FIG. 10B, longitudinal analysis of pathology can demonstrate a highly dynamic, brain-wide process of spreading after seeding of α-syn, consistent with numerous histological studies (69, 71). Registration to a standardized atlas can provide an enormous advantage enabling systematic statistical comparisons and analysis of disease pathology alongside connectomic (77), genetic (78), and vascular (79) databases, which all reside in the same reference coordinate space. Most notably, the Allen Connectivity Atlas (77) has been crucial in the development of models describing the spread of pathology through the connectome (71). For instance, network diffusion models represent the whole-brain circuitry as a directed graph with neuroanatomical regions as nodes and axonal pathways as edges between these nodes (FIG. 10C). They have found wide applicability in predicting the spread of α-syn pathology in both rodent and human imaging studies (71, 80), as well as in human dementia (81) and supranuclear palsy neuroimaging studies (82). In FIG. 10C, we present an example of how a whole-brain model can accurately reconstruct the regional variability in pathology. Compared to previous models that depended on serial histological sectioning (71) or in vivo human imaging data (80), this type of modeling can provide a more comprehensive, higher resolution description of pathology dynamics that includes all brain regions, which is important for quantitative model descriptions. Given that transgenic mice such as those with modified LRRK2 (71, 83) or SNCA (69) expression demonstrate significantly altered spreading patterns, models can additionally incorporate the brain's inherent genetic or cell-type-specific differences. As depicted in FIG. 10D, re-weighting the connectivity matrix by regional expression of a gene-of-interest can allow for the evaluation of that gene's relevance in disease spread. Henderson et al. weighted a network diffusion model with SNCA expression, demonstrating that in silico simulation of circuits and genes can recover observed α-synuclein pathology (71).

Accurate whole-brain models of pathology dynamics that can predict both future and past states of these progressive disorders will have significant clinical implications. For example, a computational model that takes an arbitrary pathological state and predicts future states could guide interventions that depend on the predicted localization of pathology. Similarly, a model that can iterate backwards and compute previous states, even back to the initial seeding site, can further aid in disease progression classification for diagnosis purposes. Since neurodegenerative disorders like Parkinson's Disease, Lewy Body Dementia, and Multiple System Atrophy have long been hypothesized to collectively form a neuropathological spectrum (84), modeling the various circuits that drive different pathologies and clinical manifestations will play a large part in precisely defining these disorders.

While functional and histological readouts provide distinct information regarding the brain and disease, their colocalization is likely to be important in both furthering our understanding of neural circuitry and developing novel therapeutics. For instance, combining ofMRI and whole-brain pathological labeling could aid in the development of therapeutics that treat the underlying pathology, such as α-syn pathology in Parkinson's Disease. We give an example of spatial colocalization between optogenetic stimulation induced changes, and ofMRI-measured functional changes observed during the delivery of the same stimulation (FIGS. 10E and 10F). This optogenetic stimulation paradigm reduces the α-synuclein aggregate count at the site of stimulation and in various downstream regions. When colocalized with brain activity, their similar spatial locations but opposing polarities are apparent, revealing that positive activity is colocalized with decreases in pathology, while negative activity is highly colocalized with increases in pathology.

This ability to target pathology with neuromodulation, while predicting subsequent brain-wide changes in pathology could provide a new way of thinking about neuromodulation therapy for Parkinson's Disease and related disorders. For instance, parameters such as duration and site of neuromodulation could be tailored to patients based on their current pathological state and neuromodulation parameter's expected impact on pathology. Taking it one step further, therapy could even be designed while taking the expected future states of pathology into consideration.

Altogether, the ability to readout and model pathology with brain clearing while measuring whole-brain network function with optogenetic fMRI will allow for the development of circuit-based models that bridge pathology and function.

### Conclusion

These recent advancements in cell-type-specific neuromodulation, whole-brain functional imaging, and computational modeling are starting to pave a path for a significant turning point in neuroscience. We aim to establish new approaches to simulating brain function that can replicate and predict behaviors of interest. This will transform treatments of a wide array of neurological diseases including Parkinson's Disease.

### REFERENCES AND NOTES

1. P. Calabresi, B. Picconi, A. Tozzi, V. Ghiglieri, M. Di Filippo, Direct and indirect pathways of basal ganglia: a critical reappraisal. Nat Neurosci 17, 1022-1030 (2014).
2. H. J. Lee et al., Activation of Direct and Indirect Pathway Medium Spiny Neurons Drives Distinct Brain-wide Responses. Neuron 91, 412-424 (2016).
3. J. H. Lee et al., Global and local fMRI signals driven by neurons defined optogenetically by type and wiring. Nature 465, 788-792 (2010).
4. D. Bernal-Casas, H. J. Lee, A. J. Weitz, J. H. Lee, Studying Brain Circuit Function with Dynamic Causal Modeling for Optogenetic fMRI. Neuron 93, 522-532 e525 (2017).
5. F. Zhang, A. M. Aravanis, A. Adamantidis, L. de Lecea, K. Deisseroth, Circuit-breakers: optical technologies for probing neural signals and systems. Nat Rev Neurosci 8, 577-581 (2007).
6. A. M. Aravanis et al., An optical neural interface: in vivo control of rodent motor cortex with integrated fiberoptic and optogenetic technology. J Neural Eng 4, S143-156 (2007).
7. Z. Fang, J. H. Lee, High-throughput optogenetic functional magnetic resonance imaging with parallel computations. J Neurosci Methods 218, 184-195 (2013).
8. Z. Fang, N. Le, M. Choy, J. H. Lee, High spatial resolution compressed sensing (HSPARSE) functional magnetic resonance imaging. Magn Reson Med In press, (2015).
9. D. J. Holland et al., Compressed Sensing Reconstruction Improves Sensitivity of Variable Density Spiral fMRI. Magnetic Resonance in Medicine 70, 1634-1643 (2013).
10. X. P. Zong, J. Y. Lee, A. J. Poplawsky, S. G. Kim, J. C. Ye, Compressed sensing fMRI using gradient-recalled echo and EPI sequences. Neuroimage 92, 312-321 (2014).
11. B. A. Duffy, M. Choy, M. R. Chuapoco, M. Madsen, J. H. Lee, MRI compatible optrodes for simultaneous LFP and optogenetic fMRI investigation of seizure-like afterdischarges. Neuroimage, (2015).
12. M. R. Chuapoco et al., Carbon monofilament electrodes for unit recording and functional MRI in same subjects. Neuroimage 186, 806-816 (2019).
13. B. Byers et al., Direct in vivo assessment of human stem cell graft-host neural circuits. Neuroimage 114, 328-337 (2015).
14. J. Liu et al., Frequency-selective control of cortical and subcortical networks by central thalamus. Elife 4, (2015).
15. A. J. Weitz et al., Optogenetic fMRI reveals distinct, frequency-dependent networks recruited by dorsal and intermediate hippocampus stimulations. Neuroimage 107, 229-241 (2015).
16. J. Liu et al., Frequency-selective control of cortical and subcortical networks by central thalamus. Elife 4, e09215 (2015).
17. A. J. Weitz, H. J. Lee, M. Choy, J. H. Lee, Thalamic Input to Orbitofrontal Cortex Drives Brain-wide, Frequency-Dependent Inhibition Mediated by GABA and Zona Incerta. Neuron 104, 1153-1167 e1154 (2019).
18. A. T. Leong et al., Long-range projections coordinate distributed brain-wide neural activity with a specific spatiotemporal profile. Proc Natl Acad Sci USA 113, E8306-E8315 (2016).
19. N. Takata et al., Optogenetic activation of CA1 pyramidal neurons at the dorsal and ventral hippocampus evokes distinct brain-wide responses revealed by mouse fMRI. PLoS One 10, e0121417 (2015).
20. W. B. Jung, H. Jiang, S. Lee, S. G. Kim, Dissection of brain-wide resting-state and functional somatosensory circuits by fMRI with optogenetic silencing. Proc Natl Acad Sci USA 119, (2022).
21. K. Y. Choe, C. F. Sanchez, N. G. Harris, T. S. Otis, P. J. Mathews, Optogenetic fMRI and electrophysiological identification of region-specific connectivity between the cerebellar cortex and forebrain. Neuroimage 173, 370-383 (2018).
22. E. A. Ferenczi et al., Prefrontal cortical regulation of brainwide circuit dynamics and reward-related behavior. Science 351, aac9698 (2016).
23. N. Takata et al., Optogenetic astrocyte activation evokes BOLD fMRI response with oxygen consumption without neuronal activity modulation. Glia 66, 2013-2023 (2018).
24. A. Gerits et al., Optogenetically induced behavioral and functional network changes in primates. Curr Biol 22, 1722-1726 (2012).
25. B. A. Duffy, M. Choy, J. H. Lee, Predicting Successful Generation and Inhibition of Seizure-like Afterdischarges and Mapping Their Seizure Networks Using fMRI. Cell Rep 30, 2540-2554 e2544 (2020).
26. M. Choy et al., Repeated hippocampal seizures lead to brain-wide reorganization of circuits and seizure propagation pathways. Neuron 110, 221-236 e224 (2022).
45 27. S. Vahdat et al., Brain-wide neural dynamics of poststroke recovery induced by optogenetic stimulation. Sci Adv 7, (2021).
28. B. Crompe et al., The globus pallidus orchestrates abnormal network dynamics in a model of Parkinsonism. Nat Commun 11, 1570 (2020).
29. K. Kondabolu et al., Striatal cholinergic interneurons generate beta and gamma oscillations in the corticostriatal circuit and produce motor deficits. Proc Natl Acad Sci USA 113, E3159-3168 (2016).
30. O. Yizhar et al., Neocortical excitation/inhibition balance in information processing and social dysfunction. Nature 477, 171-178 (2011).
31. Z. N. Fang, N. V. Le, M. Choy, J. H. Lee, High Spatial Resolution Compressed Sensing (HSPARSE) Functional MRI. Magnetic Resonance in Medicine 76, 440-455 (2016).
32. J. Demas et al., High-speed, cortex-wide volumetric recording of neuroactivity at cellular resolution using light beads microscopy. Nature Methods 18, 1103-+ (2021).
33. V. Voleti et al., Real-time volumetric microscopy of in vivo dynamics and large-scale samples with SCAPE 2.0. Nature Methods 16, 1054-+ (2019).
34. N. A. Steinmetz et al., Neuropixels 2.0: A miniaturized high-density probe for stable, long-term brain recordings. Science 372, (2021).
35. R. Hultman et al., Brain-wide Electrical Spatiotemporal Dynamics Encode Depression Vulnerability. Cell 173, 166-180 e114 (2018).
36. E. M. R. Lake et al., Simultaneous cortex-wide fluorescence Ca (2+) imaging and whole-brain fMRI. Nat Methods 17, 1262-1271 (2020).
37. O. Sporns, Networks of the brain. (The MIT Press, Cambridge, Massachusetts; London, England, ed. Paperback edition. 2016).
38. A. R. McIntosh, Towards a network theory of cognition. Neural Netw 13, 861-870 (2000).
39. X. J. Wang, Neurophysiological and Computational Principles of Cortical Rhythms in Cognition. Physiological Reviews 90, 1195-1268 (2010).
40. G. Deco, M. L. Kringelbach, Great Expectations: Using Whole-Brain Computational Connectomics for Understanding Neuropsychiatric Disorders. Neuron 84, 892-905 (2014).
41. A. Fornito, A. Zalesky, M. Breakspear, The connectomics of brain disorders. Nat Rev Neurosci 16, 159-172 (2015).
42. M. A. Tagamets, B. Horwitz, Integrating electrophysiological and anatomical experimental data to create a large-scale model that simulates a delayed match-to-sample human brain imaging study. Cereb Cortex 8, 310-320 (1998).
43. K. E. Stephan et al., Nonlinear dynamic causal models for fMRI. Neuroimage 42, 649-662 (2008).
44. S. J. Kiebel, O. David, K. J. Friston, Dynamic causal modelling of evoked responses in EEG/MEG with lead field parameterization. Neuroimage 30, 1273-1284 (2006).
45. R. L. Albin, A. B. Young, J. B. Penney, The functional anatomy of basal ganglia disorders. Trends Neurosci 12, 366-375 (1989).
46. M. R. DeLong, Primate models of movement disorders of basal ganglia origin. Trends Neurosci 13, 281-285 (1990).
47. J. G. Parker et al., Diametric neural ensemble dynamics in parkinsonian and dyskinetic states. Nature 557, 177-182 (2018).
48. K. J. Friston, L. Harrison, W. Penny, Dynamic causal modelling. Neuroimage 19, 1273-1302 (2003).
49. K. J. Friston, J. Kahan, B. Biswal, A. Razi, A DCM for resting state fMRI. Neuroimage 94, 396-407 (2014).
50. K. E. Stephan et al., Ten simple rules for dynamic causal modeling. Neuroimage 49, 3099-3109 (2010).
51. P. Salvan et al., Frequency modulation of entorhinal cortex neuronal activity drives distinct frequency-dependent states of brain-wide dynamics. Cell Rep 37, 109954 (2021).
52. S. Ryali et al., Combining optogenetic stimulation and fMRI to validate a multivariate dynamical systems model for estimating causal brain interactions. Neuroimage 132, 398-405 (2016).
53. C. Grimm et al., Optogenetic activation of striatal D1R and D2R cells differentially engages downstream connected areas beyond the basal ganglia. Cell Rep 37, 110161 (2021).
54. J. Kim et al., Inhibitory Basal Ganglia Inputs Induce Excitatory Motor Signals in the Thalamus. Neuron 95, 1181-1196 e1188 (2017).
55. B. C. Schwab et al., Neural activity during a simple reaching task in macaques is counter to gating and rebound in basal ganglia-thalamic communication. PLoS Biol 18, e3000829 (2020).
56. J. H. Goldberg, M. A. Farries, M. S. Fee, Basal ganglia output to the thalamus: still a paradox. Trends Neurosci 36, 695-705 (2013).
57. G. Deco, V. K. Jirsa, P. A. Robinson, M. Breakspear, K. Friston, The dynamic brain: from spiking neurons to neural masses and cortical fields. PLoS Comput Biol 4, e1000092 (2008).
58. M. Schirner, A. R. McIntosh, V. Jirsa, G. Deco, P. Ritter, Inferring multi-scale neural mechanisms with brain network modelling. Elife 7, (2018).
59. J. J. J. Hjorth et al., The microcircuits of striatum in silico. Proc Natl Acad Sci USA 117, 9554-9565 (2020).
60. X. Zhang, S. Santaniello, Role of cerebellar GABAergic dysfunctions in the origins of essential tremor. Proc Natl Acad Sci USA 116, 13592-13601 (2019).
61. M. J. Bezaire, I. Raikov, K. Burk, D. Vyas, I. Soltesz, Interneuronal mechanisms of hippocampal theta oscillations in a full-scale model of the rodent CA1 circuit. Elife 5, (2016).
62. A. V. Kravitz et al., Regulation of parkinsonian motor behaviours by optogenetic control of basal ganglia circuitry. Nature 466, 622-626 (2010).
63. K. L. Eskow Jaunarajs, P. Bonsi, M. F. Chesselet, D. G. Standaert, A. Pisani, Striatal cholinergic dysfunction as a unifying theme in the pathophysiology of dystonia. Prog Neurobiol 127-128, 91-107 (2015).
64. G. Sciamanna et al., Optogenetic Activation of Striatopallidal Neurons Reveals Altered HCN Gating in DYT1 Dystonia. Cell Rep 31, 107644 (2020).
65. J. M. Meier et al., Virtual deep brain stimulation: Multiscale co-simulation of a spiking basal ganglia model and a whole-brain mean-field model with the virtual brain. Exp Neurol, 114111 (2022).
66. W. J. Neumann et al., Functional segregation of basal ganglia pathways in Parkinson's disease. Brain 141, 2655-2669 (2018).
67. E. M. Adam, E. N. Brown, N. Kopell, M. M. Mccarthy, Deep brain stimulation in the subthalamic nucleus for Parkinson's disease can restore dynamics of striatal networks. Proc Natl Acad Sci USA 119, e2120808119 (2022).
68. S. Valverde et al., Deep brain stimulation-guided optogenetic rescue of parkinsonian symptoms. Nat Commun 11, 2388 (2020).
69. K. C. Luk et al., Pathological alpha-synuclein transmission initiates Parkinson-like neurodegeneration in nontransgenic mice. Science 338, 949-953 (2012).
70. Y. J. Guo, H. Xiong, K. Chen, J. J. Zou, P. Lei, Brain regions susceptible to alpha-synuclein spreading. Mol Psychiatry 27, 758-770 (2022).
71. M. X. Henderson et al., Spread of alpha-synuclein pathology through the brain connectome is modulated by selective vulnerability and predicted by network analysis. Nat Neurosci
22, 1248-1257 (2019). 72. K. Chung, K. Deisseroth, CLARITY for mapping the nervous system. Nat Methods 10, 508-513 (2013).
73. A. Erturk et al., Three-dimensional imaging of solvent-cleared organs using 3DISCO. Nat Protoc 7, 1983-1995 (2012).
74. N. Renier et al., iDISCO: a simple, rapid method to immunolabel large tissue samples for volume imaging. Cell 159, 896-910 (2014).
75. T. Liebmann et al., Three-Dimensional Study of Alzheimer's Disease Hallmarks Using the iDISCO Clearing Method. Cell Reports 16, 1138-1152 (2016).
76. N. Renier et al., Mapping of Brain Activity by Automated Volume Analysis of Immediate Early Genes. Cell 165, 1789-1802 (2016).
77. S. W. Oh et al., A mesoscale connectome of the mouse brain. Nature 508, 207-214 (2014).
78. E. S. Lein et al., Genome-wide atlas of gene expression in the adult mouse brain. Nature 445, 168-176 (2007).
79. C. Kirst et al., Mapping the Fine-Scale Organization and Plasticity of the Brain Vasculature. Cell 180, 780-795 e725 (2020).
80. S. Pandya et al., Predictive model of spread of Parkinson's pathology using network diffusion. Neuroimage 192, 178-194 (2019).
81. A. Raj, A. Kuceyeski, M. Weiner, A network diffusion model of disease progression in dementia. Neuron 73, 1204-1215 (2012).
82. S. Pandya, C. Mezias, A. Raj, Predictive Model of Spread of Progressive Supranuclear Palsy Using Directional Network Diffusion. Frontiers in Neurology 8, (2017).
83. G. Bieri et al., LRRK2 modifies alpha-syn pathology and spread in mouse models and human neurons. Acta Neuropathol 137, 961-980 (2019).
84. J. W. Langston, The Parkinson's complex: Parkinsonism is just the tip of the iceberg. Annals of Neurology 59, 591-596 (2006).

What is claimed is:

1. A method of suppressing β-band oscillations in the brain of a subject by performing optogenetic neuromodulation of medium spiny neurons according to a method comprising:
(a) optogenetically inhibiting D1-medium spiny neurons (D1-MSNs) in a globus pallidus internal (GPi) region of the brain of the subject, wherein D1-MSN mediated β-band oscillations are suppressed;
(b) optogenetically inhibiting D2-medium spiny neurons (D2-MSNs) in a globus pallidus external (Gpe) region of the brain of the subject, wherein D2-MSN mediated β-band oscillations are suppressed;
(c) optogenetically stimulating the D1-MSNs periodically in the Gpe region of the brain of the subject, wherein D1-MSN mediated β-band oscillations are suppressed; or
(d) optogenetically stimulating the D1-MSNs and the D2-MSNs randomly in the Gpe region of the brain of the subject, wherein D1-MSN mediated β-band oscillations and D2-MSN mediated β-band oscillations are suppressed; or
any combination of (a)-(d).

2. The method of claim 1, wherein the method comprises:
(a) optogenetically inhibiting the D1-MSNs in the GPi region of the brain of the subject, wherein the D1-MSN mediated β-band oscillations are suppressed;
(b) optogenetically inhibiting the D2-MSNs in the Gpe region of the brain of the subject, wherein the D2-MSN mediated β-band oscillations are suppressed;
(c) optogenetically stimulating the D1-MSNs periodically in the Gpe region of the brain of the subject, wherein the D1-MSN mediated β-band oscillations are suppressed; and
(d) optogenetically stimulating the D1-MSNs and the D2-MSNs randomly in the Gpe region of the brain of the subject, wherein the D1-MSN mediated β-band oscillations and the D2-MSN mediated β-band oscillations are suppressed.

3. The method of claim 1 or 2, wherein said optogenetically inhibiting the D1-MSNs or the D2-MSNs in the Gpi region or the Gpe region comprises:
introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and
illuminating the light-responsive ion channel with light at a wavelength that activates the light-responsive ion channel, wherein conduction of ions by the light-responsive ion channel in response to absorption of light results in hyperpolarization and inhibition of the D1-MSNs or the D2-MSNs.

4. The method of claim 3, wherein the light-responsive ion channel is a light-responsive anion-conducting opsin or a light-responsive proton conductance regulator.

5. The method of claim 4, wherein the light-responsive anion-conducting opsin conducts chloride ions (Cl⁻).

6. The method of claim 4 or 5, wherein the anion-conducting opsin is an anion-conducting channelrhodopsin or halorhodopsin.

7. The method of claim 6, wherein the halorhodopsin is a Natronomonas pharaonis halorhodopsin (NpHR), enhanced NpHR (eNpHR) 1.0, eNpHR 2.0, or eNpHR 3.0.

8. The method of claim 6, wherein the anion-conducting channelrhodopsin is iC1C2, SwiChR, SwiChR++, or iC++.

9. The method of claim 4, wherein the light-responsive proton conductance regulator is a bacteriorhodopsin or an archaerhodopsin.

10. The method of claim 9, wherein the light-responsive proton conductance regulator is Arch from Halorubrum sodomense, ArchT from Halorubrum sp., TP009 from Leptosphaeria maculans, or Mac from Leptosphaeria maculans.

11. The method of claim 1 or 2, wherein said optogenetically stimulating the D1-MSNs or the D2-MSNs in the Gpi region or the Gpe region comprises:
introducing a recombinant polynucleotide encoding a light-responsive ion channel into the D1-MSNs or the D2-MSNs in the Gpi region or the Gpe region, wherein the light-responsive ion channel is expressed in the D1-MSNs or the D2-MSNs; and
illuminating the light-responsive ion channel with light at a wavelength that activates the light-responsive ion channel, wherein conduction of ions by the light-responsive ion channel in response to absorption of light results in depolarization and activation of the D1-MSNs or the D2-MSNs.

12. The method of claim 11, wherein the light-responsive ion channel is a light-responsive cation-conducting opsin.

13. The method of claim 12, wherein the light-responsive cation-conducting opsin conducts calcium cations (Ca²⁺).

14. The method of claim 12 or 13, wherein the light-responsive cation-conducting opsin is a light-responsive cation-conducting channelrhodopsin.

15. The method of claim 14, wherein the light-responsive cation-conducting channelrhodopsin is a Chlamydomonas reinhardtii channelrhodopsin or a Volvox carteri channelrhodopsin.

16. The method of claim 15, wherein the light-responsive cation-conducting channelrhodopsin is a Chlamydomonas reinhardtii channelrhodopsin-1 (ChR1), a Chlamydomonas reinhardtii channelrhodopsin-2 (ChR2), a Volvox carteri channelrhodopsin-1 (VChR1), or a chimeric ChR1-VChR1 channelrhodopsin.

17. The method of any one of claims 1-16, wherein the polynucleotide encoding the light-responsive ion channel is provided by a viral vector.

18. The method of claim 17, wherein the viral vector is a lentiviral vector or an adeno-associated viral (AAV) vector.

19. The method of claim 17 or 18, wherein the viral vector is stereotactically injected into the retrosplenial cortex.

20. The method of any one of claims 17-19, wherein the vector further comprises a neuron-specific promoter operably linked to the polynucleotide encoding the light-responsive ion channel.

21. The method of any one of claims 17-20, wherein expression of the light-responsive ion channel is inducible.

22. The method of any one of claims 1-21, wherein said illuminating the light-responsive ion channel comprises delivering light from a light source to the light-responsive ion channel using a fiber-optic-based optical neural interface.

23. The method of claim 22, wherein the light source is a solid-state diode laser.

24. The method of any one of claims 1-23, wherein the subject has Parkinson's disease.

25. The method of any one of claims 1-24, wherein said optogenetically inhibiting the D1-MSNs comprises sustained shunting inhibition of the D1-MSNs in the GPi region of the brain of the subject.

26. The method of any one of claims 1-25, wherein said optogenetically inhibiting the D2-MSNs comprises sustained shunting inhibition of the D2-MSNs in the GPe region of the brain of the subject.

27. The method of any one of claims 1-26, wherein said optogenetically stimulating the D1-MSNs periodically in the Gpe region of the brain of the subject comprises performing periodic stimulation at a frequency of 130 Hz.

28. The method of any one of claims 1-27, wherein said optogenetically stimulating the D1-MSNs and the D2-MSNs randomly in the Gpe region of the brain of the subject comprises using a plurality of stimulation sequences with randomly spaced pulses of 130 Hz, wherein each neuron is stimulated with one of the stimulation sequences such that synchronized neurons are decoupled.

29. The method of any one of claims 1-28, wherein said optogenetically stimulating the D1-MSNs or the D2-MSNs comprises direct activation of the D1-MSNs or the D2-MSNs with a strength of 500 pA, 700 pA, or 900 pA.

30. A method of treating Parkinson's disease in a subject, the method comprising:
positioning a first electrode at a first location in a globus pallidus internal (GPi) region of the brain of the subject to deliver electrical stimulation to D1-medium spiny neurons in the Gpi region;
positioning a second electrode at a second location in a globus pallidus external (Gpe) region of the brain of the subject to deliver electrical stimulation to D1-medium spiny neurons and D2-medium spiny neurons in the Gpe region; and
applying electrical stimulation to the Gpi region of the brain of the subject using the first electrode and applying electrical stimulation to the Gpe region of the brain of the subject using the second electrode in a manner effective to suppress β-band oscillations to treat Parkinson's disease.

31. The method of claim 30, wherein the electrical stimulation is applied with the first electrode or the second electrode unilaterally or bilaterally.

32. The method of claim 30 or 31, wherein the first electrode or the second electrode is a depth electrode or a surface electrode.

33. The method of any one of claims 30-32, wherein the first electrode or the second electrode is a non-brain penetrating surface electrode array or a brain-penetrating electrode array.

34. The method of any one of claims 30-33, wherein the first electrode is placed on a surface of the Gpi region.

35. The method of any one of claims 30-34, wherein the second electrode is placed on a surface of the Gpe region.

36. The method of any one of claims 30-35, wherein the method further comprises assessing effectiveness of the treatment in the subject using a visual analog scale, a verbal rating scale, a Movement Disorder Society-Sponsored Revision of the Unified Parkinson's Disease Rating Scale (MDS-UPDRS), a Hoehn and Yahr (HnY) scale, or a Montreal Cognitive Assessment (MoCA) scale.

37. A computer implemented method for modeling propagation of β-band oscillations in a brain of a subject and response to neuromodulation, the computer performing steps comprising:
a) receiving functional magnetic resonance imaging data of neural activity before optogenetic stimulation and during optogenetic stimulation of D1-medium spiny neurons (D1-MSNs) and D2-medium spiny neurons (D2-MSNs) in a caudate putamen (CPu) region, an external globus pallidus (GPe) region, an internal globus pallidus (GPi) region, a subthalamic nucleus (STN) region, a substantia nigra pars reticulata (SNr) region, a thalamus (THL) region, and a motor cortex (MCX) region of the brain of the subject;
b) performing spectral dynamic causal modeling of effective connectivity strengths among the CPu region, the GPe region, the GPi region, the STN region, the SNr region, the THL region, and the MCX region;
c) receiving experimental electrophysiological data for the CPu region;
d) estimating effective connectivity strengths of GABAergic connections using the experimental electrophysiological data for the CPu region;
e) estimating effective connectivity strengths of glutamatergic connections using dynamic causal modeling of the functional magnetic resonance imaging data;
f) performing biophysics modeling using a Hodgkin-Huxley model to generate simulated electrophysiology data using the effective connectivity strength estimates;
g) optimizing GABAergic projections iteratively until the simulated electrophysiology data matches the experimental electrophysiological data for the CPu region;
h) calculating a temporal profile of average power of beta-band frequencies for each neuron in the CPu region, the GPe region, the GPi region, the STN region, the SNr region, the THL region, and the MCX region; and
i) comparing total amount of co-occurred beta-band oscillation power before optogenetic stimulation to co-occurred beta-band oscillation power during optogenetic stimulation to model the propagation of β-band oscillations in the brain of the subject.

38. The computer implemented method of claim 37, wherein the D1-MSN are Huxley-Hudgkin neurons.

39. The computer implemented method of claim 37 or 38, wherein the experimental electrophysiology data comprise single-neuron recordings.

40. The computer implemented method of claim 39, wherein the single-neuron recordings are from GABAergic neurons of the CPu region.

41. The computer implemented method of any one of claims 37-40, wherein glutamatergic connections are modeled as 1-to-1 connections with connection strengths proportional to effective connectivity estimated by the DCM.

42. The computer implemented method of any one of claims 39-41, wherein CPu-GPi/GPe and GPi/SNr-thalamus GABAergic projections are modeled as 1-to-n diffusive projections with connectivity strength wGABA, where n and wGABA are free parameters, wherein N and wGABA are searched across parameter space until the simulated spike rates match statistically with the single-neuron recordings.

43. The computer implemented method of any one of claims 37-42, wherein the optogenetic stimulation comprises optogenetically inhibiting the D1-MSNs in the GPi region of the brain of the subject.

44. The computer implemented method of claim 43, wherein said optogenetically inhibiting the D1-MSNs comprises sustained shunting inhibition of the D1-MSNs in the GPi region of the brain of the subject.

45. The computer implemented method of any one of claims 37-44, wherein the optogenetic stimulation comprises optogenetically inhibiting the D2-MSNs in the GPe region of the brain of the subject.

46. The computer implemented method of claim 45, wherein said optogenetically inhibiting the D2-MSNs comprises sustained shunting inhibition of the D2-MSNs in the GPe region of the brain of the subject.

47. The method of any one of claims 37-46, wherein the optogenetic stimulation comprises direct activation of the D1-MSNs or the D2-MSNs with a strength of 500 pA, 700 pA, or 900 pA.

48. The computer implemented method of any one of claims 37-47, wherein the optogenetic stimulation comprises optogenetically stimulating the D1-MSNs periodically in the Gpe region of the brain of the subject.

49. The computer implemented method of any one of claims 37-48, wherein the optogenetic stimulation comprises optogenetically stimulating the D1-MSNs and the D2-MSNs randomly in the Gpe region of the brain of the subject using a plurality of stimulation sequences with randomly spaced pulses, wherein each neuron is stimulated with one of the stimulation sequences such that synchronized neurons are decoupled.

50. The computer implemented method of any one of claims 37-49, wherein the optogenetic stimulation is performed with periodic stimulation at a frequency of 130 Hz.

51. A system for modeling propagation of β-band oscillations in a brain of a subject using the computer implemented method of any one of claims 37-50, the system comprising:
a) a storage component for storing data, wherein the storage component has instructions for modeling propagation of β-band oscillations based on analysis of the functional magnetic resonance imaging data and the experimental electrophysiology data stored therein;
b) a computer processor for processing the functional magnetic resonance imaging data and the experimental electrophysiology data using one or more algorithms, wherein the computer processor is coupled to the storage component and configured to execute the instructions stored in the storage component in order to receive the inputted functional magnetic resonance imaging data and the experimental electrophysiology data and analyze the data according to the computer implemented method of any one of claims 37-50; and
c) a display component for displaying the information regarding the propagation of β-band oscillations in the brain of the subject.

52. A non-transitory computer-readable medium comprising program instructions that, when executed by a processor in a computer, causes the processor to perform the method of any one of claims 37-50.

53. A kit comprising the non-transitory computer-readable medium of claim 52 and instructions for modeling the propagation of β-band oscillations from the functional magnetic resonance imaging data and the experimental electrophysiology data.
